(19) United States
(12) Patent Application Publication
(10) Pub. No.: US 2025/0120963 A1
(43) Pub. Date: Apr. 17, 2025

(54) METHODS AND COMPOSITIONS FOR THE TREATMENT OF THROMBOCYTOPENIA

(71) Applicant: THE CHILDREN'S MEDICAL CENTER CORPORATION, Boston, MA (US)

(72) Inventors: Roelof H. Bekendam, Roslindale, MA (US); Andrew P. Stone, Brookline, MA (US); Kellie Rae Machlus, Boston, MA (US); Joseph Edward Italiano, JR., Boston, MA (US)

(73) Assignee: THE CHILDREN'S MEDICAL CENTER CORPORATION, Boston, MA (US)

(21) Appl. No.: 18/911,620

(22) Filed: Oct. 10, 2024

Related U.S. Application Data
(60) Provisional application No. 63/543,599, filed on Oct. 11, 2023.

(51) Int. Cl. A61K 31/454; A61K 31/501; A61K 31/55; A61K 45/06; A61P 7/00

(52) U.S. Cl. CPC A61K 31/454; A61K 31/501; A61K 31/55; A61K 45/06; A61P 7/00

(57) ABSTRACT

This disclosure relates to methods and compositions for the treatment of thrombocytopenia.

### CROSS-REFERENCE TO RELATED APPLICATIONS

This application claims benefit under 35 U.S.C. § 119 of U.S. Provisional Application No. 63/543,599 filed on Oct. 11, 2023, the content of which is incorporated herein by reference in its entirety.

### GOVERNMENT SUPPORT

This invention was made with government support under Grant Number HL161175, awarded by the National Institutes of Health. The Government has certain rights in the invention.

### TECHNICAL FIELD

The technology described herein relates to the treatment of thrombocytopenia.

### BACKGROUND

Thrombocytopenia (platelet counts <150×10⁹/L) can be life threatening due to bleeding complications, and is a major clinical problem encountered in multiple conditions, including immune thrombocytopenia purpura (ITP), chemotherapy, surgery, aplastic anemia, human immunodeficiency virus infection, and complications during pregnancy and delivery. It is estimated that approximately 15 million platelet transfusions are administered yearly, with each transfusion costing over $600.

Currently, thrombocytopenia patients refractory to steroids are treated with thrombopoietin (TPO) and TPO mimetics, which take 5 days to increase platelet counts, 12 days to reach maximum effect, and have a host of side effects including venous and arterial thrombosis, increased bone marrow reticulin deposition, and liver toxicity. In certain patients whose marrows contain pre-malignant cells, use of TPO agents can also induce a leukemic transformation TPO mimetics are not ideal because of their high cost, wide range of dose response, and the requirement for lifelong treatment with either weekly injection or oral use with absolute dietary restrictions. Therefore, there is an apparent need for other therapeutic options to treat thrombocytopenia.

### SUMMARY

Inhibition of DNA damage repair is used as a therapeutic approach in cancer treatment. Inhibiting DNA damage repair leads to cell death, particularly among rapidly growing cancer cells. The inventors have now found that accumulation of DNA damage in megakaryocytes (hematopoietic cells that give rise to platelets), has the surprising effect of increasing megakaryopiesis and platelet counts. Accordingly, described herein are methods of treating thrombocytopena through this surprising effect of DNA damage agents and/or DNA damage repair inhibitors.

In one aspect of any of the embodiments, described herein is a method of treating a subject with thrombocytopenia, the method comprising administering to the subject at least one DNA damage reagent and/or at least one DNA damage repair inhibitor. In one aspect of any of the embodiments, described herein is at least one DNA damage reagent and/or at least one DNA damage repair inhibitor for use in a method of treating a subject with thrombocytopenia.

In some embodiments of any of the aspects, the subject is administered a low dose of a DNA damage reagent or a DNA damage repair inhibitor.

In some embodiments of any of the aspects, the DNA damage repair inhibitor is a poly-(ADP-ribose) polymerase (PARP) inhibitor. In some embodiments of any of the aspects, the PARP inhibitor is selected from the group consisting of: niraparib; olaparib; rucaparib; talzaoparib; veliparib; pamiparib; CEP 9722; E7016; iniparib; and 3-aminobenezamide. In some embodiments of any of the aspects, the PARP inhibitor is niraparib, olaparib, or rucaparib. In some embodiments of any of the aspects, the PARP inhibitor is niraparib or rucaparib. In some embodiments of any of the aspects, the PARP inhibitor is niraparib.

In some embodiments of any of the aspects, the subject with thrombocytopenia has or is diagnosed as having a condition selected from the group consisting of: immune thrombocytopenic purpura (ITP), aplastic anemia, human immunodeficiency virus (HIV), acquired immunodeficiency syndrome (AIDS), a myelodysplastic disorder, dehydration, vitamin B12 deficiency, folic acid deficiency, leukemia, myelodysplastic syndrome, liver failure, sepsis, viral infection, bacterial infection, leptospirosis, ACTN1-related thrombocytopenia, amegakaryocytic thrombocytopenia with radio-ulnar synostosis, ANKRD26 related thrombocytopenia, autosomal dominant thrombocytopenia, Bernard-Soulier syndrome, congenital amegakaryocytic thrombocytopenia, congenital amegakaryocytic thrombocytopenia and radioulnar synostosis, CYCS-related thrombocytopenia, Epstein syndrome, ETV6 related thrombocytopenia, Fanconi anemia, Filaminopathies A, FYB related thrombocytopenia, Glanzmann's thrombasthenia, GNE myopathy with congenital thrombocytopenia, Gray platelet syndrome, Harris platelet syndrome, Macrothrombocytopenia, May-Hegglin anomaly, MYH9-related disease, PRKACG-related thrombocytopenia, Paris-Trousseau thrombocytopenia/Jacobsen syndrome, Sebastian syndrome, SLFN14-related thrombocytopenia, Stormorken syndrome, TRPM7-related thrombocytopenia, thrombocytopenia absent radius syndrome, Tropomyosin 4-related thrombocytopenia, TUBB1-related thrombocytopenia, Upshaw-Schulman syndrome, Wiskott-Aldrich syndrome, X-linked thrombocytopenia, X-linked thrombocytopenia with thalassemia, thrombotic thrombocytopenia purpura, hemolytic-uremic syndrome, disseminated intravascular coagulation, paroxysmal nocturnal hemoglobinuria, antiphospholipid syndrome, systemic lupus erythematosus, post-transfusion purpura, neonatal alloimmune thrombocytopenia, hypersplenism, Dengue fever, Gaucher's disease, Zika virus, snakebite, Lyme disease, thrombocytapheresis, and Niemann-Pick disease.

In some embodiments of any of the aspects, the subject with thrombocytopenia has been administered or is being administered a drug selected from the group consisting of: valproic acid, methotrexate, carboplatin, interferon, isotretinoin, panobinostat, a H2 blocker, and a proton-pump inhibitor.

In some embodiments of any of the aspects, the subject is undergoing or selected to undergo a surgical procedure or chemotherapy.

In some embodiments of any of the aspects, the subject is pregnant.

### BRIEF DESCRIPTION OF THE DRAWINGS

FIG. 1 depicts a schematic of genome integrity maintenance.

FIGS. 2A-2B demonstrate that human BM MKs accumulate DNA damage. MKs differentiated from human bone marrow CD34+ cells were stained with antibodies against FIG. 2A) CD41/61-PE, and DAPI to measure ploidy FIG. 2B) Gating on ploidy status reveals a shift from (2N, light gray) to (8N, dark gray) with an increasing level of γH2AX.

FIGS. 3A-3C demonstrate that murine BM MKs have DNA damage that increases with ploidy. FIG. 3A) Immunofluorescent staining of murine femur showing DNA damage (nuclear γH2AX) in MKs sitting against a sinusoid. FIG. 3B) Intracellular flow cytometry of γH2AX in murine bone marrow, n=6, and C) ploidy gated from Lin-CD45+CD41+MK population, n=5.

FIG. 4 depicts graphs of gene expression in human HSCs (left bar), MEPs (middle bar), and MKs (right bar). Data collected from the Normal Human Hematopoiesis database on Bloodspot1 show marked downregulation of key DDR proteins in MKs compared to HSCs and MK/erythroid progenitor cells.

FIGS. 5A-5D demonstrate that niraparib treatment of mice increases platelet and MK counts. Mice were given Niraparib (25 mg/kg, intraperitoneal) or vehicle control for 11 days. FIG. 5A) Platelet counts and immature platelet fraction were measured on a SYSMEX™ hematology analyzer. Line graph: Black circles=Vehicle, gray squares=Niraparib. Bar charts: black=Vehicle, gray=Niraparib. FIG. 5B) MKs and MK-biased HSC counts. Dark gray=DMSO (vehicle), light gray=Niraparib. FIG. 5C) expression of γH2AX. Left bar=control, right bar=Niraparib, and FIG. 5D) MK ploidy was determined by flow cytometry (see FIGS. 9A-9C). n=4-5 mice per group. Gray=Niraparib, black=Vehicle.

FIG. 6 depicts two-photon intravital microscopy images and quantification of MK counts in the calvaria bone marrow of vWF-GFP reporter mice treated with DMSO (dark gray, vehicle) or Niraparib (light gray, 25 mg/kg) for 7 days. Scale bars: 50 um. n=2 mice/group, 4 fields of view/mouse.

FIG. 7 demonstrates that niraparib treatment of mice does not affect platelet function. Platelets were drawn from mice at sacrifice on D11 (see FIG. 6), stimulated with agonists as indicated, and the MFI of activated αIIbβ3 measured by flow cytometry. Black markers=saline, gray markers=Niraparib, 25 mg/kg.

FIG. 8 demonstrates that higher dosage PARP inhibitor treatment does not increase platelet counts. Mice were treated with 50 mg/kg Niraparib or Olaparib daily (i.p.) for 10 or 3 days, respectively, and platelet counts measured by SYSMEX™. Olaparib experiment was terminated early due to severe thrombocytopenia. Black=saline, dark gray=50 mg/kg Niraparib, light gray=50 mg/kg Olaparib.

FIGS. 9A-9C depict flow cytometry gating schematics for identifying/sorting FIG. 9A) Murine bone marrow cells, FIG. 9B) Human CD34+-derived cells, and FIG. 9C) Murine MKs.

FIG. 10 depicts a schematic of CRISPR protocol in CD34+ HSCs. Bottom panel shows example transduction using guides against Itga2b.

FIG. 11 depicts a table of DNA damage agents that are contemplated for treating CD34+ cells and/or MEPs.

FIG. 12 depicts murine BM MK transduced with MSCV-FUCCI. Time course of transition as an MK proceeds through the cell cycle.

FIG. 13A-13B depict representative graphs of platelet aggregation measured after incubation of human platelet rich plasma with PARPi or DMSO vehicle control. FIG. 13A) Aggregation with no agonist FIG. 13B) Aggregation with the effect of Par-1 activating peptide SFLLRN. Black=vehicle, dark gray=Olaparib, medium gray=Niraparib, light gray=Rucaparib.

FIG. 14A-14B demonstrate that PARPi reduce megakaryocyte differentiation in vitro. Lineage depleted bone marrow was cultured with TPO for 4 days in the presence of PARPi or vehicle controls. FIG. 14A) representative images of VWF-GFP signal. FIG. 14B) Mean normalized area of GFP signal (n=3). Error bars±SEM.Circles=Olaparib, squares=Niraparib, triangles=Rucaparib.

FIG. 15A-15B demonstrate megakaryocytes accrue DSBs in vitro and this increases with ploidy. Human BM CD34+ cells differentiated into megakaryocytes in vitro. At Day 10, cells were assayed by flow cytometry for megakaryocyte differentiation (CD41+), ploidy, and DNA damage. FIG. 15A) Ploidy of cells after gating on the CD41/61 positive megakaryocytic population. FIG. 15B) Dot-plot of gH2AX (marker of DSBs) versus CD41/61 with ploidy overlayed demonstrating a concomitant increase in gH2AX signal as ploidy increases.

FIG. 16A-16C demonstrate that mouse megakaryocytes reveal γH2AX foci and thus DSBs. Mouse femur sections cut using the Kawamoto film method. Megakaryocytes and DNA DSBs were immunolabelled with anti-CD41 and anti-gH2AX, respectively. After mounting, slides were imaged on a Zeiss LSM 880 Confocal microscope with a 40× objective. FIG. 16A) Femur with CD41 and gH2AX. FIG. 16B and FIG. 16C) Insets of FIG. 16A where the field of view is 100 μm². gH2AX foci can be seen in megakaryocytes, indicating the presence of DSBs. Approximately 11% of megakaryocytes in this image contained foci.

FIG. 17A-17F demonstrate that Olaparib treatment increases platelet count and alters megakaryocyte polidy in mice. C57B1/6 mice dosed with 50 mg/kg Olaparib intraperitoneally for 5 days consecutive days each week. Dark gray=vehicle, light gray=Olaparib. In both FIG. 17A) 2-week and FIG. 17B) 4-week experiments, we found an increase in platelet count at day 6 before platelet counts returned to approximately baseline. FIG. 17C) After 2-weeks megakaryocyte ploidy shifted to fewer 8N and more 32N cells, whilst after FIG. 17D) 4-weeks an opposite shift to more 8N and fewer 16N cells was identified. The number of megakaryocytes detected by CD41+CD42+ flow cytometric staining of bone marrow cells revealed a trend to fewer megakaryocytes in the Olaparib treatment at both FIG. 17E) 2-weeks and FIG. 17F) 4-weeks.

FIG. 18 demonstrates that PARP inhibition reverses PARylation pattern in megakaryocytes induced by oxidative stress. Lineage depleted murine bone marrow was cultured with TPO for 4 days. Gradient-isolated megakaryocytes subjected to 10 mM of hydrogen peroxide (H₂O₂) in the presence or absence of Olaparib (30 μM or 3 μM). Cells were subsequently lysed in RIPA buffer containing protease inhibitor cocktail. A western blot to detect PARylation was performed. Equal loading was confirmed using an anti-GAPDH antibody. Impage shown is a representative of three experiments.

FIG. 19A-19E detail platelet counts for 25 mg/kg Niraparib. Black=Vehicle, gray=Niraparib. FIG. 19A) Platelet count versus time. FIG. 19B) Platelet count for Vehicle versus Niraparib. FIG. 19C) Percent change in platelet count for Vehicle versus Niraparib. FIG. 19D) Immature Platelet Fraction versus time. FIG. 19E) Mean Platelet Volume versus time.

FIG. 20A-20C detail peripheral blood counts over time. Circles=Vehicle, squares=Niraparib. FIG. 20A) WBC count versus time. FIG. 20B) Neutrophil count versus time. FIG. 20C) Monocyte count versus time.

FIG. 21A-21C demonstrate that Naraparib at 50 mg/kg leads to Pancytopenia. Medium gray=50 mg/kg Niraparib, light gray=50 mg/kg Olaparib. FIG. 21A) Platelet count versus time. FIG. 21B) RBC count versus time. FIG. 21C) WBC count versus time.

FIG. 22A-22H display results of myeloid panel at day 3 HSPC. Significant increase in MKs in both committed progenitors and classical hematopoiesis pathways. Activates emergency and ‘normal’ hematopoiesis. Black=DMSO (vehicle), gray=Niraparib. FIG. 22A) LSK. FIG. 22B) MPP4. FIG. 22C) ST-HSC. FIG. 22D) LT HSC. FIG. 22E) MPP3. FIG. 22F) MPP2. FIG. 22G) CD41+LT-HSC. FIG. 22H) MKs.

FIG. 23A-23H demonstrate day 3 γH2AX positivity in HSPC/Myeloid panel. PARP inhibitor treatment induces DNA damage in MK-committed HSCs and MkPs. White=control, black=Niraparib. FIG. 23A) LT-HSC. FIG. 23B) CD41+HSC. FIG. 23C) MPP2. FIG. 23D) MPP3. FIG. 23E) MPP4. FIG. 23F) ST-HSC. FIG. 23G) MkP. FIG. 23H) CD41+CD42d+.

FIG. 24A-24H demonstrates day 11 HSPC/myeloid panel. Beginning to look like HSC exhaustion although MKs and platelets are still increased. Dark gray=DMSO (vehicle), light gray=Niraparib. FIG. 24A) LSK. FIG. 24B) MPP4. FIG. 24C) ST-HSC. FIG. 24D) LT-HSCs. FIG. 24E) MPP3. FIG. 24F) MPP2. FIG. 24G) CD41+LT-HSC. FIG. 24H) MKs.

FIG. 25 displays day 11 MKs for DMSO and Niraparib, with CD41, CD105 and DAPI staining.

FIG. 26 displays day 11 γH2AX for DMSO and Niraparib.

FIG. 27 displays day 11 ploidy. Black=Vehicle, gray=Niraparib.

FIG. 28 displays day 7 intravital results for DMSO and Niraparib.

FIG. 29 demonstrates human BM MKs accumulate DNA damage. Gating on ploidy status reveals a shift from light gray (2N) to dark gray (8N) with an increasing level of γH2AX.

FIG. 30A-30D demonstrates megakaryocytes have more DNA damage in mice in vivo than other mature lineages. FIG. 30A) Intracellular flow cytometry for γH2AX. FIG. 30B-30C) micrographs with staining for γH2AX, DAPI, CD105 and CD41. FIG. 30D) Micrograph with staining for DAPI, CD41, VE-Cad, γH2AX.

FIG. 31 demonstrates a comet assay detailing double strand breaks in native MKs from murine bone marrow.

FIG. 32A-32H demonstrates DNA damage in MKs scales with ploidy in mice. FIG. 32A) Count versus SYTO 40. Gated on CD45+ Lin-CD41+. FIG. 32B) % γH2AX. FIG. 32C) SSC-A counts for 2n. FIG. 32D) SSC-A counts for 4n. FIG. 32E) SSC-A counts for 8n. FIG. 32F) SSC-A counts for 16n. FIG. 32G) SSC-A counts for 32n. FIG. 32H) SSC-A counts for >32n.

FIG. 33 demonstrates that Niraparib treatment of mice does not affect platelet function. Platelets were drawn from mice at sacrifice on D11, stimulated with agonists as indicated, and the MFI of activated αIIbβ3 measured by flow cytometry. Black=saline, gray=Niraparib (25 mg/kg).

FIG. 34 demonstrates Niraparib treatment in vitro increases ploidy. Black=Vehicle, gray=0.03 μM.

### DETAILED DESCRIPTION

The inventors have surprisingly found that DNA damage accumulation, usually used to induce cell death in tumors, can actually induce the production of platelets and raise platelet counts. Accordingly, in one aspect of any of the embodiments, described herein is a method of treating a subject with thrombocytopenia, the method comprising administering to the subject at least one DNA damage reagent and/or at least one DNA damage repair inhibitor.

As used herein, “DNA damage reagent” refers to an agent which increases the rate or level for DNA damage in a cell or subject by at least 10% or more, e.g. by 10% or more, 50% or more, 100% or more, 200% or more, 500% or more, or 1000% or more. DNA damage includes base lesions, oxidation of bases, alkylation of bases, hydrolysis of bases, bulky adduct formation, base mismatch, monoadduct damage, Di adduct damage, depurination, cross-linking, single or double strand breaks. DNA damage reagents are known in the art and include alkylating agents (e.g., methylnitrosourea, nitrosamines, vinyl chloride), intercalating agents (e.g., ethidium bromide, proflavine, daunorubicin), bromides, sodium azide, psoralen, and benzene. In some embodiments, the DNA damage reagent causes a single-strand break and/or double-strand break.

As used herein “DNA damage repair inhibitor” refers an agent that decreases the amount or rate of at least one DNA damage repair pathway. Multiple DNA damage repair pathways, and the proteins that act in each pathway are known in the art, e.g., base excision repair (BER), microhomology-mediated end-joining (MMEJ), nucleotide excision repair (NER), mismatch repair (MMR), homologous recombination (HR) and non-homologous end joining (NHEJ). See Chatterjee et al. Environ Mol Mutagen 58 (5): 235-263 (2017), which is incorporated by reference herein in its entirety, for further discussion of DNA damage repair pathways. In some embodiments, the DNA damage repair inhibitor is a base excision repair (BER) inhibitor. In some embodiments, the DNA damage repair inhibitor is a microhomology-mediated end-joining (MMEJ) repair inhibitor. In some embodiments, the DNA damage repair inhibitor is a non-homologous end joining (NHEJ) repair inhibitor. In some embodiments, the DNA damage repair inhibitor is an inhibitor of CHK1, CHK2, ATM, ATR, PAXIP, RPA, BRCA1, BRCA2, RAD51, RFC, XRCC1, PCNA, PARP2, PARP, ERCC1, and/or MSH3. In some embodiments, the DNA damage repair inhibitor is an inhibitor of CHK1, CHK2, ATM, ATR, PAXIP, RFC, XRCC1, PCNA, PARP2, and/or PARP. In some embodiments, the DNA damage repair inhibitor is an inhibitor of CHK1, CHK2, ATM, ATR, PARP2, and/or PARP. In some embodiments, the DNA damage repair inhibitor is an inhibitor of PARP.

As used herein, “inhibitor” refers to an agent which can decrease the expression and/or activity of a target, e.g. by at least 10% or more, e.g. by 10% or more, 50% or more, 70% or more, 80% or more, 90% or more, 95% or more, or 98% or more. The efficacy of an inhibitor of one or more targets, e.g. its ability to decrease the level and/or activity of the target can be determined, e.g. by measuring the level of an expression product of the target and/or the activity of the target and/or the level of editing of a system described herein. In some embodiments of any of the aspects, the inhibitor can be an inhibitory nucleic acid; an aptamer; an antibody reagent; an antibody; or a small molecule. An inhibitor of a target described herein can inhibit the activity, expression, or accumulation of the target polypeptide. Inhibitors can include inhibitors that act directly on the target itself (e.g., that bind to the protein or transcript, e.g., direct inhibitors). In some embodiments of any of the aspects, an inhibitor of a specified target is an antibody, antibody reagent, or antigen-binding fragment thereof, that specifically binds to the target.

In some embodiments, the DNA damage repair inhibitor is a poly-(ADP-ribose) polymerase (PARP) inhibitor. PARP (also known as PARP1) has an essential role in facilitating DNA repair. The sequences of PARP are known for a variety of species, e.g, human PARP (see NCBI Gene ID 142).

As used herein “PARP inhibitor” refers to a composition, substance, or molecule that blocks or interferes with the expression or activity of PARP. A variety of different PARP inhibitors are known, including but not limited to talazoparib, veleparib, pamiparib, olaparib, rucaparib and niraparib. Exemplary PARP inhibitors include but are not limited to Iniparib (BSI 201; 4-iodo-3-nitrobenzamide), Olaparib (AZD-2281; KU-59436; 4-[(3-[(4-cyclopropylcarbonyl) piperazin-4-yl]carbonyl)-4-fluorophenyl]methyl (2H) phthalazin-1-one), Rucaparib (AG014699, PF-01367338; 2-{4-[(methylamino)methyl]phenyl}-1,3,4,5-tetrahydro-6H-azepino [5,4,3-cd]indol-6-one), Veliparib (ABT-888; 2-((R)-2-Methylpyrrolidin-2-yl)-1H-benzimidazole-4-carboxamide); CEP-8983; CEP-9722; MK-4827 (Niraparib; 2-{4-[(3S)-Piperidin-3-yl]phenyl}-2H-indazole-7-carboxamide); BMN-673 (LT-673; (8S,9R)-5-fluoro-8-(4-fluorophenyl)-9-(1-methyl-1H-1,2,4-triazol-5-yl)-8,9-dihydro-2H-pyrido[4,3,2-de]phthalazin-3 (7H)-one); LT-674; LT-628; 3-aminobenzamide (INO-1001; 3-AB); PD128763 (3,4-dihydro-5-methyl-1 (2H)-isoquinolinone); NU1025 (8-Hydroxy-2-methyl-4 (3H)-quinazolinone); DR 2313 (1,5,7,8-Tetrahydro-2-methyl-4H-thiopyrano[4,3-d]pyrimidin-4-one); UPF 1069 (5-(2-Oxo-2-phenylethoxy)-3,4-dihydroisoquinolin-1 (2H)-one); EB 47 (5′-Deoxy-5′-[4-[2-[(2,3-Dihydro-1-oxo-1H-isoindol-4-yl)amino]-2-oxoethyl]-1-piperazinyl]-5′-oxoadenosine dihydrochloride); E7016 (Benzopyrano[4,3,2-de]phthalazin-3 (2H)-one, 10-[(4-hydroxy-1-piperidinyl)methyl]-); 4-HQN (4-(1H)-Quinazolinone); ABT-767 and compounds as described in Griffin et al 1998, J. Med. Chem. 41, 5247; Skalitzky et al. 2003, J. Med. Chem. 46:210-213; Zaremba et al. 2007, Anti-Cancer Agents in Medicinal Chemistry 7, 515; Lewis et al. 2007, Curr Opin. Investigational Drugs 8, 1061; Guha 2011, Nature Biotechnology 29, 373-374; Rouleau et al. 2010, Nature Reviews Cancer 10, 293-301; Miknyoczki et al., 2007 Mol Cancer Ther, 6 (8), 2290-2302; Pellicciari et al. 2008, Chem. Med. Chem 3, 91; Jones et al., 2009, J Med Chem, 52 (22), 7170-7185; Mason et al. 2008, Invest New Drugs, 26 (1), 1-5; Ferraris et al., 2010, J. Med. Chem. 53 4561; US patent applications: US2006/0229289; US20070259937; US20120309717; US20130011365; US patents: U.S. Pat. Nos. 8,372,987; 8,362,030; 8,236,802; 8,217,070; 8,183,250; 8,088,760; international patent applications: WO 01/85686; WO 00/42040; WO 00/39070; WO 00/39104; WO 99/11623; WO 99/11628; WO 99/11622; WO 99/59975; WO 99/11644; WO 99/11945; WO 99/11649; and WO 99/59973, each of which is herein incorporated by reference in its entirety. In some embodiments of any of the aspects, the PARP inhibitor is selected from niraparib; olaparib; rucaparib; talzaoparib; veliparib; pamiparib; CEP 9722; E7016; iniparib; and 3-aminobenzamide. In some embodiments of any of the aspects, the PARP inhibitor is niraparib.

The surprising effect of DNA damage agents and/or DNA damage repair inhibitors was found using low doses of DNA damage repair inhibitors. Accordingly, in some embodiments of any of the aspects, the subject is administered a low dose of the at least one DNA damage reagent and/or at least one DNA damage repair inhibitor. In some embodiments of any of the aspects, the subject is administered a low dose of at least one DNA damage repair inhibitor. In some embodiments of any of the aspects, the subject is administered a low dose of at least one PARP inhibitor. In some embodiments of any of the aspects, a low dose is less than 600 mg/day for an adult human subject. In some embodiments of any of the aspects, a low dose is less than 300 mg/day for an adult human subject. In some embodiments of any of the aspects, a low dose is less than 200 mg/day for an adult human subject. In some embodiments of any of the aspects, a low dose is less than 100 mg/day for an adult human subject. In some embodiments of any of the aspects, a low dose is less than 50 mg/day for an adult human subject.

In some embodiments of any of the aspects, a low dose is less than 200 mg/kg. In some embodiments of any of the aspects, a low dose is less than 150 mg/kg. In some embodiments of any of the aspects, a low dose is less than 100 mg/kg. In some embodiments of any of the aspects, a low dose is less than 75 mg/kg. In some embodiments of any of the aspects, a low dose is less than 50 mg/kg. In some embodiments of any of the aspects, a low dose is about 25 mg/kg. In some embodiments of any of the aspects, a low dose is 25 mg/kg.

In some embodiments of any of the aspects, a low dose is defined by the maximal dose administered, e.g., the dose administered before any dosage reductions.

In some embodiments of any of the aspects, the dose administered is 25 mg/kg+10%.

In some embodiments of any of the aspects, a low dose is less than 200 mg/kg/day of niraparib. In some embodiments of any of the aspects, a low dose is less than 150 mg/kg/day of niraparib. In some embodiments of any of the aspects, a low dose is less than 100 mg/kg/day of niraparib. In some embodiments of any of the aspects, a low dose is less than 75 mg/kg. In some embodiments of any of the aspects, a low dose is less than 50 mg/kg/day of niraparib. In some embodiments of any of the aspects, a low dose is about 25 mg/kg/day of niraparib. In some embodiments of any of the aspects, a low dose is 25 mg/kg/day of niraparib. In some embodiments of any of the aspects, a low dose is 25 mg/kg/day+10% of niraparib.

As used herein, “thrombocytopenia” refers to a low platelet count. A low platelet count reduces the ability of a subject to form blood clots and stop bleeding. Thrombocytopenia can arise from a variety of causes, include bone marrow disorders, or the administration of certain medications.

In some embodiments of any of the aspects, the subject with thrombocytopenia has or is diagnosed as having a condition selected from the group consisting of: immune thrombocytopenic purpura (ITP), aplastic anemia, human immunodeficiency virus (HIV), acquired immunodeficiency syndrome (AIDS), a myelodysplastic disorder, dehydration, vitamin B12 deficiency, folic acid deficiency, leukemia, myelodysplastic syndrome, liver failure, sepsis, viral infection, bacterial infection, leptospirosis, ACTN1-related thrombocytopenia, amegakaryocytic thrombocytopenia with radio-ulnar synostosis, ANKRD26 related thrombocytopenia, autosomal dominant thrombocytopenia, Bernard-Soulier syndrome, congenital amegakaryocytic thrombocytopenia, congenital amegakaryocytic thrombocytopenia and radioulnar synostosis, CYCS-related thrombocytopenia, Epstein syndrome, ETV6 related thrombocytopenia, Fanconi anemia, Filaminopathies A, FYB related thrombocytopenia, Glanzmann's thrombasthenia, GNE myopathy with congenital thrombocytopenia, Gray platelet syndrome, Harris platelet syndrome, Macrothrombocytopenia, May-Hegglin anomaly, MYH9-related disease, PRKACG-related thrombocytopenia, Paris-Trousseau thrombocytopenia/Jacobsen syndrome, Sebastian syndrome, SLFN14-related thrombocytopenia, Stormorken syndrome, TRPM7-related thrombocytopenia, thrombocytopenia absent radius syndrome, Tropomyosin 4-related thrombocytopenia, TUBB1-related thrombocytopenia, Upshaw-Schulman syndrome, Wiskott-Aldrich syndrome, X-linked thrombocytopenia, X-linked thrombocytopenia with thalassemia, thrombotic thrombocytopenia purpura, hemolytic-uremic syndrome, disseminated intravascular coagulation, paroxysmal nocturnal hemoglobinuria, antiphospholipid syndrome, systemic lupus erythematosus, post-transfusion purpura, neonatal alloimmune thrombocytopenia, hypersplenism, Dengue fever, Gaucher's disease, Zika virus, snakebite, Lyme disease, thrombocytapheresis, and Niemann-Pick disease.

In some embodiments of any of the aspects, the subject with thrombocytopenia has been administered or is being administered a drug selected from the group consisting of: valproic acid, methotrexate, carboplatin, interferon, isotretinoin, panobinostat, a H2 blocker, and a proton-pump inhibitor. In some embodiments of any of the aspects, the subject with thrombocytopenia has been administered, prior to being administered the at least one DNA damage reagent and/or DNA damage repair inhibitor, a drug selected from the group consisting of: valproic acid, methotrexate, carboplatin, interferon, isotretinoin, panobinostat, a H2 blocker, and a proton-pump inhibitor. In some embodiments of any of the aspects, the subject with thrombocytopenia has been administered or is being administered, within 1 week of being administered the at least one DNA damage reagent and/or DNA damage repair inhibitor, a drug selected from the group consisting of: valproic acid, methotrexate, carboplatin, interferon, isotretinoin, panobinostat, a H2 blocker, and a proton-pump inhibitor. In some embodiments of any of the aspects, the subject with thrombocytopenia has been administered or is being administered, within 2 weeks of being administered the at least one DNA damage reagent and/or DNA damage repair inhibitor, a drug selected from the group consisting of: valproic acid, methotrexate, carboplatin, interferon, isotretinoin, panobinostat, a H2 blocker, and a proton-pump inhibitor. In some embodiments of any of the aspects, the subject with thrombocytopenia has been administered or is being administered, within 1 month of being administered the at least one DNA damage reagent and/or DNA damage repair inhibitor, a drug selected from the group consisting of: valproic acid, methotrexate, carboplatin, interferon, isotretinoin, panobinostat, a H2 blocker, and a proton-pump inhibitor.

In some embodiments of any of the aspects, the subject is undergoing or selected to undergo a surgical procedure or chemotherapy. In some embodiments of any of the aspects, the subject with thrombocytopenia has been administered chemotherapy or undergone a surgical procedure, within 1 month prior to being administered the at least one DNA damage reagent and/or DNA damage repair inhibitor. In some embodiments of any of the aspects, the subject with thrombocytopenia has been administered chemotherapy or undergone a surgical procedure, within 2 weeks prior to being administered the at least one DNA damage reagent and/or DNA damage repair inhibitor. In some embodiments of any of the aspects, the subject with thrombocytopenia has been administered chemotherapy or undergone a surgical procedure, within 1 week prior to being administered the at least one DNA damage reagent and/or DNA damage repair inhibitor.

In some embodiments of any of the aspects, the subject with thrombocytopenia has been selected or scheduled to be administered chemotherapy or undergo a surgical procedure, within 1 week after being administered the at least one DNA damage reagent and/or DNA damage repair inhibitor. In some embodiments of any of the aspects, the subject with thrombocytopenia has been selected or scheduled to be administered chemotherapy or undergo a surgical procedure, within 2 weeks after being administered the at least one DNA damage reagent and/or DNA damage repair inhibitor. In some embodiments of any of the aspects, the subject with thrombocytopenia has been selected or scheduled to be administered chemotherapy or undergo a surgical procedure, within 1 month after being administered the at least one DNA damage reagent and/or DNA damage repair inhibitor.

In some embodiments of any of the aspects, the method further comprises administering chemotherapy or a surgical procedure, within 1 week after the subject is administered the at least one DNA damage reagent and/or DNA damage repair inhibitor. In some embodiments of any of the aspects, the method further comprises administering chemotherapy or a surgical procedure, within 2 weeks after the subject is administered the at least one DNA damage reagent and/or DNA damage repair inhibitor. In some embodiments of any of the aspects, the method further comprises administering chemotherapy or a surgical procedure, within 1 month after the subject is administered the at least one DNA damage reagent and/or DNA damage repair inhibitor.

In some embodiments of any of the aspects, the subject is pregnant.

In some embodiments, the methods described herein relate to treating a subject having or diagnosed as having thrombocytopena the at least one DNA damage reagent and/or DNA damage repair inhibitor. Subjects having thrombocytopenia can be identified by a physician using current methods of diagnosing thrombocytopenia. Symptoms and/or complications of thrombocytopenia which characterize these conditions and aid in diagnosis are well known in the art and include but are not limited to, easy or excessive bruising, superficial bleeding, prolonged bleeding, bleeding gums or nose, blood in urine or stool, unusually heavy menstrual flow, fatigue, and enlarged spleen. Tests that may aid in a diagnosis of, e.g. thrombocytopenia include, but are not limited to, blood tests A family history of thrombocytopenia, or exposure to risk factors for thrombocytopenia (e.g. taking certain medications) can also aid in determining if a subject is likely to have thrombocytopenia or in making a diagnosis of thrombocytopenia.

The compositions and methods described herein can be administered to a subject having or diagnosed as having thrombocytopenia. In some embodiments, the methods described herein comprise administering an effective amount of compositions described herein, e.g. an at least one DNA damage reagent and/or DNA damage repair inhibitor to a subject in order to alleviate a symptom of thrombocytopenia. As used herein, “alleviating a symptom” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, topical, injection, or intratumoral administration. Administration can be local or systemic.

The term “effective amount” as used herein refers to the amount of the at least one DNA damage reagent and/or DNA damage repair inhibitor needed to alleviate at least one or more symptom of the disease or disorder, and relates to a sufficient amount of pharmacological composition to provide the desired effect. The term “therapeutically effective amount” therefore refers to an amount of the at least one DNA damage reagent and/or DNA damage repair inhibitor that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the at least one DNA damage reagent and/or DNA damage repair inhibitor, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for platelet levels, among others. The dosage can be determined by a physician and adjusted, as necessary, to suit observed effects of the treatment.

In some embodiments, the technology described herein relates to a pharmaceutical composition comprising the at least one DNA damage reagent and/or DNA damage repair inhibitor as described herein, and optionally a pharmaceutically acceptable carrier. In some embodiments, the active ingredients of the pharmaceutical composition comprise the at least one DNA damage reagent and/or DNA damage repair inhibitor as described herein. In some embodiments, the active ingredients of the pharmaceutical composition consist essentially of the at least one DNA damage reagent and/or DNA damage repair inhibitor as described herein. In some embodiments, the active ingredients of the pharmaceutical composition consist of the at least one DNA damage reagent and/or DNA damage repair inhibitor as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. the at least one DNA damage reagent and/or DNA damage repair inhibitor as described herein.

In some embodiments, the pharmaceutical composition comprising the at least one DNA damage reagent and/or DNA damage repair inhibitor as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of the at least one DNA damage reagent and/or DNA damage repair inhibitor as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt of an agent as disclosed herein can also be incorporated into the parenteral dosage forms of the disclosure, including conventional and controlled-release parenteral dosage forms.

Pharmaceutical compositions comprising the at least one DNA damage reagent and/or DNA damage repair inhibitor can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the at least one DNA damage reagent and/or DNA damage repair inhibitor can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the at least one DNA damage reagent and/or DNA damage repair inhibitor described herein is administered as a monotherapy, e.g., another treatment for the thrombocytopenia is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include blood transfusion, platelet transfection, corticosteroids, immune suppressants, splenectomy, and plasma exchange.

In certain embodiments, an effective dose of a composition comprising the at least one DNA damage reagent and/or DNA damage repair inhibitor as described herein can be administered to a patient once. In certain embodiments, an effective dose of a composition comprising the at least one DNA damage reagent and/or DNA damage repair inhibitor can be administered to a patient repeatedly. For systemic administration, subjects can be administered a therapeutic amount of a composition comprising the at least one DNA damage reagent and/or DNA damage repair inhibitor, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more. In some embodiments of any of the aspects, the methods described herein increase platelet levels in the subject.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active ingredient(s). The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising the at least one DNA damage reagent and/or DNA damage repair inhibitor can be administered over a period of time, such as over a 5 minute, 10 minute, 15 minute, 20 minute, or 25 minute period.

The dosage ranges for the administration of the at least one DNA damage reagent and/or DNA damage repair inhibitor, according to the methods described herein depend upon, for example, the form of the at least one DNA damage reagent and/or DNA damage repair inhibitor, its potency, and the extent to which symptoms, markers, or indicators of a condition described herein are desired to be reduced, for example the percentage to which, for example, platelet levels are desired to be increased. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of the at least one DNA damage reagent and/or DNA damage repair inhibitor in, e.g. the treatment of a condition described herein, or to induce a response as described herein (e.g. platelet level increases) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. platelet levels. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. platelet levels). It is well within the ability of one skilled in the art to monitor efficacy of administration and/or treatment by measuring any one of such parameters, or any combination of parameters. Efficacy can be assessed in animal models of a condition described herein, for example treatment of a murine model of thrombocytopenia. When using an experimental animal model, efficacy of treatment is evidenced when a statistically significant change in a marker is observed, e.g. platelet levels.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomologus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of thrombocytopenia. A subject can be male or female.

A subject can be one who has been previously diagnosed with or identified as suffering from or having a condition in need of treatment (e.g. thrombocytopenia) or one or more complications related to such a condition, and optionally, have already undergone treatment for thrombocytopenia or the one or more complications related to thrombocytopenia Alternatively, a subject can also be one who has not been previously diagnosed as having thrombocytopenia or one or more complications related to thrombocytopenia. For example, a subject can be one who exhibits one or more risk factors for thrombocytopenia or one or more complications related to thrombocytopenia or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (mRNA) or antisense RNA derived from a nucleic acid fragment or fragments of the invention and/or to the translation of mRNA into a polypeptide.

“Operably linked” refers to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, the at least one DNA damage reagent and/or DNA damage repair inhibitor described herein is exogenous. In some embodiments of any of the aspects, the at least one DNA damage reagent and/or DNA damage repair inhibitor described herein is ectopic. In some embodiments of any of the aspects, the at least one DNA damage reagent and/or DNA damage repair inhibitor described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

In some embodiments, a nucleic acid as described herein is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

In some embodiments of any of the aspects, the vector or nucleic acid described herein is codon-optomized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle may be utilized for the purpose of transferring any nucleic acids into cells either in vitro or in vivo. Numerous forms of viral vectors are known in the art.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

In some embodiments of any of the aspects, an inhibitor is an inhibitory nucleic acid. In some embodiments of any of the aspects, inhibitors of the expression of a given gene can be an inhibitory nucleic acid. As used herein, “inhibitory nucleic acid” refers to a nucleic acid molecule which can inhibit the expression of a target, e.g., double-stranded RNAs (dsRNAs), inhibitory RNAs (iRNAs), and the like. In some embodiments of any of the aspects, the inhibitory nucleic acid can be a silencing RNA (siRNA), microRNA (miRNA), or short hairpin RNA (shRNA). Inhibitory nucleic acids can also include guide sequence molecules (e.g., a guide RNA) that function, e.g., in combination with an enzyme, to induce insertions, deletions, indels, and/or mutations of a target, thereby inhibiting the expression of the target.

In some embodiments of any of the aspects, an iNA comprises a sequence that is complementary to at least a portion of a target sequence described herein. In some embodiments of any of the aspects, an iNA comprises a sequence at least 15 nucleotides in length that is complementary to at least a portion of a target sequence described herein. In some embodiments of any of the aspects, an iNA comprises a sequence at least 20 nucleotides in length that is complementary to at least a portion of a target sequence described herein.

In some embodiments of any of the aspects, an iNA comprises a sequence that is the reverse complement to at least a portion of a target sequence described herein. In some embodiments of any of the aspects, an iNA comprises a sequence at least 15 nucleotides in length that is the reverse complement to at least a portion of a target sequence described herein. In some embodiments of any of the aspects, an iNA comprises a sequence at least 20 nucleotides in length that is the reverse complement to at least a portion of a target sequence described herein.

In some embodiments of any of the aspects, an iNA comprises a sequence that can specifically hybridize to at least a portion of a target sequence described herein. In some embodiments of any of the aspects, an iNA comprises a sequence at least 15 nucleotides in length that can specifically hybridize to at least a portion of a target sequence described herein. In some embodiments of any of the aspects, an iNA comprises a sequence at least 20 nucleotides in length that can specifically hybridize to at least a portion of a target sequence described herein.

Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA (or modified nucleic acids as described below herein) and which mediates the targeted cleavage of an RNA transcript via an RNA-induced silencing complex (RISC) pathway. In some embodiments of any of the aspects, an iRNA as described herein effects inhibition of the expression and/or activity of a target, e.g. PARP. In some embodiments of any of the aspects, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA. In some embodiments of any of the aspects, administering an inhibitor (e.g. an iRNA) to a subject results in a decrease in the target mRNA level in the subject by at least about 5%, about 10%, about 20%, about 30%, about 40%, about 50%, about 60%, about 70%, about 80%, about 90%, about 95%, about 99%, up to and including 100% of the target mRNA level found in the subject without the presence of the iRNA.

In some embodiments of any of the aspects, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target, e.g., it can span one or more intron boundaries. The other strand (the sense strand) includes a region that is complementary to the antisense strand, such that the two strands hybridize and form a duplex structure when combined under suitable conditions. Generally, the duplex structure is between 15 and 30 base pairs in length inclusive, more generally between 18 and 25 base pairs in length inclusive, yet more generally between 19 and 24 base pairs in length inclusive, and most generally between 19 and 21 base pairs in length, inclusive. Similarly, the region of complementarity to the target sequence is between 15 and 30 base pairs in length inclusive, more generally between 18 and 25 base pairs in length inclusive, yet more generally between 19 and 24 base pairs in length inclusive, and most generally between 19 and 21 base pairs in length nucleotides in length, inclusive. In some embodiments of any of the aspects, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

Exemplary embodiments of types of inhibitory nucleic acids can include, e.g., siRNA, shRNA, miRNA, and/or amiRNA, which are well known in the art. One skilled in the art would be able to design further siRNA, shRNA, or miRNA to target the nucleic acid sequence of, e.g., PARP, e.g., using publically available design tools. siRNA, shRNA, or miRNA is commonly made using companies such as Dharmacon (Layfayette, CO) or Sigma Aldrich (St. Louis, MO).

In some embodiments of the various aspects described herein, the inhibitory nucleic acid is a guide nucleic acid (gNA). As used herein, the terms “guide nucleic acid,” “guide sequence,” “crRNA,” “guide RNA,” “single guide RNA,” “gRNA” or “CRISPR guide sequence” refer to a nucleic acid comprising a sequence that determines the specificity of an enzyme, e.g., the Cas DNA binding protein of a CRISPR/Cas system, to a polynucleotide target. The gNA can comprise a polynucleotide sequence with at least partial complementarity with a target nucleic acid sequence, sufficient to hybridize with the target nucleic acid sequence and to direct sequence-specific binding of an enzyme, e.g, a nuclease, to the target nucleic acid sequence.

In some embodiments, the enzyme directed by the gNA is a gene-editing protein, e.g., any nuclease that induces a nick or double-strand break into a desired recognition site. Such enzymes can be native or engineered. These breaks can then be repaired by the cell in one of two ways: non-homologous end joining and homology-directed repair (homologous recombination). In non-homologous end joining (NHEJ), the double-strand breaks are repaired by direct ligation of the break ends to one another. As such, no new nucleic acid material is inserted into the site, although some nucleic acid material may be lost, resulting in a deletion. In homology-directed repair, a donor polynucleotide with homology to the cleaved target DNA sequence can be used as a template for repair of the cleaved target DNA sequence, resulting in the transfer of genetic information from the donor polynucleotide to the target DNA. Therefore, new nucleic acid material may be inserted/copied into the site. The modifications of the target DNA due to NHEJ and/or homology-directed repair can be used for gene correction, gene replacement, gene tagging, transgene insertion, nucleotide deletion, gene disruption, gene mutation, etc.

In one embodiment, the gene-editing protein is a CRISPR-associated nuclease. The native prokaryotic CRISPR-associated nuclease system comprises an array of short repeats with intervening variable sequences of constant length (i.e., clusters of regularly interspaced short palindromic repeats), and CRISPR-associated (“Cas”) nuclease proteins. The RNA of the transcribed CRISPR array is processed by a subset of the Cas proteins into small guide RNAs, which generally have two components as discussed below. There are at least three different systems: Type I, Type II and Type III. The enzymes involved in the processing of the RNA into mature crRNA are different in the 3 systems. In the native prokaryotic system, the guide RNA (“gRNA”) comprises two short, non-coding RNA species referred to as CRISPR RNA (“crRNA”) and trans-acting RNA (“tracrRNA”). In an exemplary system, the gRNA forms a complex with a nuclease, for example, a Cas nuclease. The gRNA: nuclease complex binds a target polynucleotide sequence having a protospacer adjacent motif (“PAM”) and a protospacer, which is a sequence complementary to a portion of the gRNA. The recognition and binding of the target polynucleotide by the gRNA: nuclease complex induces cleavage of the target.

Any CRISPR-associated nuclease can be used in the system and methods of the invention. CRISPR nuclease systems are known to those of skill in the art, e.g. Cas9, Cas12, Cas12a, or the like, see Patents/applications 8,993,233, US 2015/0291965, US 2016/0175462, US 2015/0020223, US 2014/0179770, 8,697,359; 8,771,945; 8,795,965; WO 2015/191693; U.S. Pat. No. 8,889,418; WO 2015/089351; WO 2015/089486; WO 2016/028682; WO 2016/049258; WO 2016/094867; WO 2016/094872; WO 2016/094874; WO 2016/112242; US 2016/0153004; US 2015/0056705; US 2016/0090607; US 2016/0029604; 8,865,406; 8,871,445; each of which are incorporated by reference in their entirety. The nuclease can also be a phage Cas nuclease, e.g., CasΦ (e.g., Pausch et al. Science 369:333-7 (2020); which is incorporated by reference herein in its entirety).

The full-length guide nucleic acid strand can be any length. For example, the guide nucleic acid strand can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments of the various aspects described herein, a nucleic acid strand is less than about 75, 50, 45, 40, 35, 30, 25, 20, 15, 12, or fewer nucleotides in length. For example, the guide nucleic acid sequence is 10-30 nucleotides long.

In addition to a sequence that is complementary to a target nucleic acid, in some embodiments, the gNA also comprises a scaffold sequence. Expression of a gNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gNA may be referred to as a single guide RNA (sgRNA).

In some embodiments of the various aspects described herein, the guide nucleic acid is designed using a guide design tool (e.g., Benchling™; Broad Institute GPP™; CasOFFinder™; CHOPCHOP™; CRISPOR™; Deskgen™; E-CRISP™; Geneious™; GenHub™; GUIDES™ (e.g., for library design); Horizon Discovery™; IDT™; Off-Spotter™; and Synthego™; which are available on the world wide web).

In some embodiments of any of the aspects, the RNA of an iRNA, e.g., a dsRNA, is chemically modified to enhance stability or other beneficial characteristics. The nucleic acids described herein may be synthesized and/or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In some embodiments of any of the aspects, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; others having mixed N, O, S and CH2 component parts, and oligonucleosides with heteroatom backbones, and in particular —CH2-NH—CH2-, —CH2-N(CH3)-O—CH2-[known as a methylene (methylimino) or MMI backbone], —CH2- O—N(CH3)-CH2-, —CH2-N(CH3)-N(CH3)-CH2- and —N(CH3)-CH2-CH2-[wherein the native phosphodiester backbone is represented as —O—P—O—CH2-].

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33 (1): 439-447; Mook, O R. et al., (2007) Mol Canc Ther 6 (3): 833-843; Grunweller, A. et al., (2003) Nucleic Acids Research 31 (12): 3185-3193).

Modified RNAs can also contain one or more substituted sugar moieties. The iRNAs, e.g., dsRNAs, described herein can include one of the following at the 2′ position: OH; F; O—, S—, or N-alkyl; O—, S—, or N-alkenyl; O—, S- or N-alkynyl; or O-alkyl-O-alkyl, wherein the alkyl, alkenyl and alkynyl may be substituted or unsubstituted C₁ to C10 alkyl or C₂ to C10 alkenyl and alkynyl. Exemplary suitable modifications include O[(CH2)nO]mCH3, O(CH2)·nOCH3, O(CH2)nNH2, O(CH2)nCH3, O(CH2)nONH2, and O(CH2)nON[(CH2)nCH3)]2, where n and m are from 1 to about 10. In some embodiments of any of the aspects, dsRNAs include one of the following at the 2′ position: C1 to C10 lower alkyl, substituted lower alkyl, alkaryl, aralkyl, O-alkaryl or O-aralkyl, SH, SCH3, OCN, Cl, Br, CN, CF3, OCF3, SOCH3, SO2CH3, ONO2, NO2, N3, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments of any of the aspects, the modification includes a 2′ methoxyethoxy (2′-O—CH2CH2OCH3, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH2) 2ON(CH3) 2 group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH2-O—CH2-N(CH2) 2, also described in examples herein below.

Other modifications include 2′-methoxy (2′-OCH3), 2′-aminopropoxy (2′-OCH2CH2CH2NH2) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar.

An inhibitory nucleic acid can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Certain of these nucleobases are particularly useful for increasing the binding affinity of the inhibitory nucleic acids featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

The preparation of the modified nucleic acids, backbones, and nucleobases described above are well known in the art.

Another modification of an inhibitory nucleic acid featured in the invention involves chemically linking to the inhibitory nucleic acid to one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86:6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937).

Antibody reagents specific for the targets and/or markers described herein, e.g., PARP are known in the art. For example, such reagents are readily commercially available, e.g., the anti-PARP antibodies 123 (Invitrogen, Cat No. 436400), C-2-10 (Invitrogen, Cat No MA3-950), C.384.8 (Invitrogen, Waltham MA, Cat. No. MA5-15031), SU03-68 (Invitrogen Ca No. MA5-41125), EPR18461 (Abcam, Cambridge, UK, Cat. No. ab191217), and E102 (Abcam Cat. No. ab32138).

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. thrombocytopenia. The term “treating” includes reducing or alleviating at least one adverse effect or symptom of a condition, disease or disorder associated with thrombocytopenia. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

In some embodiments of any of the aspects, treatment is measured by platelet levels, e.g, treatment occurs if the subject's platelet levels increase.

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to uncontrolled bleeding, surgery, need for a transfusion, or internal bleeding. In some embodiments of any of the aspects, prophylactic treatment is not prevention of all symptoms or signs of a disease.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “nanoparticle” refers to particles that are on the order of about 1 to 1,000 nanometers in diameter or width. The term “nanoparticle” includes nanospheres; nanorods; nanoshells; and nanoprisms; these nanoparticles may be part of a nanonetwork. The term “nanoparticles” also encompasses liposomes and lipid particles having the size of a nanoparticle. Exemplary nanoparticles include lipid nanoparticles or ferritin nanoparticles. Lipid nanoparticles can comprise multiple components, including, e.g., ionizable lipids (such as MC3, DLin-MC3-DMA, ALC-0315, or SM-102), pegylated lipids (such as PEG2000-C-DMG, PEG2000-DMG, ALC-0159), phospholipids (such as DSPC), and cholesterol.

Exemplary liposomes can comprise, e.g., DSPC, DPPC, DSPG, Cholesterol, hydrogenated soy phosphatidylcholine, soy phosphatidyl choline, methoxypolyethylene glycol (mPEG-DSPE) phosphatidyl choline (PC), phosphatidyl glycerol (PG), distearoylphosphatidylcholine, and combinations thereof.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, perfusion, injection, or other delivery method well known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:
1. A method of treating a subject with thrombocytopenia, the method comprising administering to the subject at least one DNA damage reagent and/or at least one DNA damage repair inhibitor.
2. The method of paragraph 1, comprising administering to the subject a low dose of a DNA damage reagent or a DNA damage repair inhibitor.
3. The method of any one of the preceding paragraphs, wherein the DNA damage repair inhibitor is a poly-(ADP-ribose) polymerase (PARP) inhibitor.
4. The method of any one of the preceding paragraphs, wherein the PARP inhibitor is selected from the group consisting of:
   niraparib; olaparib; rucaparib; talzaoparib; veliparib; pamiparib; CEP 9722; E7016;
   iniparib; and 3-aminobenezamide.
5. The method of any one of the preceding paragraphs, wherein the PARP inhibitor is niraparib, olaparib, or rucaparib.
6. The method of any one of the preceding paragraphs, wherein the PARP inhibitor is niraparib or rucaparib.
7. The method of any one of the preceding paragraphs, wherein the PARP inhibitor is niraparib.
8. The method of any one of the preceding paragraphs, wherein the subject with thrombocytopenia has or is diagnosed as having a condition selected from the group consisting of:
   immune thrombocytopenia purpura (ITP), aplastic anemia, human immunodeficiency virus (HIV), acquired immunodeficiency syndrome (AIDS), a myelodysplastic disorder, dehydration, vitamin B12 deficiency, folic acid deficiency, leukemia, myelodysplastic syndrome, liver failure, sepsis, viral infection, bacterial infection, leptospirosis, ACTN1-related thrombocytopenia, amegakaryocytic thrombocytopenia with radio-ulnar synostosis, ANKRD26 related thrombocytopenia, autosomal dominant thrombocytopenia, Bernard-Soulier syndrome, congenital amegakaryocytic thrombocytopenia, congenital amegakaryocytic thrombocytopenia and radioulnar synostosis, CYCS-related thrombocytopenia, Epstein syndrome, ETV6 related thrombocytopenia, Fanconi anemia, Filaminopathies A, FYB related thrombocytopenia, Glanzmann's thrombasthenia, GNE myopathy with congenital thrombocytopenia, Gray platelet syndrome, Harris platelet syndrome, Macrothrombocytopenia, May-Hegglin anomaly, MYH9-related disease, PRKACG-related thrombocytopenia, Paris-Trousseau thrombocytopenia/Jacobsen syndrome, Sebastian syndrome, SLFN14-related thrombocytopenia, Stormorken syndrome, TRPM7-related thrombocytopenia, thrombocytopenia absent radius syndrome, Tropomyosin 4-related thrombocytopenia, TUBB1-related thrombocytopenia, Upshaw-Schulman syndrome, Wiskott-Aldrich syndrome, X-linked thrombocytopenia, X-linked thrombocytopenia with thalassemia, thrombotic thrombocytopenia purpura, hemolytic-uremic syndrome, disseminated intravascular coagulation, paroxysmal nocturnal hemoglobinuria, antiphospholipid syndrome, systemic lupus erythematosus, post-transfusion purpura, neonatal alloimmune thrombocytopenia, hypersplenism, Dengue fever, Gaucher's disease, Zika virus, snakebite, Lyme disease, thrombocytapheresis, and Niemann-Pick disease.
9. The method of any one of the preceding paragraphs, wherein the subject with thrombocytopenia has been administered or is being administered a drug selected from the group consisting of:
   valproic acid, methotrexate, carboplatin, interferon, isotretinoin, panobinostat, a H2 blocker, and a proton-pump inhibitor.
10. The method of any one of the preceding paragraphs, wherein the subject is undergoing or selected to undergo a surgical procedure or chemotherapy.
11. The method of any one of the preceding paragraphs, wherein the subject is pregnant.

The technology described herein is further illustrated by the following examples which in no way should be construed as being further limiting.

### EXAMPLES

### Example 1

Described herein are methods of manipulating DNA damage repair inhibition to increase megakaryopoiesis and increase platelet production. Low dose, targeted DNA damage induction/DNA damage repair inhibition can increase platelet counts in patients with thrombocytopenia disorders (e.g. immune thrombocytopenia, myelodysplastic disorders).

Megakaryocytes are large hematopoietic cells that can reach ploidy levels greater than 64n. Polyploid cells are more susceptible to DNA damage, which raises the potential of DNA damage accumulation in polyploid megakaryocytes. Currently, the only class of DNA damage repair inhibitor drugs that are FDA approved are poly(ADP-ribose) polymerase (PARP) inhibitors in the solid tumor space. They exhibit their anti-tumor effect through induction of single stranded DNA breaks, ultimately leading to double stranded DNA breaks causing cell death. Hematological side effects, including thrombocytopenia, are the main dose-limiting toxicity in the clinic. The inventors investigated mechanisms by which PARP inhibitors cause thrombocytopenia. The data shows that high doses of niraparib, a clinically approved PARP inhibitor, leads to pancytopenia in mice through bone marrow underproduction by induction of cell death. Unexpectedly, administration of lower doses of niraparib caused an increase in megakaryopoiesis and a significant increase in the mouse platelet count within 7 days of initial administration. Increased DNA damage accumulation in megakaryocytes from the niraparib group was confirmed with immunofluorescence staining on munne long bones using γH2AX, a marker of DNA damage.

Similarly, these findings were corroborated in vitro, where lineage depleted bone marrow cells from WT mice show increased megakaryocyte maturation at lower doses of niraparib and decreased maturation at higher doses. Overall, the data shows that DNA damage repair inhibition/induction of DNA damage leads to a skew towards more platelet generating megakaryocytes. The inventors therefore contemplate methods of treating patients suffering from thrombocytopenia states that require a fast increase in their platelet count (e.g. prior to a planned procedure).

Background: Megakaryocytes are large hematopoietic cells that can reach ploidy levels greater than 64n. The potential for DNA damage accumulation in polyploid megakaryocytes is of interest as cancer cells with a DNA complement of even 4N are more susceptible to DNA damage. Poly(ADP-ribose) polymerase (PARP) inhibitors exhibit anti-tumor effects in solid tumors through induction of single stranded DNA breaks, ultimately leading to double stranded breaks and cell death. Hematological side effects including thrombocytopenia are the main dose-limiting toxicity in the clinic. However, the molecular mechanisms behind this thrombocytopenia remain largely unknown.

Aims Investigate the potential for DNA damage accumulation in megakaryocytes and understand the effect of PARP inhibitor-induced thrombocytopenia by assessing megakaryocytic function and platelet production.

Methods: Immunofluorescence staining of γH2AX, a marker of DNA damage, was performed on native and in-vitro matured munne and human megakaryocytes. The effect of three clinically relevant PARP inhibitors was assessed on platelet functional assays and in vitro megakaryopoiesis. Finally, PARP inhibitors were administered to mice to investigate how they affect megakaryocyte differentiation, polyploidization, and platelet formation.

Results: Analysis of wildtype murine femurs revealed that megakaryocytes were positive for γH2AX staining. Human megakaryocytes, differentiated from bone marrow derived CD34+ cells, similarly showed γH2AX foci. Increased megakaryocyte γH2AX was associated with higher ploidy. Addition of PARP inhibitors to munne lineage depleted bone marrow cells resulted in reduced megakaryopoiesis and decreased megakaryocyte PARylation. Platelet function, however, remained intact. In vivo application of PARP inhibitors to healthy C57BL/6J mice led to a significant shift toward higher ploidy megakaryocytes (>32n).

Discussion: This data provides evidence that higher ploidy correlates with increased DNA damage and PARP inhibitors can exert their hematological toxicity through increasing DNA damage to megakaryocytes.

### Example 2

Platelet levels are critical to hemostasis and are therefore tightly regulated in healthy individuals. In several pathologies, the disruption of thrombopoiesis (platelet production) leads to reduced platelet counts which significantly increase bleeding risks. Thrombocytopenia occurs when platelet counts drop to dangerously low levels (<150×109/L), which can be life-threatening due to a heightened risk of bleeding. Thrombocytopenia is encountered in multiple conditions, including immune thrombocytopenia (ITP), chemotherapy, surgery, aplastic anemia, human immunodeficiency virus infection, and during pregnancy and delivery. Unfortunately, effective, durable treatment with steroids for ITP is only achieved in 10-30% patients. 2 These patients are often treated with thrombopoietin (TPO) mimetics which take 5 days to increase platelet counts with optimal effect at day 12, or Rituximab, which can take 4-6 weeks for peak results. In addition, TPO mimetics can induce serious side effects including venous/arterial thrombosis, increased bone marrow reticulin deposition, liver toxicity, and myelofibrosis. 3,4 Further, TPO mimetics are poor therapeutics due to their high cost, wide range of dose responses, and the requirement for lifelong treatment with either weekly injection or oral use with stringent dietary restrictions. 4 Additionally, approximately 1.3 million platelet transfusions are administered yearly for indications such as trauma or chemotherapy-induced thrombocytopenia with each platelet unit costing ˜$1300, and the average treatment cost per patient being over $3400.5,6 Due to our continued lack of understanding about the fundamental processes that drive megakaryocyte (MK) differentiation and proplatelet production, there are no other therapeutic alternatives. Therefore, a better understanding of the biology of MK differentiation and maturation is essential to develop more diverse therapeutic options to treat thrombocytopenia.

MKs are large, polyploid, hematopoietic cells that reside primarily in the bone marrow and release platelets into circulation. One of the unique and defining features of MKs is their large polyploid nucleus contained within a single nuclear membrane. The field currently has an insufficient understanding of the impact that having this sheer volume of DNA (up to 128N) within a single cell has on its physiology. Further, our knowledge of the transcriptional landscape of these cells and how they respond to acquiring many-fold more DNA than other mature blood cells has lagged. Equally, while there are other polyploid cells including muscle, hepatocytes, and placenta, these cells are multinucleate, making MKs unique in their accumulation of multiple copies of the genome contained within one, multilobed nucleus. Being as such, all intra-nuclear signaling occurs within the same nuclear organelle. As accumulation of DNA damage is a major determinant of cellular function in different cell types7 the inventors set out to characterize the relationship between DNA accumulation, DNA instability, MK development, and platelet production.

Unexpectedly, the inventors discovered extensive DNA damage accumulation in both primary human and murine MKs in normal physiology. Using nuclear γH2AX as a measure of DNA damage, it was found that human and murine bone marrow MKs had more DNA damage compared to other differentiated bone marrow cells. Further, the data revealed a significant, positive correlation of DNA damage levels with increasing ploidy, with almost 100% of 32N MKs showing γH2AX positivity, indicating rapid accumulation of damage with each endomitotic cycle. The mechanisms and consequences of DNA damage accumulation in MK progenitors and MKs will be determined as described below.

In addition to normal physiology, it was also examined if increasing amounts of DNA lesions could enhance MK production. Mice were treated with the poly-ADP ribose polymerase (PARP) inhibitor Niraparib, which leads to accumulation of DNA damage in hematopoietic stem and progenitor cells (HSPCs). 8 Niraparib treatment led to a significant increase in both MK number and platelet counts, with platelets reaching over of 150% of normal levels. Elevated MK and platelet counts were accompanied by significantly increased DNA damage in MK progenitors and enhanced MK ploidy. These results directly linked increased accumulation of DNA damage in MK progenitors to elevated ploidy and increased platelet production in vivo. Notably, high concentrations of PARP inhibitors in vivo and in vitro lead to accrued DNA damage that induced MK death and thrombocytopenia, suggesting that there may be a ‘goldilocks effect’ where there is an optimal treatment between the extremes of untreated and induction of thrombocytopenia. Indeed, these results are in line with previously reported data showing that PARP inhibitors at specific dosages can cause thrombocytopenia9, and that ablation of the DNA repair gene FANCA impairs platelet formation, indicating that high levels of DNA damage are lethal to MKs. 10

How does induction of moderate amounts of DNA damage increase MK differentiation? It has been previously shown that MEPs with a slower cell cycle differentiate into MKs at the expense of erythroid cells. 11 As unrepaired DNA lesions stall cell cycle progression at the G1/S transition, it was hypothesized that DNA damage accumulation mediates this cell cycle stalling and may preferentially bias cells along the MK lineage; this hypothesis is tested as described below. Together, the data indicate that MKs accumulate DNA damage over maturation, and that the controlled accumulation of DNA lesions can regulate the number of MKs and subsequent platelets both in vitro and in vivo. These findings are the first to explore the functional impact of DNA damage in MKs and will accelerate our understanding of the drivers of MK development and triggers for platelet production. The findings described herein provide therapeutic strategies to mitigate or treat the effects of disorders where platelet counts are reduced.

### Background

Mechanisms of megakaryopoiesis. Megakaryopoiesis is the process by which MKs develop from hematopoietic stem cells (HSCs) along the myeloid branch of hematopoiesis. HSCs differentiate into MKs under the direction of TPO, which is constitutively produced in the liver. TPO signaling through its receptor myeloproliferative leukemia virus (MPL) is the only known definitive driver of MK differentiation from HSCs. According to the classical model of hematopoiesis, each mature MK is derived from an HSC that sequentially transitions through a multipotent progenitor (MPP), common myeloid progenitor (CMP), MK/erythroid progenitor (MEP), and MK progenitor (MKP) states. 12 In addition, MKs can also differentiate directly from repopulating HSCs that are partially or completely biased towards MKs. 13,14 After MKs are terminally differentiated, they undergo maturation, increase in size, become full of platelet-specific granules, and undergo endomitosis, nuclear replication without cell division. Endomitosis leads to polyploidy and therefore a significant increase in the amount of DNA each MK harbors, reaching up to 128N within one nuclear membrane. 15 MKs then assemble and release platelets by extending long proplatelet extensions through endothelial cells and into the bloodstream. 16 The present studies have revealed that both MK and platelet counts can be significantly increased by enhancing DNA damage.

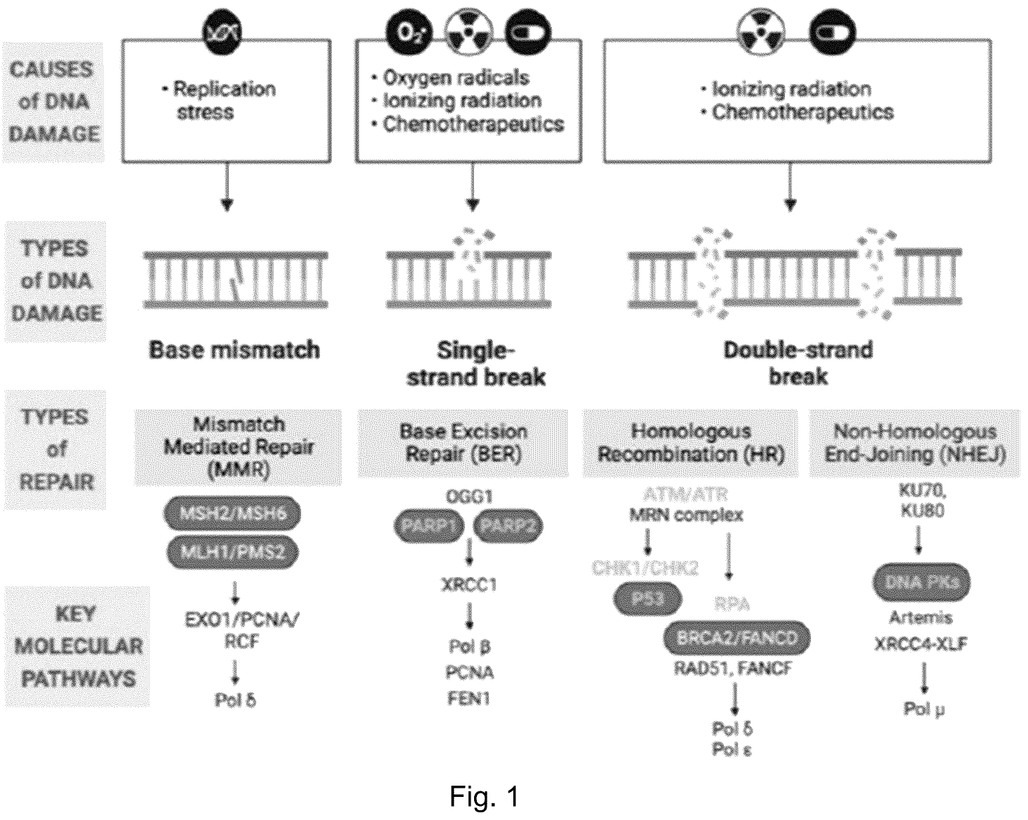

DNA lesions and the DNA damage response. DNA damage is an inevitable event in the life of a cell, caused by either normal metabolic processes or exposure to DNA damaging agents. 17 If left unrepaired, DNA lesions can cause mutations that hamper cellular function and are contributing factors to human disease, cancer, and aging. 18 Therefore, the ability of any given cell to cope with the formation of lesions in its genetic material and maintain its genomic integrity is essential for tissue development and organismal homeostasis. Genomic integrity is maintained through a variety of repair processes that respond to and coordinate cellular responses, which are summarized in FIG. 1. Collectively, these processes compose the DNA damage response (DDR). 17 Several repair pathways exist that efficiently remove specific types of DNA lesions, including Nucleotide Excision Repair (NER), Base Excision Repair (BER), Non-Homologous End-Joining (NHEJ) and Homologous Recombination (HR) (FIG. 1). The DDR is highly regulated in the hematopoietic system, and its importance is highlighted by several diseases where this response is compromised, including Fanconi anemia, Nijmegen breakage syndrome, ataxia-telangiectasia, dyskeratosis congenita, and increased cancer predisposition. 19

Modulation of DDR as a therapeutic strategy. Genetic instability is a hallmark of cancer20, with tumor cells exhibiting an increased amount of unrepaired DNA lesions. While accumulation of these lesions culminates in increased mutational burden and is associated with tumor aggressiveness, DNA damage also creates tumor-specific vulnerabilities that can be clinically exploited. Therapeutically targeting the DDR has gained momentum over the last decade, particularly for solid tumors. Inhibitors of PARP1, a sensor of DNA damage that is central to DDR, have become increasingly more common. 8 In physiological conditions, PARP1 binds to sites of DNA damage and recruits DNA repair effectors that complete repair of lesions. PARP inhibitors such as Niraparib “trap” PARP at sites of lesions, which leads to stalling of DNA replication forks and formation of toxic double-stranded breaks (DSBs) in DNA. 8 Inhibition of PARP1 by itself is not sufficiently for lethality, as its activity can be compensated by other repair pathways. However, in the absence of BRAC1/BRCA2 (key elements of HR DNA repair), the combined inhibition of PARP1 with lack of BRAC1/BRCA2 leads to accumulation of unrepaired DNA lesions, triggering cell death. 8 This synthetic lethality increases specificity and efficiency of treatment, and three of these drugs (Olaparib, Niraparib, and Rucaparib) have become widely used in the treatment of BRAC1/BRCA2 mutated ovarian and breast cancers. 8,9

Notably, PARP inhibitors have been reported to cause anemia and thrombocytopenia in patients 8,9, which suggests that increased amounts of unrepaired DNA damage would reduce platelet counts in humans. Indeed, these results showing an increase in MK and platelet counts with Niraparib treatment that were unexpected. However, therapeutic dosing of Niraparib in patients starts at 200 mg/kg, and additional experiments in mice revealed that dosing mice at higher concentrations causes both thrombocytopenia and anemia, consistent with reports in patients (see data herein). This indicates that there is a threshold of DNA damage that is tolerable, prior to induction of thrombocytopenia. Further, it reveals that there is a concentration window at which induction of moderate levels of DNA damage increase MK and platelet counts, but high levels of DNA damage lead to thrombocytopenia.

The idea that DNA damage can positively impact the differentiation and development of MKs and platelets has not been previously described and has not been previously studied, despite the unique features of MKs in regards to DNA biology (i.e. extremely high ploidy; endomitosis).

The work described herein will utilize END-seq, a high-resolution genome wide technology to precisely map the location of DNA breaks during different stages of MK maturation. 23 Utilizing this unbiased technology, it can be determined, at single nucleotide resolution over the entire genome, the location of DNA breaks during megakaryopoiesis. This data will indicate if mutations at specific regions of the genome are relevant for MK cell maturation, and how these influence genome organization, replication fork progression, and chromosomal fragility in these populations. Single-cell tracking of time-lapse imaged colony-forming unit assays will be performed. 24 These experiments will determine the fate specification of human MEPs using long-term (>7-day), single-cell, time-lapse imaging which records division rate, velocity, viability, and probability of lineage commitment at the single-cell level over time to observe and measure dynamic change. Notably, while still elusive in MK and platelet biology, the mechanisms and consequences of DNA damage accumulation have been well characterized in other blood cells and processes. 25-28 The methods described herein can meet a significant unmet clinical need to modulate thrombopoiesis and mitigate abnormal platelet counts in patients.

### Approach

### Data

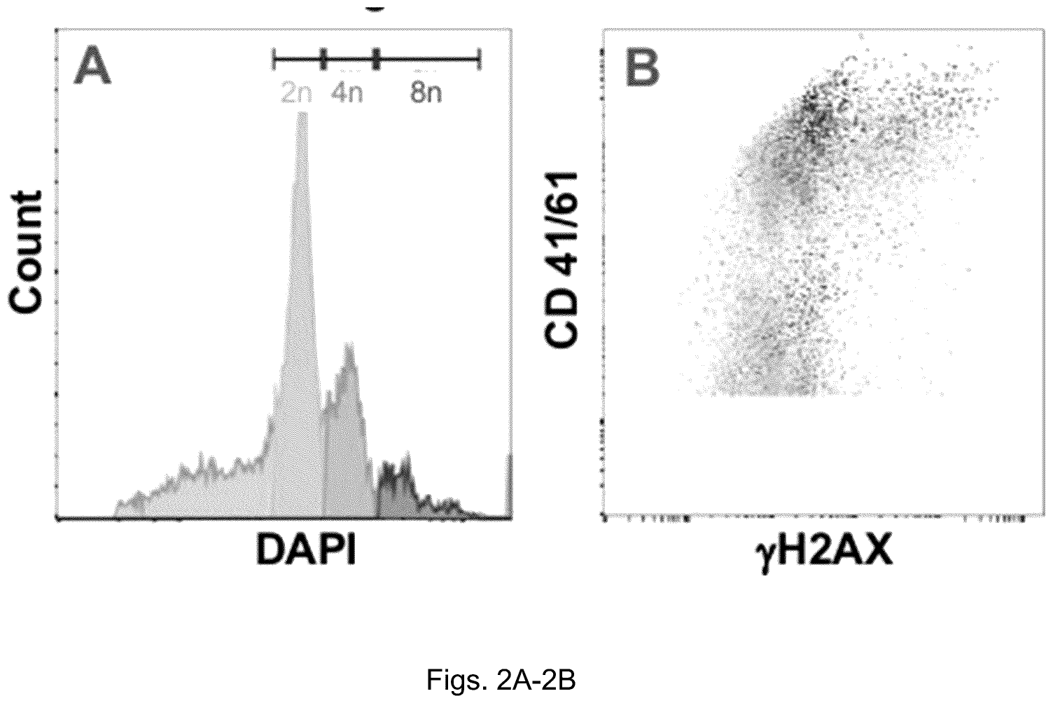

DNA damage accumulation during megakaryopoiesis. As little is known about DNA damage in MKs over their development, the amount of DNA damage in MKs was first quantified over the course of their maturation. Human CD34+ HSPCs were isolated by magnetic activated cell sorting from the mononuclear population of cells extracted from human femoral heads discarded following routine hip arthroplasty. Sorted HSPCs were differentiated into MKs by culture in STEMSPAN™ expansion media supplemented with recombinant human Stem Cell Factor (SCF) and TPO. After 10 days of culture, cells were stained for the CD41/CD61 receptor complex before intracellular staining with an anti-γH2AX antibody to assess DNA damage. Ploidy was analyzed by measuring DNA content by flow cytometry (FIG. 2A), revealing an increase in γH2AX signal with increasing ploidy (FIG. 2B). These results indicate that as MKs mature and duplicate their DNA content, they accumulate DNA damage.

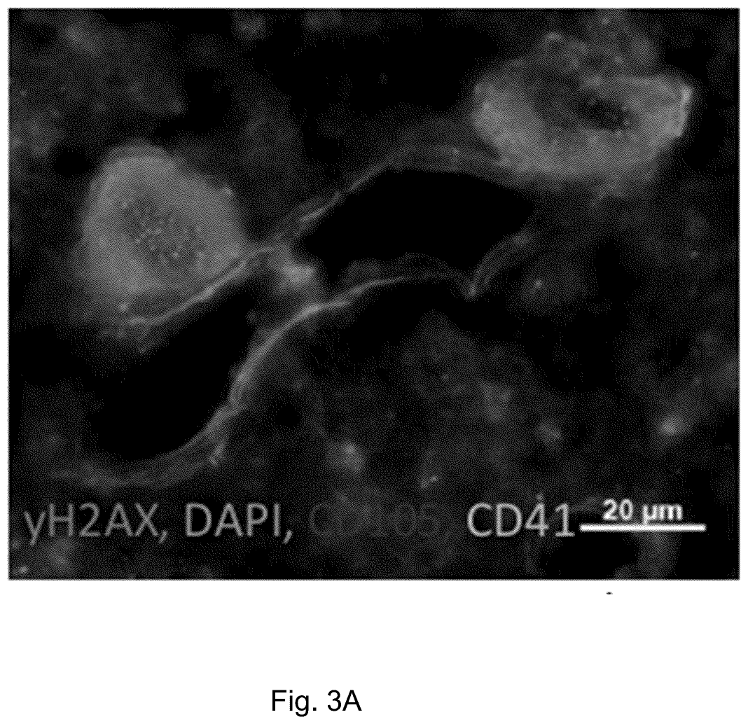

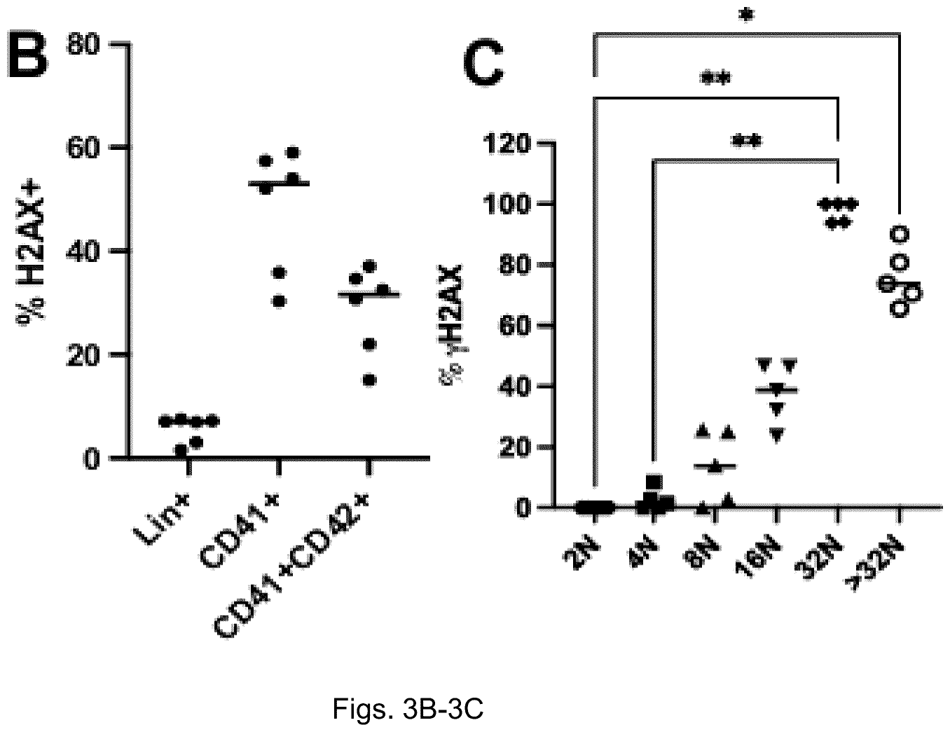

The findings where then confirmed and extended in mice. To visualize the distribution and amount of DNA damage, femoral bone marrow sections were probed for γH2AX and found that a significant proportion of mature MKs presented with pan-nuclear staining containing multiple γH2AX foci (FIG. 3A). Furthermore, γH2AX in MKs were quantified in native bone marrow. 29 MKs were labelled with CD41 and CD42d antibodies to differentiate MK progenitors and mature MKs, respectively.

Other mature linages were defined by staining with NK1.1 CD3, CD19, B220, Gr-1, and CD11b to label NK cells, T cells, B cell, granulocytes and monocytes, respectively. 29 This experiment revealed a higher prevalence of γH2AX in both immature MKs (CD41+) and mature MKs (CD41/CD42d+) compared to other differentiated bone marrow cells (FIG. 3B). In addition, a significant, positive correlation was identified between prevalence of DNA damage with ploidy, with almost 100% of 32N MKs being positive for γH2AX (FIG. 3C). Together, these data indicate that both human and murine MKs tolerate the accumulation DNA damage through their maturation, which increases with ploidy level.

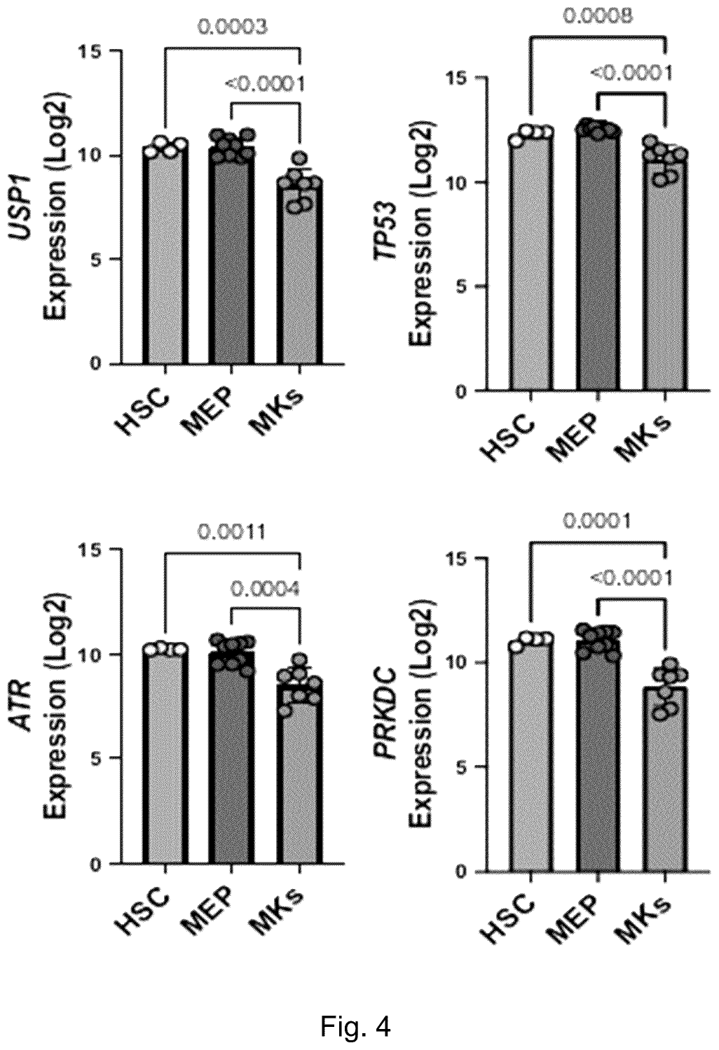

It was explored how MKs accumulate these high levels of DNA damage without undergoing cell death. DNA damage can be repaired through a variety of pathways. The normal human hematopoiesis dataset accessed via Bloodspot, from Novershtern et al 1 was utilized to probe the expression of various DNA damage repair genes in MKs (CD34−CD41+CD61+CD45−), bipotent MEPs (CD34+CD38+IL3R-CD45RA-), and CD34+ HSCs (CD38-CD34+). Notably, expression of DDR related genes such as ATR, TP53, DNAPK, and USP1 were significantly downregulated in MKs compared to their bipotent MEPs as well as HSCs (FIG. 4). Without wishing to be bound by theory, these findings indicate that MKs can downregulate the expression of DNA repair genes relative to their progenitors as a mechanism to override DDR. The type of DNA lesions that are formed will be determined as described below, along with where these lesions are located, which DNA repair pathways are activated throughout MK maturation, and how DNA damage affects cellular functions such as DNA replication during endomitosis.

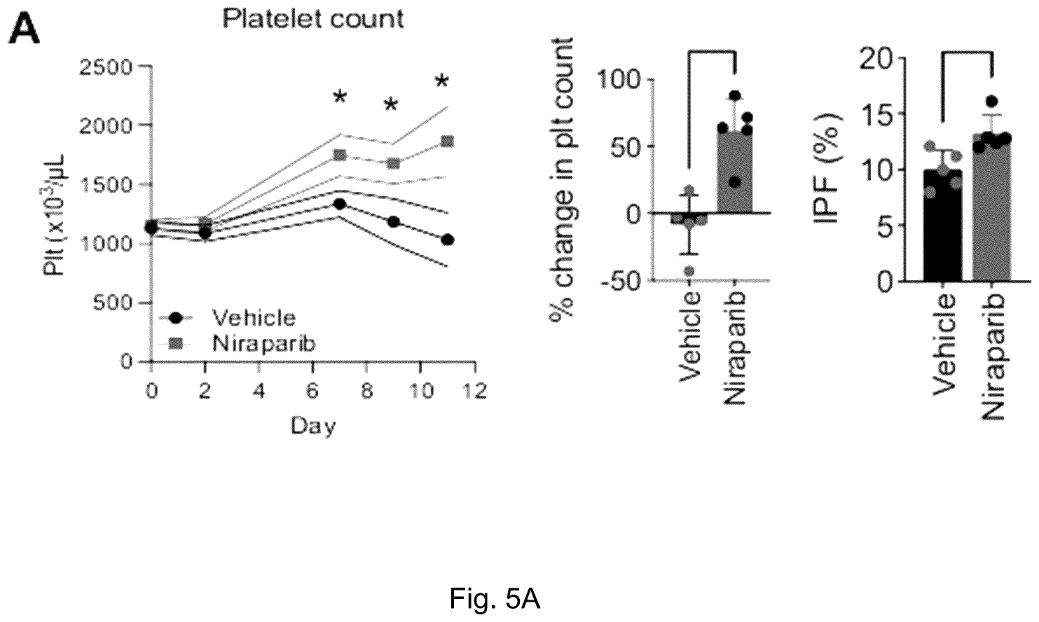

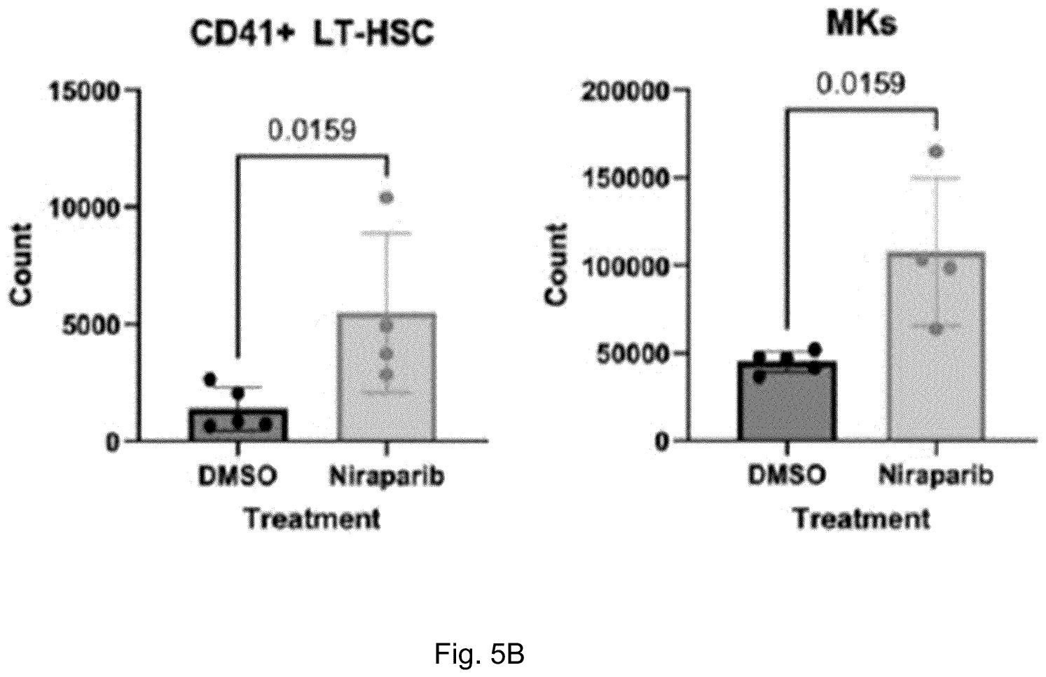

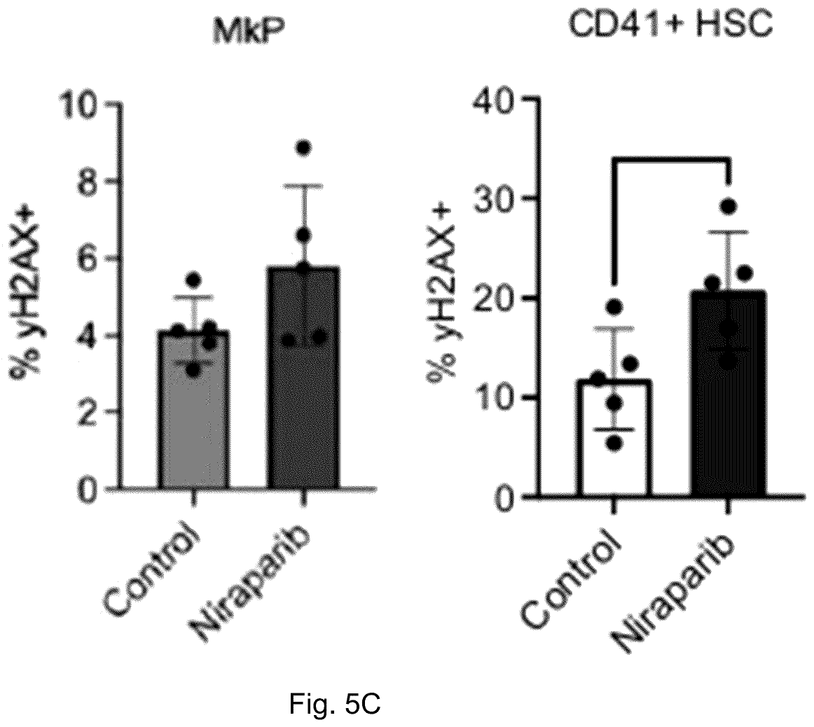

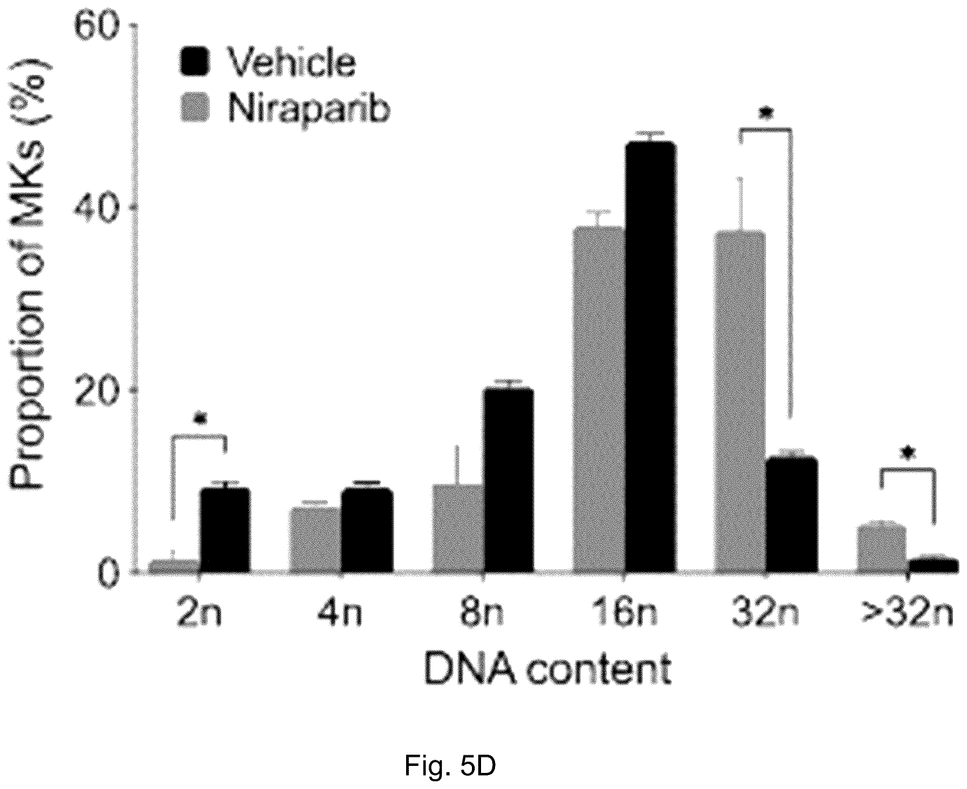

PARP inhibition increases MK differentiation. The data revealed pan-nuclear γH2AX staining (FIG. 3) and expression (FIGS. 2 and 3) in both human and murine MKs in normal physiology. As γH2AX is indicative of DSB formation, the consequences of accumulating increased amounts of unrepaired DNA damage during MK maturation were investigated. Mice were treated with a subtherapeutic dosage of the PARP inhibitor Niraparib (25 mg/kg, intraperitoneal) or saline control for 11 days. The dosing schedule was as follows: 5 consecutive days of Niraparib treatment followed by 2 days of drug holiday (no injection), then 4 more consecutive days of Niraparib treatment. Mice were sacrificed on day 11. Platelet counts were taken on days 0 (pre-treatment), 2, 7, 9, and 11. Cohorts of mice were sacrificed on days 3 and 11 for quantification of bone MK and progenitor populations. Notably, from the day 7 time point, there was a significant increase in the platelet counts and immature platelet fraction in the Niraparib-treated mice (FIG. 5A) compared to the saline control. This can be explained by a significant increase in both MKs and the MK-committed HSCs (CD41+LT-HSCs) on the days preceding the increase in platelet counts (FIG. 5B), suggesting that PARP inhibition led to an increase in MKs through both traditional and direct differentiation from HSCs. To. To directly tie increased MK and platelet counts to DNA damage induction, the proportion of MK progenitor cells positive for γH2AX after

Niraparib treatment were measured. Notably, the number of MK-committed HSCs positive for γH2AX was significantly increased while there was a trend to more γH2AX-positive MKPs (FIG. 5C), substantiating the effect of Niraparib on DNA and connecting DNA damage to the platelet phenotype. MKs in mice treated with Niraparib for 11 days also had a striking increase in ploidy; there were almost no 2N MKs while there was a large increase in the number of 32N MKs (FIG. 5D). These data reveal that despite PARP inhibition, MKs continued to mature and replicate DNA, even with the accumulation of DNA breaks.

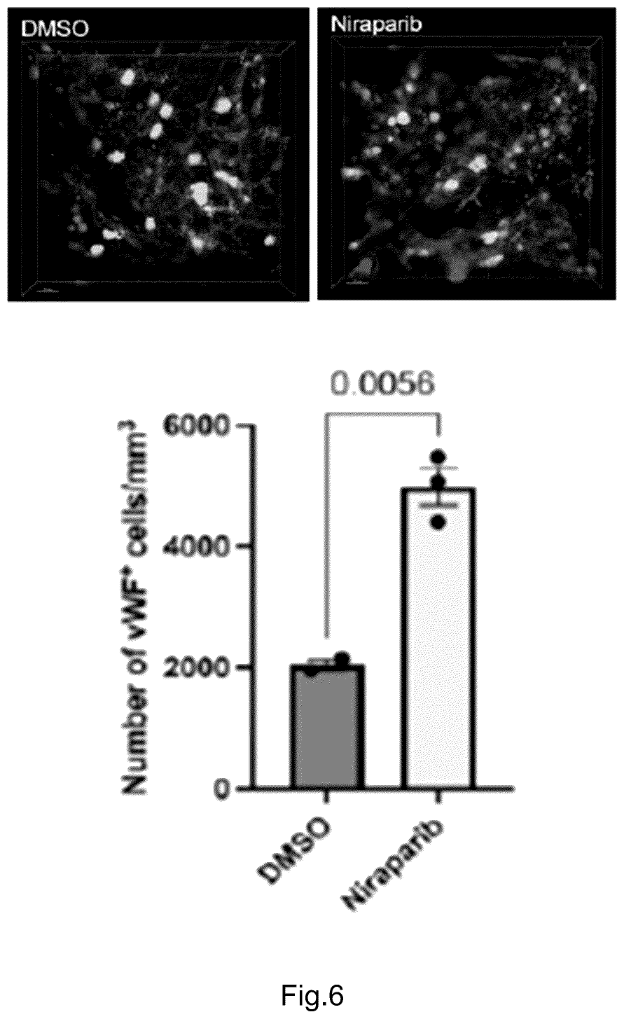

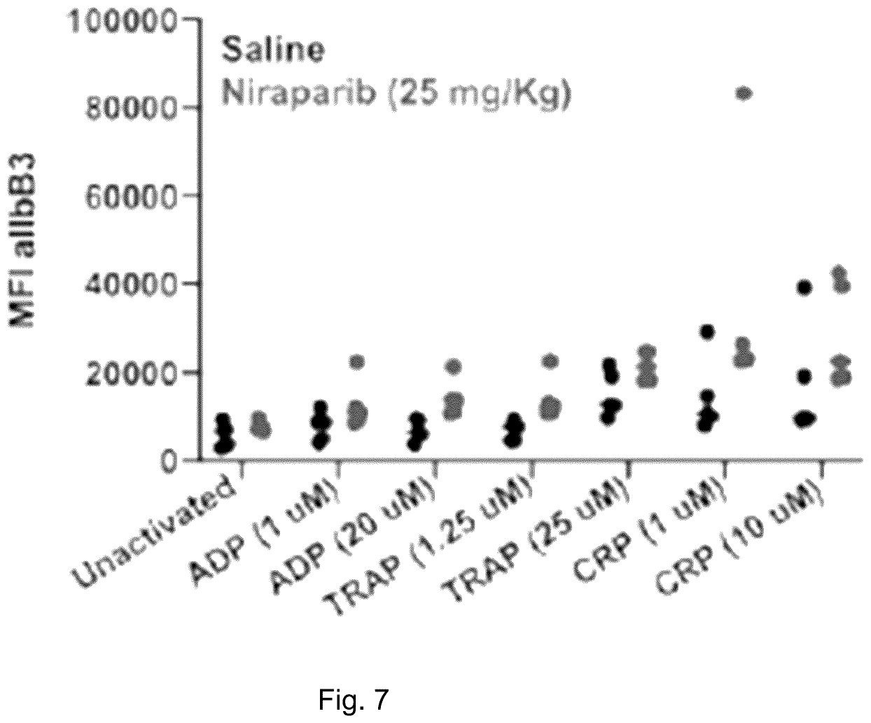

While MK ploidy is not directly causative of platelet production, higher ploidy MKs are associated with increased platelet production and ploidy status is indicative of more terminal MK maturation. To look further at live MKs, 2-photon intravital microscopy (2PIVM) was performed in the calvaria of vWF-GFP reporter mice (MKs and platelets are GFP+) treated with 25 mg/kg Niraparib or vehicle control DMSO for 7 days. Mice were anesthetized and a window in the calvaria was made to view the exposed bone marrow. MK distribution and proplatelet formation was observed for 5 minutes in 4 different fields of view per mouse. MK quantification by this method corroborated the increased MK number in the bone marrow in Niraparib treated mice (FIG. 6). Critically, platelet activation was also measured after Niraparib treatment to determine if the increased platelets produced are functionally altered. Indeed, FIG. 7 reveals no changes in activation in platelets stimulated with ADP, TRAP or CRP after in vivo Niraparib treatment.

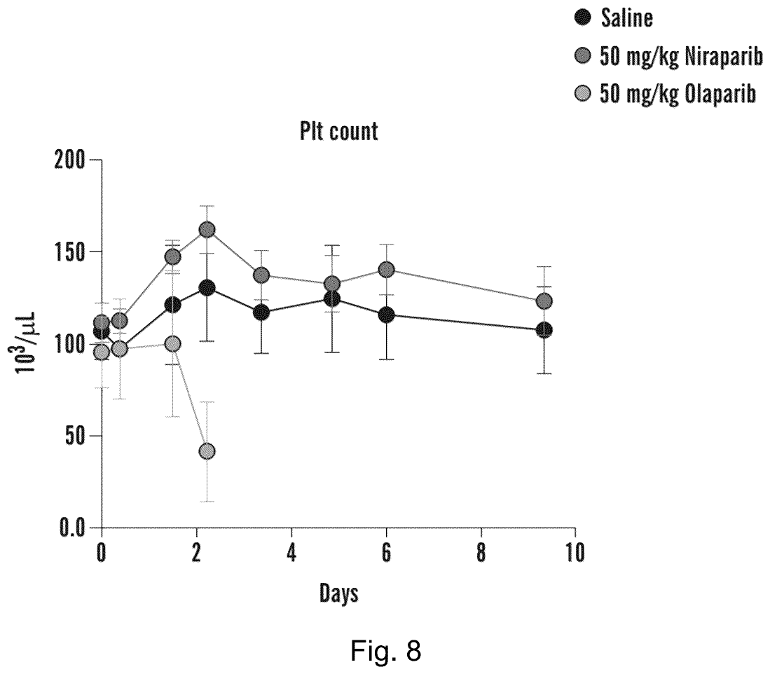

Next, the inventors substantiated that PARP inhibitors can lead to thrombocytopenia at higher dosages. 8,9 Mice were treated with 50 mg/kg of Niraparib and Olaparib for 10 days. After 3 days of Olaparib treatment, however, mice presented with severe thrombocytopenia that resulted in early termination of the Olaparib experiment (FIG. 8). Niraparib treatment had a trend to increased platelet counts but did not have the significant effect it did at 25 mg/kg (FIG. 5A, FIG. 8). Together these data illustrate the threshold effect of DNA damage on MK and platelet production and that this effect can be induced in vivo.

The impact of increasing amounts of DNA damage on different HSPC populations and its functional outcome on MK cell viability, output, and ultimately platelet production will be determined. It will also be examined whether the increase in MK and platelet counts are TPO-independent. Both male and female adult mice (8-12 weeks) and the studies will be powered to analyze all readouts as a function of sex. CD34+ peripheral blood cells will be obtained from both male and female donors, and experiments done with cells derived from ‘male’-versus ‘female’-derived cells documented. If analyses indicate that there are no statistical differences between outcomes, data will be combined. If the readouts are different, these important sex-specific differences we be reported and analyzed separately.

It is contemplated herein that MKs have evolved unique mechanisms to tolerate the accumulation of DNA damage, continue endomitosis, and produce platelets. The data presented herein indicate that accumulation of DNA damage is a driver of MK maturation and lineage specification. These hypotheses will be tested and the type of DNA lesions that occur during MK maturation, the role of replication of damaged DNA during MK maturation, the locations of these lesions in the genome, and the DNA response and repair pathways activated during the MK maturation process will be determined.

### Determining the Type of DNA Damage Lesions Accumulated Throughout MK Maturation

The instant data revealed high levels and pan-nuclear staining of γH2AX, a classic marker for DNA damage, in both human and murine bone marrow MKs that increased with differentiation and ploidy (FIGS. 2, 3). It will be determined which type of DNA lesions accumulate and if the spectrum of lesions changes as MKs mature. While γH2AX is a marker for DSBs, these could be generated directly or indirectly, from incorrect processing of single strand breaks that accumulate during MK maturation. The mechanisms leading to γH2AX accumulation during MK maturation will be identified, as well as, at different ploidies, which type of break is prevalent.

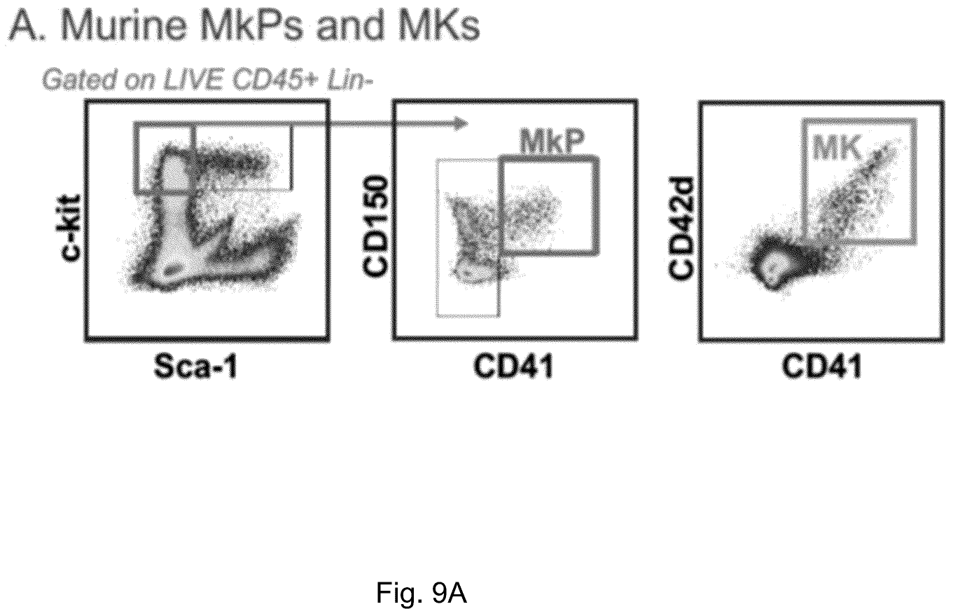

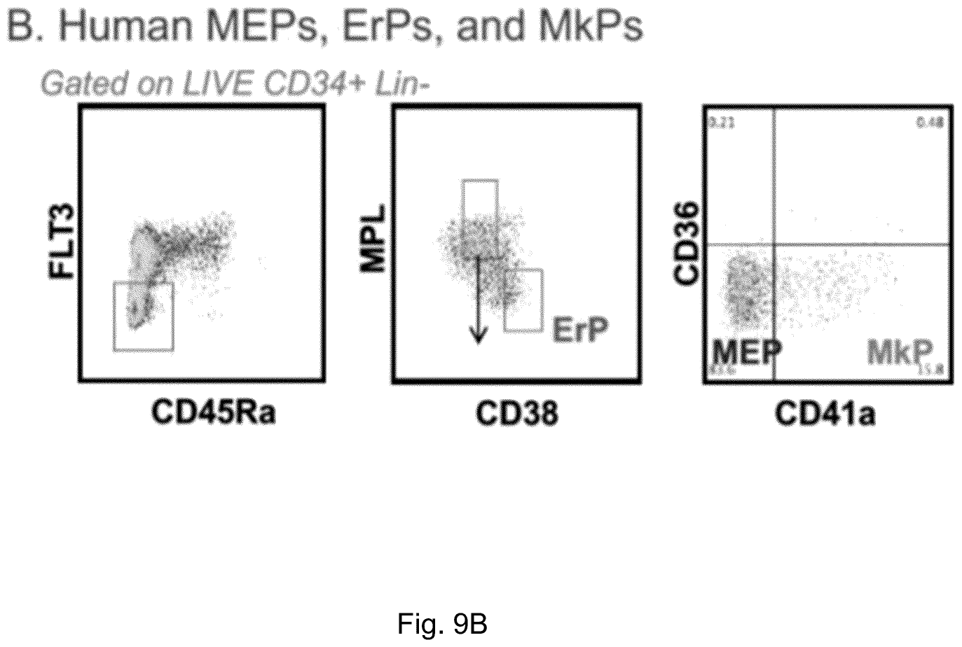

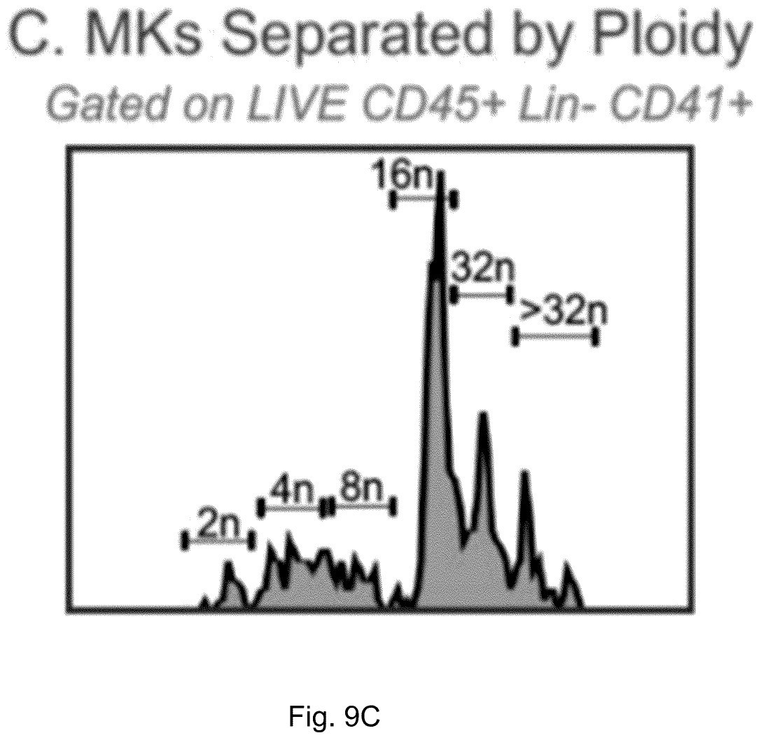

For all experiments, MKs will be generated from either 1) primary murine HSPCs from bone marrow isolated from the long bones and iliac crest of mice or 2) single-donor primary human CD34+ peripheral blood cells (purchased from Stem Cell Technologies). For murine HSPCs, lineage positive cells will be removed from total bone marrow suspension by antibody depletion as previously described. 29 For both murine and human cultures, cells will be cultured with TPO (70 ng/ml) for 4 and 10 days, respectively. 29 Populations of MKs and their progenitors will then be distinguished and/or isolated by flow cytometry using the gating schematics outlined in FIG. 9. 29-31 This also includes identification and sorting of mature human and murine MKs by ploidy level, as done in FIGS. 2 and 3, respectively. To note, for ploidy analyses, murine MKs can be sorted into 2N, 4N, 8N, 16N, 32N, and >32N populations, as seen in FIG. 9C and FIG. 3, while human MKs can be sorted into 2N, 4N, 8N, and >8N populations (FIG. 2).

These experiments will use both murine and human MKs. To discern the relative contribution of each type of DNA lesion (single-versus double-stranded) at each stage of MK maturation/ploidy, single cell gel electrophoresis (Comet assays) 32 will be performed to determine which type of break is prevalent. While alkaline Comet assays (performed at pH 8) detect both types of lesions, neutral Comet assays are specific for double-stranded breaks. Etoposide and gemcitabine treatments of 2N MKs (20 and 2 μM, 24 hours, respectively) will be used as a positive control as these drugs induce double-stranded and single-stranded DNA breaks, respectively. Untreated CD34+ HSCs will be used as a negative control. By performing both assays it will be possible to both discern the relative contribution and quantify the amount of each type of lesion.

One of the most common causes of DNA damage and subsequent lesion accumulation in mammalian cells is stalling of RNA polymerase II (RNA pol II), which triggers substantial genetic instability. 33 Therefore, it will be determined if accumulation of DNA lesions during MK maturation is potentiated by blocked transcription complexes. RNA pol II will be inhibited with the drug 5,6-dichloro-1-beta-D-ribofuranosylbenzimidazole (DRB, dose curve of 1-100 μM) or vehicle control added to the culture media on days 3 (murine) and 9 (human) for 24 hours, immediately preceding polyploidization as to not affect MK differentiation. Etoposide treatment of 2N MKs (20 μM for 24 hours) will be used as a positive control. There will then be performed quantification of 1) phosphorylation of γH2AX by flow cytometry as in FIGS. 5 and 2) the type and quantity of DNA breaks by comet assays. If a reduction in either of these levels is observed, this will indicate that DNA damage in MKs consists mostly of “bulky” lesions able to block transcription complexes, which then trigger the formation of DNA breaks. Combined, these experiments will determine the spectrum and quantity of DNA lesions that arise during MK maturation, if they are generated through RNA pol II stalling, and how they relate to increasing ploidy during this process.

Understanding the dynamics of damaged DNA replication during MK maturation.

A key part of MK maturation is endomitosis, a process where DNA replication happens without cell division. 16 This leads to the formation of cells with multiple copies of their genome (up to 128N), packed into a single nuclear membrane. Under physiological conditions, Replication Protein A (RPA) participates in both the initiation and elongation steps of DNA replication by enhancing the assembly and recruitment of DNA polymerases a, 8, and & (see Schematic, FIG. 1). 34 Problems in DNA replication (i.e. DNA lesions) are signaled via the detection of persistent stretches of RPA-ssDNA by the ATR kinase. RPA is phosphorylated at sites of ssDNA lesions and signals activation of different DNA repair pathways, including NER, BER, and HR. 35 As MKs have increased amounts of DNA, and lower expression of DNA repair genes (FIG. 4), it is hypothesized that MKs will show increasing phosphorylation of RPA and that replication fork dynamics will be altered with shorter replication traces caused by increased amounts of replication hampering lesions. This response will be analyzed to decipher the link between the repeated cycles of DNA replication during endomitosis with DNA damage accumulation and understand the relevance of this process to MK maturation. It will also determined if and how accumulation of DNA lesions during replication affects MK cell biology and viability.

These experiments will be performed with MKs generated from single-donor primary human CD34+ peripheral blood cells. On Day 10, MKs will be isolated and both ploidy and phosphorylation of RPA28 will be measured using flow cytometry (FIG. 9C, 2N, 4N, 8N, >8N). Etoposide-treated MKs will be used as a positive control. Increases in RPA phosphorylation will indicate that despite the continuous cycles of endomitosis, MKs accumulate DNA lesions that slow progression of replication forks.

As DNA replication through endomitosis is important for MK maturation, the progression of replication forks during this process will be analyzed through DNA fiber assays. This assay will permit study of alterations in replication fork dynamics at a genome-wide level, but at a single-molecule resolution. DNA fiber assays rely on the incorporation of nucleotide analogs into nascent DNA during replication, which is then measured to monitor different replication parameters, including fork progression, restart, and termination. 36 Crucially, this assay can also be utilized to detect the formation of ssDNA gaps that are commonly formed during replication stress, by treatment of post-replicative samples with S1 nuclease, which will specifically cleave ssDNA gaps. 36 In short, day 10 sorted mature MK populations at different ploidy levels will be subject to a dual-labelling system (two consecutive nucleotide analogs), that will allow quantification of replication fork progression. Cells will initially be labelled for 30 min with CldU (5-chloro-2′deoxyuridine; red), followed by 30 min labeling with IdU (5-iodo-2′deoxyuridine; green). Cells will be washed, collected, and lysed. Nascent DNA will then be spread on angled-slides, and fixed following standard protocols. 37 Analysis of fibers will be performed by fluorescence microscopy and DNA fiber lengths will be quantified. The ratio of red/green incorporation will indicate the dynamics of DNA replication forks in MKs as these cells mature. Experiments will also be performed where S1 nuclease is added prior to DNA fixation, which will indicate, at each ploidy level, the extent to which breaks in DNA are hampering replication fork progression. As a control for these assays, cells will be treated with hydroxyurea following the first labeling (CldU), prior to the second labeling (EdU). Hydroxyurea causes replication fork stalling, providing a positive control for the dynamics of replication in perturbed settings.

These experiments will detail replication dynamics and how it relates to DNA damage accumulation during MK maturation.

Determining the location of DNA lesions during megakaryopoiesis.

The present data demonstrate that DNA damage accumulates as MKs mature, in direct correlation with ploidy stage (FIGS. 2, 3). The location of DNA damage during this process could lead to activation of different repair pathways and stress responses. Additionally, damage in specific areas of the genome could play a role in cell fate or function. Notably, it has recently been shown that telomere

attrition skews HSC differentiation towards the megakaryocytic lineage. This is a unique response to telomere damage in the hematopoietic system, where telomere instability is traditionally associated with impaired hematopoietic potential due to activation of DDR pathways38 and reduced hematopoietic yield in patients. 39 Therefore, it is hypothesized that accumulation of DNA damage in MKs is preferentially localized to telomeric sites. It will be determined if DNA damage is occurring at telomeres and END-seq used to map where DSBs are occurring across the MK genome.

These experiments will be done with MKs generated from CD34+ cells sorted and isolated by ploidy, as described above. The specific accumulation of DNA damage at telomeric sites in MKs at each ploidy level will be probed by performing telomere dysfunction induced foci (TIF) analysis in these cells. In brief, cells will be collected, cytospun onto glass slides, permeabilized, and stained with antibodies against 53BP1 (indicative of DNA damage) and peptide nucleic acid (PNA) probes specific against telomeric DNA. The number of colocalized foci will be quantified in each MK population, representing sites of DNA damage specifically at telomeres. A genetic approach will also be taken by performing experiments where damage is induced specifically at telomeric DNA and how it affects MK differentiation determined. Telomeres are usually protected by the shelterin complex, composed of TRF1, TRF2, RAP1, TIN2, TPP1, and POT140, and different groups have shown that TIFs can be induced by disruption of TRF2 and POT1.39 POT1 will be deleted (by CRISPR/Cas9) in CD34+ cells using a protocol based on the method of Montenont et al. 41, (see schematic, FIG. 10). Ablation of POT1 will be confirmed and quantified by targeted sequencing and determine increased telomeric damage by TIF assays. It will then be determined if POT1 deletion increases the number of CD34+ cells that differentiate into MKs (CD41+ cells) using flow cytometry. If telomere damage modulates MK differentiation, it is expected that deletion of POT1 will increase differentiation of MKs (increased number of CD41+ cells differentiation from CD34+ progenitor cells).

In addition to telomeres, the location of DNA lesions throughout the genome during MK cell maturation will be mapped using END-seq, a next-generation sequencing technique that allows quantitative mapping of DSBs at nucleotide resolution, across the genome. These assays will determine if DNA lesions occur at random sites, or are located to specific, predicable loci. In brief, human MKs at different ploidies will be sorted and embedded in an agarose plug (to prevent artificial generation of DNA breaks from mechanical processing and fixation). Samples will be treated with proteinase K and RNase A to digest proteins and RNA. After blunting the single-strand DNA overhangs at DSBs with exonucleases VII and T, a biotinylated hairpin adapter (“End-Seq Adapter 1”; Illumina p5 sequence) will be ligated on 3′ends. DNA will be sheared by sonication and captured utilizing streptavidin-coated beads, which will then be A-tailed and ligated with a second adapter (“End-Seq Adapter 2”; Illumina p7 sequence). Hairpins will then be removed by digestion, and DNA amplified utilizing Illumina's TruSeq barcoded primers. These libraries will then be sequenced; each first base (read #1) will correspond to the first base following a blunted DNA break. These experiments will provide a detailed description of DNA damage location during MK maturation, both at telomeres and/or throughout the genome.

### Determining Activation of DNA Damage Responses and DNA Repair Pathways During Megakaryopoiesis

The amount of DNA damage that accumulates during MK maturation indicates that DNA repair pathways are either insufficient to cope with the high DNA content these cells possess or are specifically downregulated. Notably, in silico analysis of human hematopoiesis indicates that several genes involved in different repair pathways are lower in MKs compared to HSCs and bipotent MEPs (FIG. 4). Without wishing to be bound by theory, this suggests a unique hypothesis; MKs disregard DNA repair at the expense of DNA lesion accumulation during maturation. Further, MKs may actively downregulate genes involved in DNA repair. To test this hypothesis, activation of DDR pathways will be compared in mature MKs vs. their bipotent progenitors (MEPs) upon induction of DNA damage. These experiments will directly test the hypothesis that despite accumulating DNA lesions, DDR is not activated in MKs during their maturation.

These experiments will use primary human CD34+ cells. Murine HSPCs will not be used, as the ability to isolate a defined MEP population in murine bone marrow has not been phenotypically and functionally optimized. MKPs and MKs will be isolated by flow cytometry and MKs will be further sorted into ploidy states. On day 9 of maturation, DNA DSBs will be induced in isolated populations (MEP, MKP, MK at each ploidy level) with etoposide treatment (2 and 20 μM for 24 hours).

DNA damage will be confirmed/measured in each population by quantifying γH2AX levels using flow cytometry. Cells will then be lysed and the expression of the DDR proteins measured, including ATR, ATM, CHK1, CHK2, p53 and p21 by western blot. Despite accumulation of DNA damage as MKs mature, MKs are expected to show a concomitant reduction in DDR activation compared to progenitor cells. Samples treated with etoposide will further consolidate this hypothesis, and show that as cells mature, DNA damage becomes more “tolerable” and fewer DNA damage response proteins will be activated in response to etoposide in mature MKs compared to their progenitors. These experiments will provide a detailed view of DDR responses in bipotent progenitors and MKs through MK maturation, elucidate which DDR proteins are used, and quantify the prevalence of these response pathways to DNA damage.

Anticipated Results: The current data indicate that DNA damage accumulates with ploidy and maturation status of MKs, without triggering cell death, as MKs continue to perform thrombopoiesis, which is not an apoptotic process. 42,43 Without wishing to be bound by theory, the inventors contemplate that the experiments described herein will demonstrate:

The relative contribution of each type of DNA lesion (single-versus double-stranded) at each stage of MK maturation and the role of transcription stress during that process that replication of DNA during endomitotic cycles potentiates formation of DNA lesions and alters replication forks dynamics the specific role of telomeric damage to MK cell maturation and map, at the genome level, the location of DNA lesions in MKs that repair of DNA lesions is not essential for MK maturation, unlike what is observed in most other hematopoietic lineages. It is possible that DRB treatment leads to cellular toxicity. This will not be a negative result, but in fact highly informative, as it will indicate that while DNA lesions are not toxic for MK maturation, transcription blockage is. Instead of determining the replication dynamics of MKs as they mature, utilizing DNA fiber assays, DNA replication dynamics can be studied by incorporation of radiolabeled nucleotides in cells with different ploidies. Instead of END-seq, complementary techniques to analyze DNA damage location, such as Breaks Labeling In Situ and Sequencing (BLISS) can be utilized. 44

As alternative or complementary experiment, the DDR response can be disrupted in MKs with specific inhibitors of different modulators of DDR including AZD7648, AZD0156, and AZD6738, which inhibit DNA-PK, ATM, and ATR respectively. These experiments will determine the role of DDR in the accumulation of DNA damage. These experiments will offer a detailed analysis on the mechanisms and consequences of DNA damage accumulation during megakaryopoiesis.

### Determining the Consequence of Enhanced DNA Damage on Megakaryopoiesis In Vitro and In Vivo

Without wishing to be bound by theory, the inventors hypothesize that induction of DNA damage in vivo increases MK number and subsequent platelet production by enhancing commitment of MKs at the MEP stage of hematopoiesis. There is evidence from that induction of DNA damage can play a positive role in megakaryopoiesis. The instant data revealed an increase in both platelets and MKs after treatment with 25 mg/kg of Niraparib (FIGS. 5, 6). Colony assays will be used to determine at what stage of hematopoiesis enhanced DNA damage increases MK production. It will then be determined if DNA damage-induced MK production is due to an arrest in the MEP cell cycle and mice lacking TPO and MPL used to determine if changes induced by DNA damage are independent of the TPO/MPL signaling pathway.

### Determining if Enhanced DNA Damage Increases Human MK Fate Specification at the MEP Stage of Hematopoiesis

The instant data indicate that accumulation of DNA damage increased the number of MKs that differentiated both in vitro and in vivo (FIGS. 5, 6, 7). However, accumulation of excessive DNA damage in hematopoietic progenitors that self-renew could be detrimental. Therefore, the inventors hypothesize that once DNA damage accumulates, terminal differentiation into an MK, a cell that seldom divides, but rather terminally differentiates and then fragments into platelets, may provide an “off ramp”. Specifically, it is hypothesized that increased DNA damage increases MK commitment at the MEP stage of hematopoiesis. DNA damage will be introduced to CD34+ HSCs and MEPs and determine if it biases these cells to differentiate into MKs verses erythroid cells.

These experiments will be done using primary human CD34+ peripheral blood cells. MEPs will be isolated by flow cytometry based on the protocol developed by Senada to isolate functionally accurate human progenitor populations (outlined in FIG. 9B). 30 Both CD34+ HSCs and MEPs will be treated with multiple compounds to induce different kinds of DNA damage seen in hematopoietic cells, outlined in Table 1, or vehicle control. The cells will also be treated with Niraparib to correlate results with the other experiments and data. Single-cell tracking of time-lapse imaged colony-forming unit assays will determine self-renewal and lineage specification (MK versus erythroid) in the treated HSCs and MEPs. 24 Briefly, this assay determines fate specification using long-term (>7-day) single-cell time-lapse imaging which records division rate, velocity, viability, and probability of lineage commitment at the single-cell level over time to observe and measure dynamic change. Notably, this system has been used for elucidating how exogenous factors influence the MEP fate decision. 24 For each experiment, the cell death rate, expansion capacity, division outcomes by generation (self-renewal vs. MK vs. erythroid), and lifetime will be quantified. If enhanced DNA damage increases MK commitment at the MEP stage of hematopoiesis, it is expected that treatment of both the HSCs and MEPs will skew fate specification to the MK lineage, but direct treatment of the MEP will have the greatest effect. However, if DNA damage accumulation in a less committed progenitor such as an MPP is essential for MK fate determination, treatment of the MEP may have little effect on MK lineage commitment. For example, in this assay, treatment of MEPs with TPO and EPO do not instruct MEP lineage commitment to MK vs erythroid, as TPO and EPO must be administered to early progenitors to be effective. 24 These experiments will determine if DNA damage influences MK fate specification at the MEP stage of hematopoiesis or in an earlier progenitor, the dynamics of this process, and what type of DNA damage is most efficient at driving this process.

Determining if the Mechanism by which DNA Damage Enhances MK Count is Via Biased Fate Specification Through Increased Cell Cycle Length in MEPs

Cell cycle dynamics revealed that the speed/rate of the cell cycle at G1 for the bipotent MEP impacts cell fate. 11 Specifically, cells with a slower cell cycle are more likely to differentiate into MKs, while cells that cycle faster will differentiate into RBCs. 11 Notably, DNA damage stalls differentiation at G1/S, thus extending the cell cycle. The inventors hypothesize that the mechanism by which DNA damage accumulation biases fate specification to the MK lineage is through increasing cell cycle length. This will be tested by inducing DNA damage in MEPs and analyzing their cell cycle profile. This will be directly correlated with the data which measures multiple parameters of fate specification of MEPs treated with the same DNA damage inducing compounds.

These experiments will use primary MEPs derived from human CD34+ peripheral blood cells. MEPs will be treated with the compounds outlined in Table 1 to investigate if changes in cell cycle is the mechanism by which DNA damage biases fate determination. During MEP treatment, cells will be labeled with carboxyfluorescein diacetate succinimidyl ester (CFSE) to track cell divisions (2 μM for 10 minutes at 37° C.) following the protocol of Lu et al. 31 After 72 h of culture with CFSE, cells will be labeled with anti-CD41 APC-H7 and anti-CD36 PE to detect MKs and erythrocytes, respectively, and analyzed for CSFE intensity using flow cytometry. The CFSE intensity versus cell surface expression of CD41 and CD36 representing MK or erythroid lineage specification, respectively will be assessed. Specifically, gating on the fastest-cycling (lowest CFSE intensity) cells will reveal the most erythroid-specified cells (CD36+CD41−), whereas MKs (CD36−CD41+) will be enriched in the slowest-cycling (highest CFSE intensity) population. For each compound administered to MEPs (Table 1), the shift in CFSE intensity and its associated cell type (MK vs. erythroid) will be quantified, which will denote how that treatment impacts both cell cycle speed and cell output.

To validate the inferred fate of these cells colony assays will be performed. The three CFSE groups will be sorted and fate specification confirmed by performing dual MK and erythroid CFU assays using a protocol adapted from Sanada et al. 30 Briefly, equal numbers of cells will be seeded in MegaCult medium (with added rhEPO, rhIL-3, rhIL-6, rhSCF, and rhTPO) for 48 h. MKs will then be stained using an anti-CD41 antibody and colonies will be scored as BFU-E (erythroid), CFU-MK/E (bi-potent ability), or CFU-MK (MK), with MK+ colonies being identified based on CD41 positivity. MEPs will be treated with rapamycin (50 nM) as a positive control, as it robustly slows cell cycle speed and increases MK-CFU formation. 31

Together, these experiments will determine how inducing various forms of DNA damage affect cell cycle speed and MEP lineage determination. Thus, they will reveal if the mechanism by which accumulation of DNA damage leads to MK fate determination at the MEP stage is through increased cell cycle length.

### Determining if the DNA Damage-Induced Increase in MKs and Platelets In Vivo is Dependent on Signaling Through the TPO/MPL Pathway

TPO is the only known definitive driver of MK fate specification from HSCs through classical hematopoiesis. However, as it is hypothesized that DNA damage accumulation increases MK differentiation later in hematopoiesis, this suggests that the impact of DNA damage on fate specification may not be TPO-dependent. Notably, the instant data show that treatment of mice with Niraparib lead to a marked increase in platelet counts (FIGS. 5, 6). As such, targeting DNA damage or repair or repurposing PARP inhibitors at subtherapeutic dosages is a viable option for treating patients with thrombocytopenia. Determining if the mechanism of enhanced MK number is TPO dependent may guide selection of patients who will respond particularly well to these therapies. In vivo Niraparib treatment of wildtype mice to mice deficient in TPO or the TPO receptor, MPL. This will determine if the increase in MKs and platelets with Niraparib treatment is dependent on the TPO/MPL pathway.

These experiments will use wildtype C57B16 mice, mice deficient in TPO (Tpo−/−), and mice deficient in the TPO receptor, MPL (cMpl−/−). Niraparib will be administered to mice as outlined in FIG. 5. Complete blood counts will be taken using 5 μL of whole blood on days 0 (pre-treatment), 2, 4, 7, 9, and 11. Upon sacrifice, bone marrow HSPC (LT-HSC, ST-HSC, MPP2, MPP3, MPP4, MKP) and MK populations will be quantified in conjunction with γH2AX signal by flow cytometry, as done in FIG. 5B, C. MK ploidy will also be quantified by flow cytometry. The sternum of each mouse will be isolated and embedded for sectioning and staining to visualize and quantify MKs (CD41+) and DNA damage (γH2AX+ foci). If the mechanism by which DNA damage accumulation increases MK and/or platelet counts is dependent on signaling through TPO or the MPL pathway, then there will be no increase in MKs or platelets in Tpo−/− or cMpl−/− mice, respectively. Together, the these experiments will determine the dependence of DNA damage accumulation on both TPO and its receptor, MPL.

Anticipated Results: These experimented will determine if DNA damage increases MK number by skewing MK fate specification at the MEP stage of hematopoiesis. It will also measure the dynamics of this process and determine what type of DNA damage is most efficient at driving this process. The mechanism of how MEP lineage determination is being altered will be probed by inducing various forms of DNA damage and measuring how it affects cell cycle speed and subsequent

MK versus erythroid cell differentiation. Finally, the therapeutic utility of DNA damage inhibitors will be further demonstrated, e.g., Niraparib for the treatment of patients with thrombocytopenia. Genetic murine models will be used to determine if the increase in both MKs and platelets seen with Niraparib treatment is dependent on signaling through the TPO/MPL pathway.

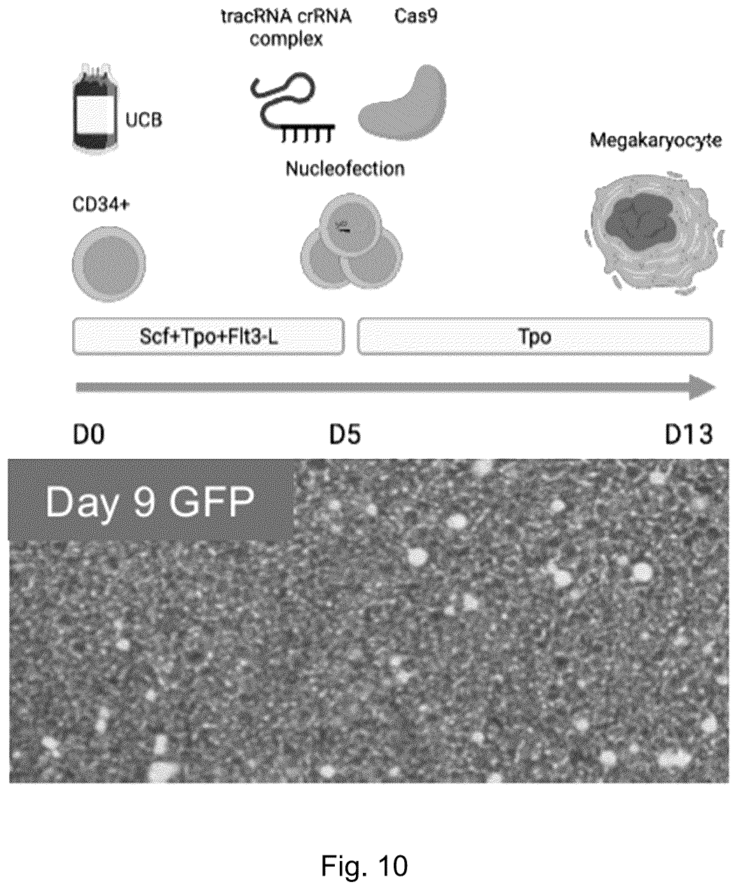

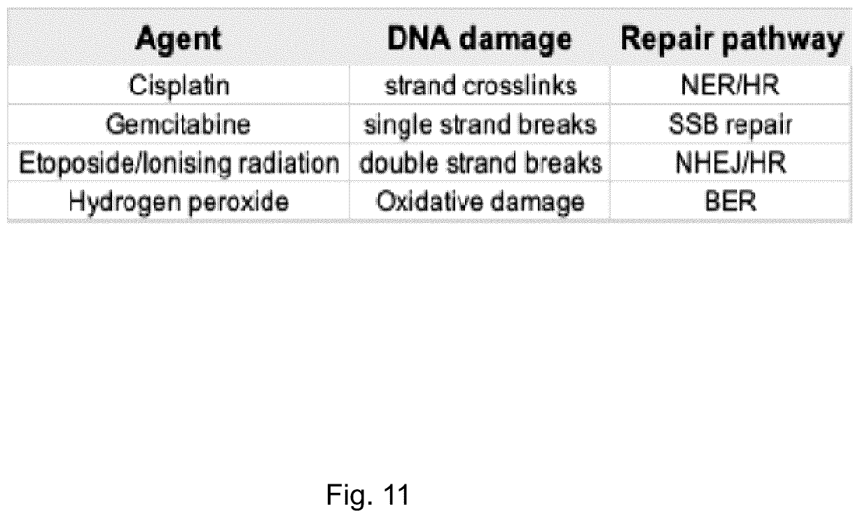

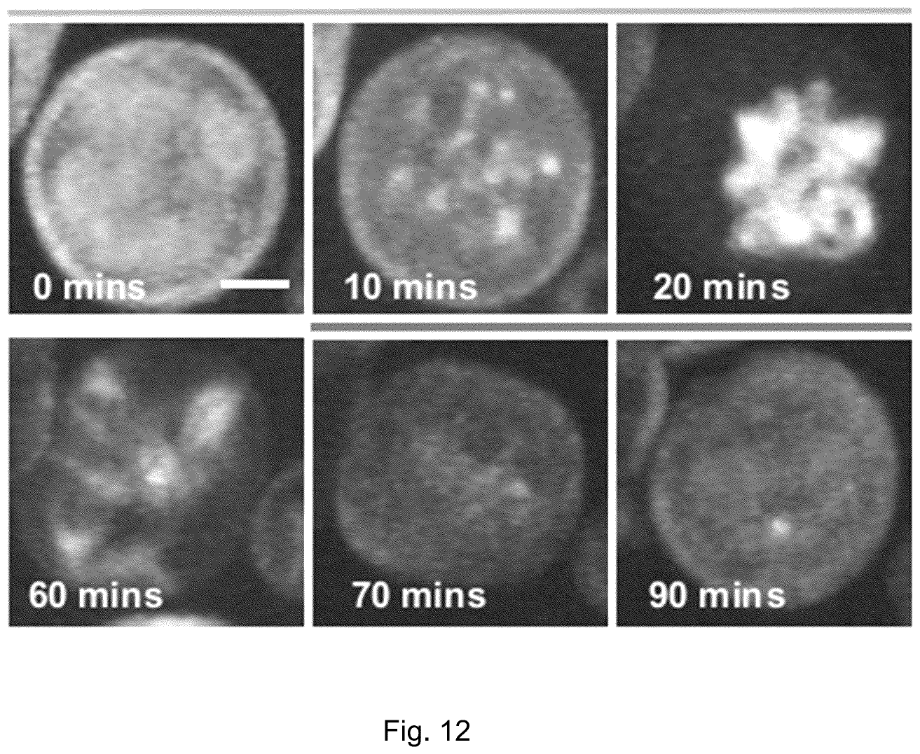

An alternative/complementary approach can be used to quantify cell cycle length in MKs treated with DNA damaging agents. MKs can be transduced with MSCV expressing PIP—FUCCI, a fluorescent ubiquitination cell cycle indicator (MSCV-FUCCI) 46 where MKs express proteins fused to mCherry (geminin, red) and/or m Venus (Cdt, green) depending on their cell cycle stage (FIG. 11). 46 This method has been used to quantify cell cycle length in various cells, including MKs. 47

If Niraparib is toxic in Tpo−/− or cMpl−/− mice due to already low platelet counts, in vitro assays can be used to 1) cultured bone marrow HSPCs from Tpo−/− and cMpl−/− mice and 2) CD34+ cells with TPO and cMPL deleted by CRISPR/Cas9. Murine and human cells can be treated with Niraparib and compounds outlined in Table 1 and the impact of loss of TPO/MPL on the differentiation of HSPCs into CD41+ cells in vitro quantified.

### REFERENCES

1. Novershtern N, Subramanian A, Lawton L N, Mak R H, Haining W N, McConkey M E, Habib N, Yosef N, Chang C Y, Shay T, et al. Densely interconnected transcriptional circuits control cell states in human hematopoiesis. Cell. 2011; 144:296-309. doi: 10.1016/j.cell.2011.01.004
2. Provan D, Arnold D M, Bussel J B, Chong B H, Cooper N, Gernsheimer T, Ghanima W, Godeau B, González-López T J, Grainger J, et al. Updated international consensus report on the investigation and management of primary immune thrombocytopenia. Blood Adv. 2019; 3:3780-3817. doi: 10.1182/bloodadvances.2019000812
3. Ghanima W, Geyer J T, Lee C S, Boiocchi L, Imahiyerobo A A, Orazi A, Bussel J B. Bone marrow fibrosis in 66 patients with immune thrombocytopenia treated with thrombopoietin-receptor agonists: a single-center, long-term follow-up. Haematologica. 2014; 99:937-944. doi: 10.3324/haematol.2013.098921
4. Mitchell W B, Bussel J B. Thrombopoietin Receptor Agonists: A Critical Review. Seminars in Hematology. 2015; 52:46-52.
5. Whitaker B, Rajbhandary S, Kleinman S, Harris A, Kamani N. Trends in United States blood collection and transfusion: results from the 2013 AABB Blood Collection, Utilization, and Patient Blood Management Survey. Transfusion. 2016; 56:2173-2183. doi: 10.1111/trf.13676
6. Hofmann A, Ozawa S, Shander A. Activity-based cost of platelet transfusions in medical and surgical inpatients at a US hospital. Vox Sanguinis. 2021; 116:998-1004.
7. Jackson S P, Bartek J. The DNA-damage response in human biology and disease. Nature. 2009; 461:1071-1078. doi: 10.1038/nature08467
8. Rose M, Burgess J T, O'Byrne K, Richard D J, Bolderson E. PARP Inhibitors: Clinical Relevance, Mechanisms of Action and Tumor Resistance. Front Cell Dev Biol. 2020; 8:564601. doi: 10.3389/fcell.2020.564601
9. Mateo J, Lord C J, Serra V, Tutt A, Balmana J, Castroviejo-Bermejo M, Cruz C, Oaknin A, Kaye S B, de Bono J S. A decade of clinical development of PARP inhibitors in perspective. Ann Oncol. 2019; 30:1437-1447. doi: 10.1093/annonc/mdz192
10. Pawlikowska P, Fouchet P, Vainchenker W, Rosselli F, Naim V. Defective endomitosis during megakaryopoiesis leads to thrombocytopenia in Fanca−/− mice. Blood. 2014; 124:3613-3623. doi: 10.1182/blood-2014-01-551457
11. Lu Y C, Sanada C, Xavier-Ferrucio J, Wang L, Zhang P X, Grimes H L, Venkatasubramanian M, Chetal K, Aronow B, Salomonis N, et al. The Molecular Signature of Megakaryocyte-Erythroid Progenitors Reveals a Role for the Cell Cycle in Fate Specification. Cell Rep. 2018; 25:2083-2093.e2084. doi: 10.1016/j.celrep.2018.10.084
12. Pietras E M, Reynaud D, Kang Y A, Carlin D, Calero-Nieto F J, Leavitt A D, Stuart J M, Gottgens B, Passegue E. Functionally Distinct Subsets of Lineage-Biased Multipotent Progenitors Control Blood Production in Normal and Regenerative Conditions. Cell stem cell. 2015; 17:35-46. doi: 10.1016/j.stem.2015.05.003
13. Sanjuan-Pla A, Macaulay I C, Jensen C T, Woll P S, Luis T C, Mead A, Moore S, Carella C, Matsuoka S, Bouriez Jones T, et al. Platelet-biased stem cells reside at the apex of the haematopoietic stem-cell hierarchy. Nature. 2013; 502:232-236. doi: 10.1038/nature 12495
14. Haas S, Hansson J, Klimmeck D, Loeffler D, Velten L, Uckelmann H, Wurzer S, Prendergast A M, Schnell A, Hexel K, et al. Inflammation-Induced Emergency Megakaryopoiesis Driven by Hematopoietic Stem Cell-like Megakaryocyte Progenitors. Cell stem cell. 2015; 17:422-434. doi: 10.1016/j.stem.2015.07.007
15. Machlus K R, Thon J N, Italiano J E, Jr. Interpreting the developmental dance of the megakaryocyte: a review of the cellular and molecular processes mediating platelet formation. Br J Haematol. 2014; 165:227-236. doi: 10.1111/bjh.12758
16. Noetzli L J, French S L, Machlus K R. New Insights Into the Differentiation of Megakaryocytes From Hematopoietic Progenitors. Arterioscler Thromb Vasc Biol. 2019; 39:1288-1300. doi: 10.1161/ATVBAHA.119.312129
17. Huang R, Zhou P K. DNA damage repair: historical perspectives, mechanistic pathways and clinical translation for targeted cancer therapy. Signal Transduct Target Ther. 2021; 6:254. doi: 10.1038/s41392-021-00648-7
18. Yousefzadeh M, Henpita C, Vyas R, Soto-Palma C, Robbins P, Niedernhofer L. DNA damage-how and why we age? eLife. 2021; 10: e62852. doi: 10.7554/eLife.62852
19. Li N, Chen H, Wang J. DNA damage and repair in the hematopoietic system. Acta Biochim Biophys Sin (Shanghai). 2022; 54:847-857. doi: 10.3724/abbs.2022053
20. Hanahan D, Weinberg R A. Hallmarks of cancer: the next generation. Cell. 2011; 144:646-674. doi: 10.1016/j.cell.2011.02.013
21. Wang M, Brandt L T L, Wang X, Russell H, Mitchell E, Kamimae-Lanning A N, Brown J M, Dingler F A, Garaycoechea J I, Isobe T, et al. Genotoxic aldehyde stress prematurely ages hematopoietic stem cells in a p53-driven manner. Molecular cell. 2023; 83:2417-2433 e2417. doi: 10.1016/j.molcel.2023.05.035
22. Meza-Leon B, Gratzinger D, Aguilar-Navarro A G, Juarez-Aguilar F G, Rebel V I, Torlakovic E, Purton L E, Dorantes-Acosta E M, Escobar-Sanchez A, Dick J E, et al. Human, mouse, and dog bone marrow show similar mesenchymal stromal cells within a distinctive microenvironment. Exp Hematol. 2021; 100:41-51. doi: 10.1016/j.exphem.2021.06.006
23. Canela A, Sridharan S, Sciascia N, Tubbs A, Meltzer P, Sleckman B P, Nussenzweig A. DNA Breaks and End Resection Measured Genome-wide by End Sequencing. Molecular cell. 2016; 63:898-911. doi: 10.1016/j.molcel.2016.06.034
24. Scanlon V M, Thompson E N, Lawton B R, Kochugaeva M, Ta K, Mayday M Y, Xavier-Ferrucio J, Kang E, Eskow N M, Lu Y C, et al. Multiparameter analysis of timelapse imaging reveals kinetics of megakaryocytic erythroid progenitor clonal expansion and differentiation. Sci Rep. 2022; 12:16218. doi: 10.1038/s41598-022-19013-x
25. Fok W C, Niero E L O, Dege C, Brenner K A, Sturgeon C M, Batista L F Z. p53 Mediates Failure of Human Definitive Hematopoiesis in Dyskeratosis Congenita. Stem Cell Reports. 2017; 9:409-418. doi: 10.1016/j.stemcr.2017.06.015
26. Fok W C, Shukla S, Vessoni A T, Brenner K A, Parker R, Sturgeon C M, Batista L F Z.

Posttranscriptional modulation of TERC by PAPD5 inhibition rescues hematopoietic development in dyskeratosis congenita. Blood. 2019; 133:1308-1312. doi: 10.1182/blood-2018-11-885368
27. Jeong H C, Shukla S, Fok W C, Huynh T N, Batista L F Z, Parker R. USB1 is a miRNA deadenylase that regulates hematopoietic development. Science. 2023; 379:901-907. doi: 10.1126/science.abj8379
28. Vessoni A T, Zhang T, Quinet A, Jeong H C, Munroe M, Wood M, Tedone E, Vindigni A, Shay J W, Greenberg R A, et al. Telomere erosion in human pluripotent stem cells leads to ATR-mediated mitotic catastrophe. The Journal of cell biology. 2021; 220. doi: 10.1083/jcb.202011014
29. Barrachina M N, Pernes G, Becker I C, Allaeys I, Hirsch T I, Groeneveld D J, Khan A O, Freire D, Guo K, Carminita E, et al. Efficient megakaryopoiesis and platelet production require phospholipid remodelingand PUFA uptake through CD36. Nature Cardiovascular Research. 2023; 2:746-763. doi: 10.1038/s44161-023-00305-y
30. Sanada C, Xavier-Ferrucio J, Lu Y C, Min E, Zhang P X, Zou S, Kang E, Zhang M, Zerafati G, Gallagher P G, et al. Adult human megakaryocyte-erythroid progenitors are in the CD34+CD38mid fraction. Blood. 2016; 128:923-933. doi: 10.1182/blood-2016-01-693705
31. Lu Y C, Sanada C, Xavier-Ferrucio J, Wang L, Zhang P X, Grimes H L, Venkatasubramanian M, Chetal K, Aronow B, Salomonis N, et al. The Molecular Signature of Megakaryocyte-Erythroid Progenitors Reveals a Role for the Cell Cycle in Fate Specification. Cell Rep. 2018; 25:2083-2093 e2084. doi: 10.1016/j.celrep.2018.10.084
32. Ayyar S, Beerman I. Detection of DNA Damage in Hematopoietic Stem Cells. Methods Mol Biol. 2023; 2567:11-28. doi: 10.1007/978-1-0716-2679-5_2
33. Jia N, Guo C, Nakazawa Y, van den Heuvel D, Luijsterburg M S, Ogi T. Dealing with transcription-blocking DNA damage: Repair mechanisms, RNA polymerase II processing and human disorders. DNA Repair (Amst). 2021; 106:103192. doi: 10.1016/j.dnarep.2021.103192
34. Marechal A, Zou L. RPA-coated single-stranded DNA as a platform for post-translational modifications in the DNA damage response. Cell research. 2015; 25:9-23. doi: 10.1038/cr.2014.147
35. Soniat M M, Myler L R, Kuo H C, Paull T T, Finkelstein I J. RPA Phosphorylation Inhibits DNA Resection. Molecular cell. 2019; 75:145-153 e145. doi: 10.1016/j.molcel.2019.05.005
36. Quinet A, Carvajal-Maldonado D, Lemacon D, Vindigni A. DNA Fiber Analysis: Mind the Gap! Methods Enzymol. 2017; 591:55-82. doi: 10.1016/bs.mie.2017.03.019
37. Moore G, Jimenez Sainz J, Jensen R B. DNA fiber combing protocol using in-house reagents and coverslips to analyze replication fork dynamics in mammalian cells. STAR Protoc. 2022; 3:101371. doi: 10.1016/j.xpro.2022.101371
38. Thongon N, Ma F, Santoni A, Marchesini M, Fiorini E, Rose A, Adema V, Ganan-Gomez I, Groarke E M, Gutierrez-Rodrigues F, et al. Hematopoiesis under telomere attrition at the single-cell resolution. Nat Commun. 2021; 12:6850. doi: 10.1038/s41467-021-27206-7
39. Revy P, Kannengiesser C, Bertuch A A. Genetics of human telomere biology disorders. Nat Rev Genet. 2023; 24:86-108. doi: 10.1038/s41576-022-00527-z
40. de Lange T. Shelterin-Mediated Telomere Protection. Annu Rev Genet. 2018; 52:223-247. doi: 10.1146/annurev-genet-032918-021921
41. Montenont E, Bhatlekar S, Jacob S, Kosaka Y, Manne B K, Lee O, Parra-Izquierdo I, Tugolukova E, Tolley N D, Rondina M T, et al. CRISPR-edited megakaryocytes for rapid screening of platelet gene functions. Blood Adv. 2021; 5:2362-2374. doi: 10.1182/bloodadvances.2020004112
42. Josefsson E C, James C, Henley K J, Debrincat M A, Rogers K L, Dowling M R, White M J, Kruse E A, Lane R M, Ellis S, et al. Megakaryocytes possess a functional intrinsic apoptosis pathway that must be restrained to survive and produce platelets. J Exp Med. 2011; 208:2017-2031. doi: 10.1084/jem.20110750
43. Josefsson E C, Burnett D L, Lebois M, Debrincat M A, White M J, Henley K J, Lane R M, Moujalled D, Preston S P, O'Reilly L A, et al. Platelet production proceeds independently of the intrinsic and extrinsic apoptosis pathways. Nat Commun. 2014; 5:3455. doi: 10.1038/ncomms4455
44. Yan W X, Mirzazadeh R, Garnerone S, Scott D, Schneider M W, Kallas T, Custodio J, Wernersson E, Li Y, Gao L, et al. BLISS is a versatile and quantitative method for genome-wide profiling of DNA double-strand breaks. Nat Commun. 2017; 8:15058. doi: 10.1038/ncomms1505845. Tilburg J, Stone A P, Billingsley J M, Scoville D K, Pavenko A, Liang Y, Italiano J E, Jr., Machlus K R. Spatial transcriptomics of murine bone marrow megakaryocytes at single-cell resolution. Res Pract Thromb Haemost. 2023; 7:100158. doi: 10.1016/j.rpth.2023.100158
46. Zielke N, Edgar B A. FUCCI sensors: powerful new tools for analysis of cell proliferation. Wiley Interdiscip Rev Dev Biol. 2015; 4:469-487. doi: 10.1002/wdev.189
47. Trakala M, Rodriguez-Acebes S, Maroto M, Symonds C E, Santamaria D, Ortega S, Barbacid M, Mendez J, Malumbres M. Functional reprogramming of polyploidization in megakaryocytes. Developmental cell. 2015; 32:155-167. doi: 10.1016/j.devcel.2014.12.015

### Example 3

### PARP Inhibitors and the Influence of DNA Damage on Megakaryopoiesis

### Background

Megakaryocytes are classically characterized by a polyploid nucleus, able to contain up to 128N. It has recently been shown that induction of a tetraploid state in cancer cells can result in increased accumulation of DNA damage, specifically double strand breaks (DSBs).⁽¹⁾ It was hypothesized that megakaryocytes are more prone to DNA damage by virtue of their high ploidy. Poly-ADP-ribose-polymerase inhibitors (PARPi) are DNA damage repair inhibiting cancer drugs used predominantly in the treatment of breast cancer susceptibility gene 1 and 2 (BRCA1 and BRCA2) deficient tumors. These tumors have compromised DNA damage repair and so PARPi work via synthetic lethality. Notably, these drugs have highly documented hematological side-effects including thrombocytopenia.⁽²⁾. Without wishing to be bound by theory, it is contemplated herein that that PARPi induced thrombocytopenia may result from inhibition of the repair of naturally occurring damage in megakaryocytes resulting in reduced or defective megakaryocyte maturation.

### Objectives

It was determined if thrombocytopenia in patients receiving PARPi is a consequence of effects on platelet reactivity, if the commercially available PARPi Olaparib, Niraparib and Rucaparib cause a reduction in megakaryopoiesis in vitro and in vivo, and if megakaryocytes accumulate DNA damage during normal maturation and whether damage scales with ploidy level.

### Results

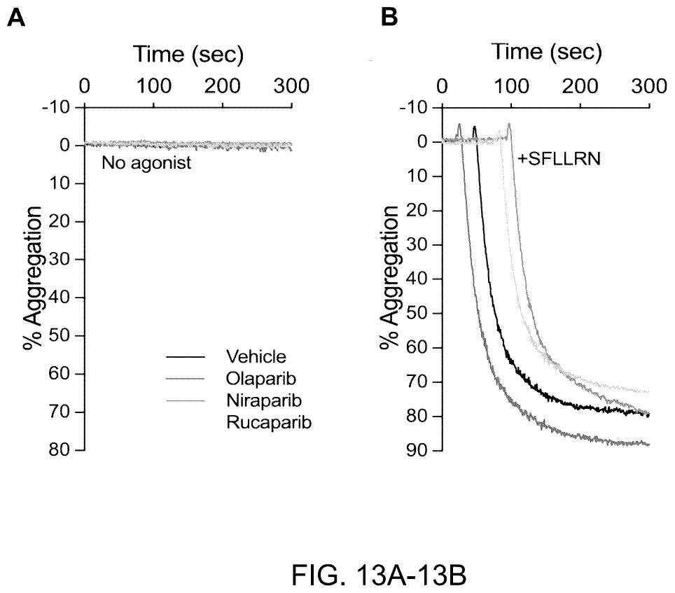

PARPi do not affect platelet aggregation in vitro. Platelet aggregation measured after incubation of human platelet rich plasma with PARPi (FIG. 13B) or DMSO (FIG. 13A) vehicle control.

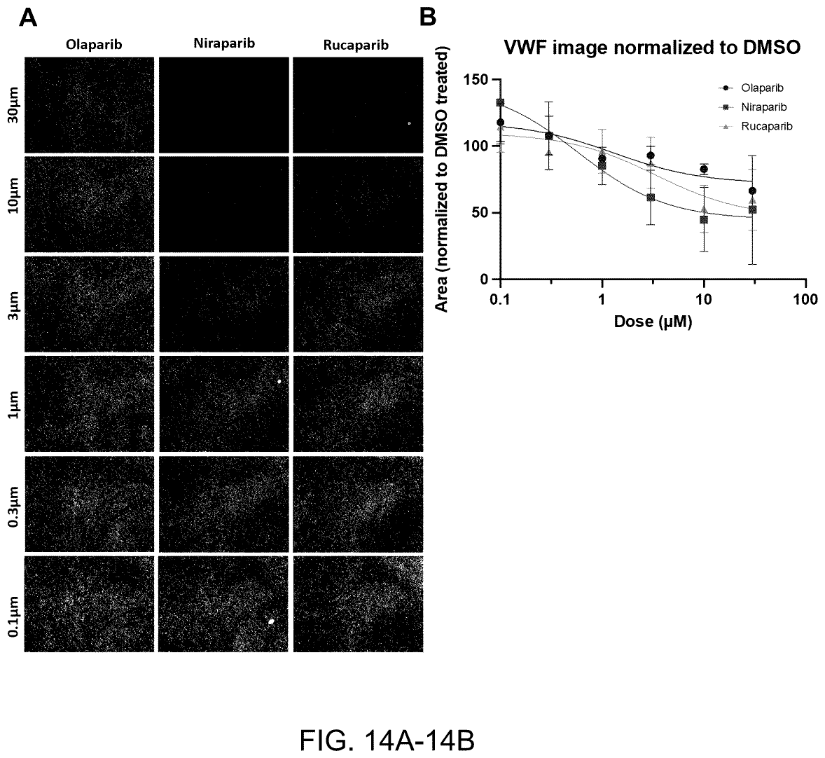

PARPi reduces megakaryocyte differentiation in vitro. Lineage depleted bone marrow was cultured with TPO for 4 days in the presence of PARPi or vehicle controls (FIG. 14A-14B).

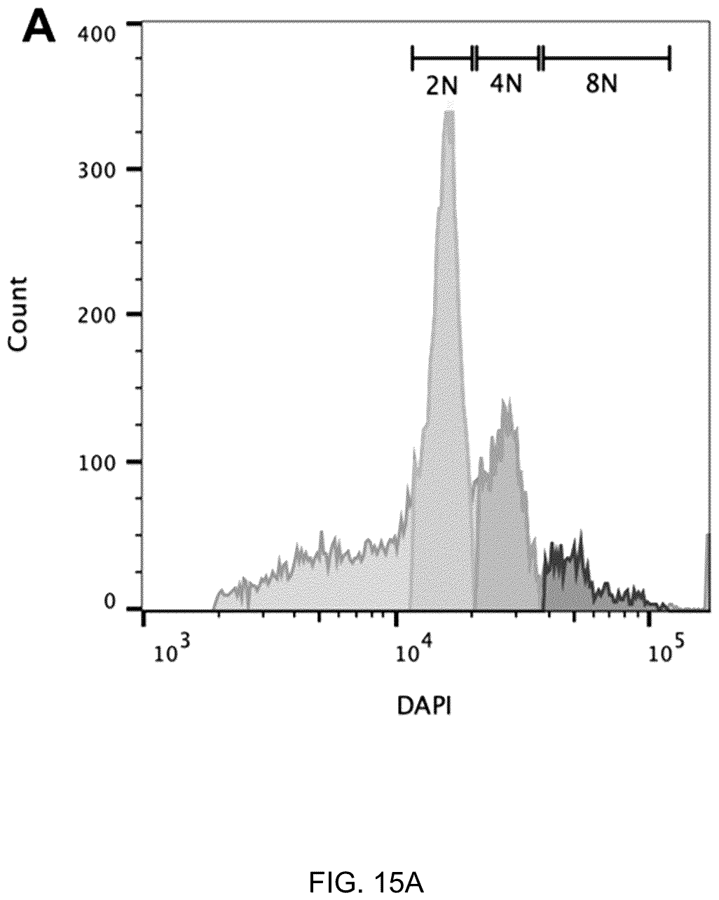

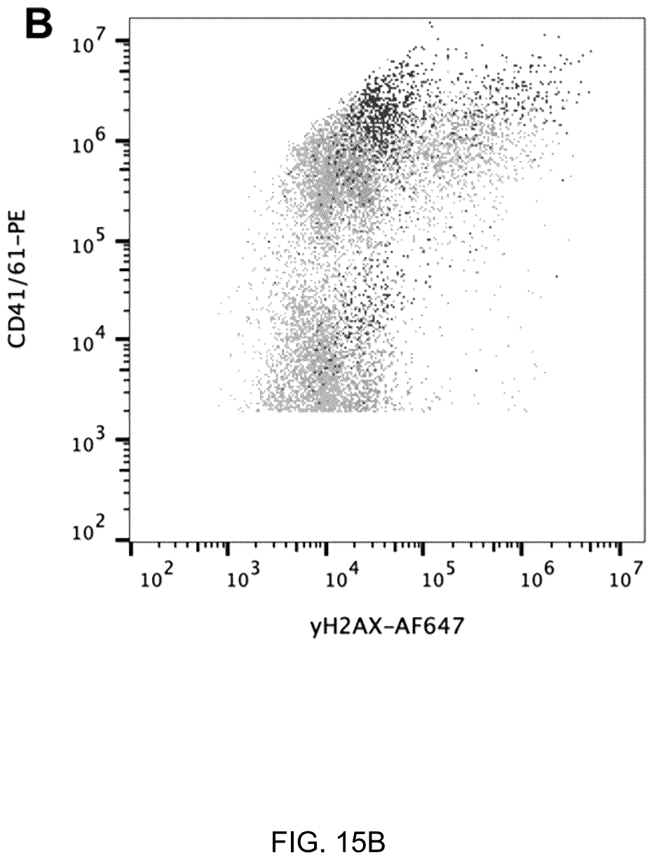

Megakaryocytes accrue DSBs in vitro and this increases and damage increases with ploidy. Human BM CD34+ cells were differentiated into megakaryocytes in vitro. At Day 10, cells were assayed by flow cytometry for megakaryocyte differentiation (CD41+), ploidy, and DNA damage (FIG. 15-15B).

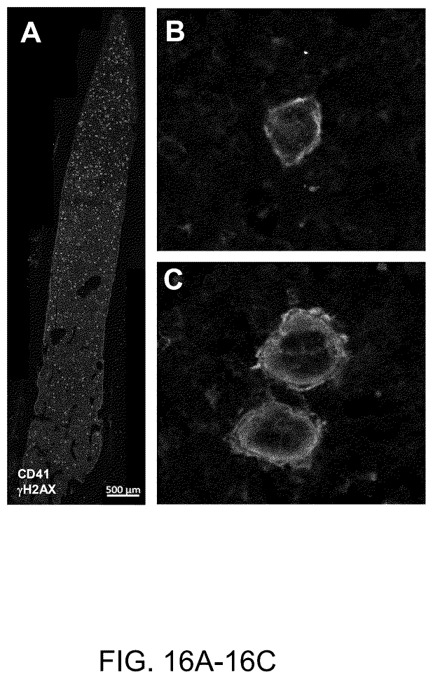

Immunofluorescence of mouse bone marrow reveals gH2AX foci and thus DSBs in megakaryocytes. Mouse femur sections were cut using the Kawamoto film method.⁽³⁾ Megakaryocytes and DNA DSBs were immunolabelled with anti-CD41 and anti-gH2AX, respectively. After mounting, slides were imaged on a Zeiss LSM 880 Confocal microscope with a 40× objective (FIGS. 16A-16BC). gH2AX foci can be seen in megakaryocytes, indicating the presence of DSBs. Approximately 11% of megakaryocytes in this image contained foci.

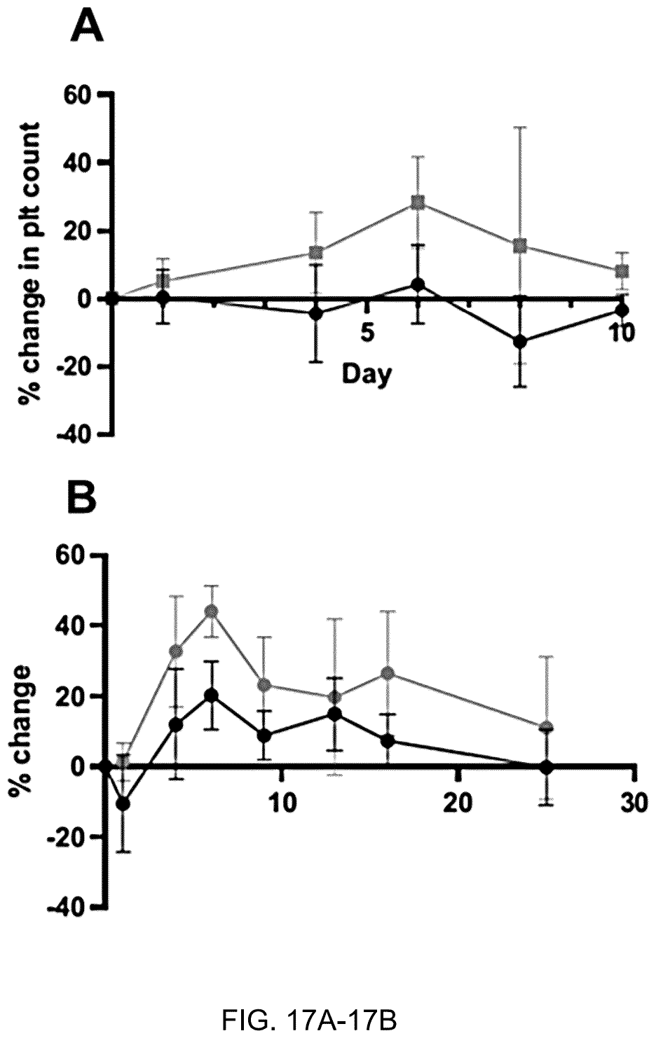

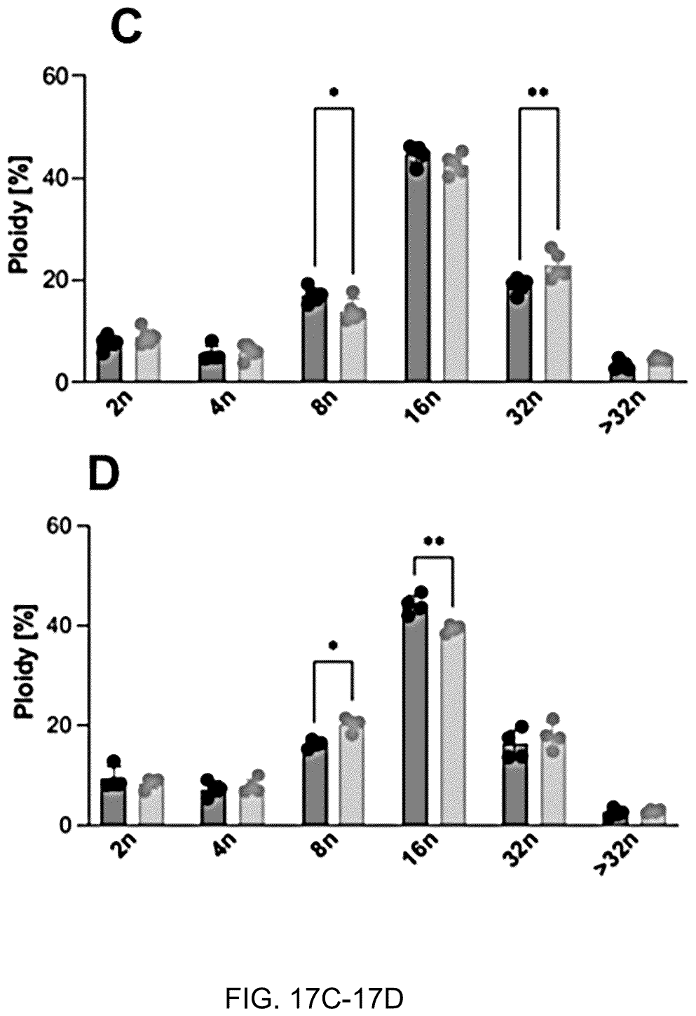

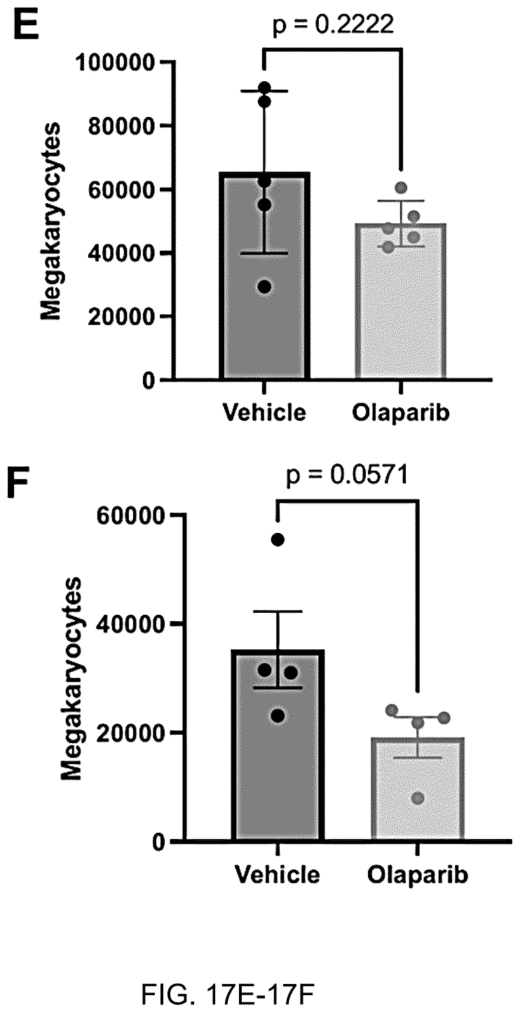

Olaparib treatment increases platelet count and alters megakaryocyte ploidy in mice. C57B1/6 mice were dosed with 50 mg/kg Olaparib intraperitoneally for 5 days consecutive days each week. In both 2-week (FIG. 17A) and 4-week (FIG. 17B) experiments, an increase was founding platelet count at day 6 before platelet counts returned to approximately baseline. After 2-weeks megakaryocyte ploidy shifted to fewer 8N and more 32N cells (FIG. 17C), whilst after 4-weeks an opposite shift to more 8N and fewer 16N cells was identified (FIG. 17D). The number of megakaryocytes detected by CD41+CD42+ flow cytometric staining of bone marrow cells revealed a trend to fewer megakaryocytes in the Olaparib treatment at both 2-weeks (FIG. 17E) and 4-weeks (FIG. 17F).

### Discussion

All three clinically used PARPi reduced megakaryocyte differentiation in vitro, but do not impact platelet reactivity. In vitro differentiated megakaryocytes have more DSBs as their ploidy increases. Megakaryocytes can be found with DSBs in vivo. In vivo application of Olaparib resulted in a surprising increase in platelet count with changes in ploidy changing relative to the length of treatment.

### REFERENCES

1 LaFargue, C. J., et al., Exploring and comparing adverse events between PARP inhibitors. Lancet Oncol, 2019. 20 (1): p. e15-e28.
2 Gemble, S., et al., Genetic instability from a single S phase after whole-genome duplication. Nature, 2022. 604 (7904): p. 146-151.
3 Kawamoto, T. and M. Shimizu, A method for preparing 2- to 50-μm-thick fresh-frozen sections of large samples and undecalcified hard tissues. Histochemistry and Cell Biology, 2000. 113 (5): p. 331-339.

### Example 4

DNA Damage Accumulation in Polyploid Megakaryocytes: Investigating the Impact of Poly(ADP-ribose) polymerase (PARP) Inhibitors

### Background

Megakaryocytes are large hematopoietic cells that can reach ploidy levels greater than 64n. The potential for DNA damage accumulation in polyploid megakaryocytes is of interest as cancer cells with a DNA complement of even 4N are more susceptible to DNA damage. Poly(ADP-ribose) polymerase (PARP) inhibitors exhibit anti-tumor effects in solid tumors through induction of single stranded DNA breaks, ultimately leading to double stranded breaks and cell death. Hematological side effects including thrombocytopenia are the main dose-limiting toxicity in the clinic. However, the molecular mechanisms behind this thrombocytopenia remain largely unknown.

Aims: Investigate the potential for DNA damage accumulation in megakaryocytes and understand the effect of PARP inhibitor-induced thrombocytopenia by assessing megakaryocytic function and platelet production.

Methods: Immunofluorescence staining of yH2AX, a marker of DNA damage, was performed on native and in-vitro matured murine and human megakaryocytes. The effect of three clinically relevant PARP inhibitors was assessed on platelet functional assays and in vitro megakaryopoiesis. Finally, PARP inhibitors were administered to mice to investigate how they affect megakaryocyte differentiation, polyploidization, and platelet formation.

Results: Analysis of wildtype murine femurs revealed that megakaryocytes were positive for γH2AX staining (FIG. 13A-13B). Human megakaryocytes, differentiated from bone marrow derived CD34+ cells, similarly showed yH2AX foci. Increased megakaryocyte yH2AX was associated with higher ploidy. Addition of PARP inhibitors to murine lineage depleted bone marrow cells resulted in reduced megakaryopoiesis (FIG. 14A) and decreased megakaryocyte PARylation. Platelet function, however, remained intact (Figure FIG. 14B-14C). In vivo application of PARP inhibitors to healthy C57BL/6J mice led to a significant shift toward higher ploidy megakaryocytes (>32n).

Discussion: The data provided herein demonstrates that higher ploidy correlates with increased DNA damage and PARP inhibitors may exert their hematological toxicity through increasing DNA damage to megakaryocytes.

### Example 5

DNA Damage Accumulation in Polyploid Megakaryocytes: Investigating the Impact of Poly(ADP-ribose) polymerase (PARP) Inhibitors

### Introduction

Megakaryocytes are classically characterized by a polyploid nucleus, able to reach ploidy levels greater than 64n. The potential for DNA damage accumulation in polyploid megakaryocytes is of interest as cancer cells with a DNA complement of even 4N are more susceptible to DNA damage.¹ It was hypothesized that megakaryocytes are more prone to DNA damage by virtue of their high ploidy. Poly(ADP-ribose) polymerase (PARP) inhibitors exhibit anti-tumor effects in solid tumors through induction of single stranded DNA breaks, ultimately leading to double stranded breaks and cell death. Hematological side effects including thrombocytopenia are the main dose-limiting toxicity in the clinic. However, the molecular mechanisms behind this thrombocytopenia remain largely unknown.² It was hypothesized that PARPi induced thrombocytopenia results from inhibition of the repair of naturally occurring damage in megakaryocytes resulting in reduced or defective megakaryocyte maturation.

### Aims

It was determined if thrombocytopenia in patients receiving PARPi is a consequence of effects on platelet reactivity, and whether the commercially available PARPi olaparib, niraparib and rucaparib cause a reduction in megakaryopoiesis in vitro and in vivo, and if megakaryocytes accumulate DNA damage during normal maturation and whether damage scales with ploidy level.

### Results

PARP inhibitors do not affect platelet aggregation in vitro. Platelet aggregation was measured after incubation of human platelet rich plasma with PARPi or DMSO vehicle control (FIGS. 13A-13B).

PARPi reduce megakaryocyte differentiation in vitro. Lineage depleted murine bone marrow was cultured with TPO for 4 days in the presence of PARPi or vehicle controls (FIG. 14A-14B).

Megakaryocytes accrue DSBs in vitro and this increases with ploidy. Human BM CD34+ cells were differentiated into megakaryocytes in vitro. At Day 10, cells were assayed by flow cytometry for megakaryocyte differentiation (CD41+), ploidy, and DNA damage (FIG. 15A-15B). A concomitant increase in gH2AX signal was detected as ploidy increases.

Mouse megakaryocytes reveal gH2AX foci and thus DSBs. Mouse femur sections were cut using the Kawamoto film method.³ Megakaryocytes and DNA DSBs were immunolabelled with anti-CD41 and anti-gH2AX, respectively. After mounting, slides were imaged on a Zeiss LSM 880 Confocal microscope with a 40× objective (FIGS. 16A-16C). gH2AX foci can be seen in megakaryocytes, indicating the presence of DSBs. Approximately 11% of megakaryocytes in this image contained foci.

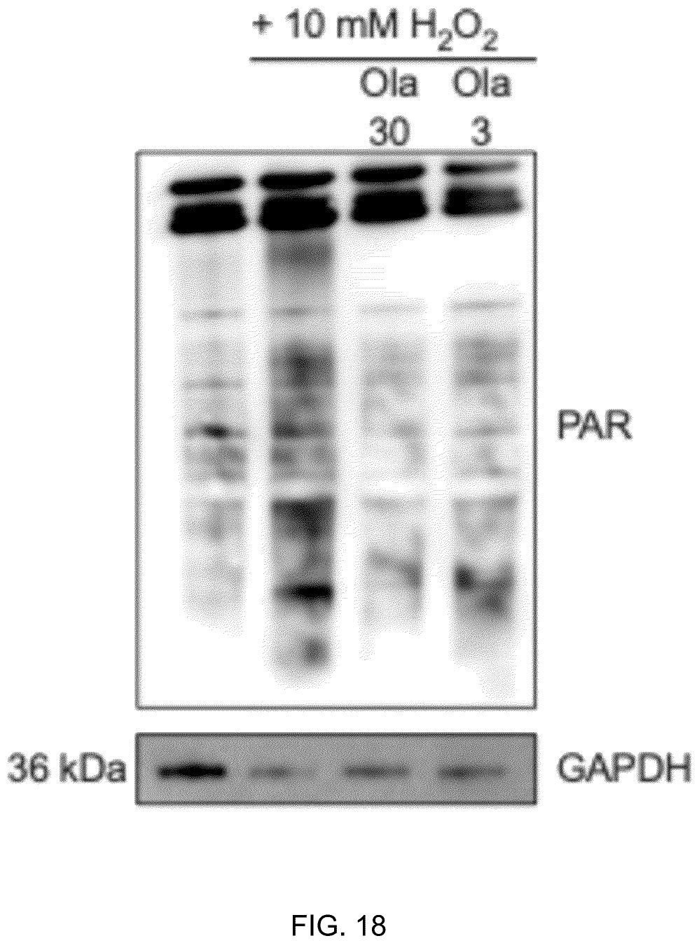

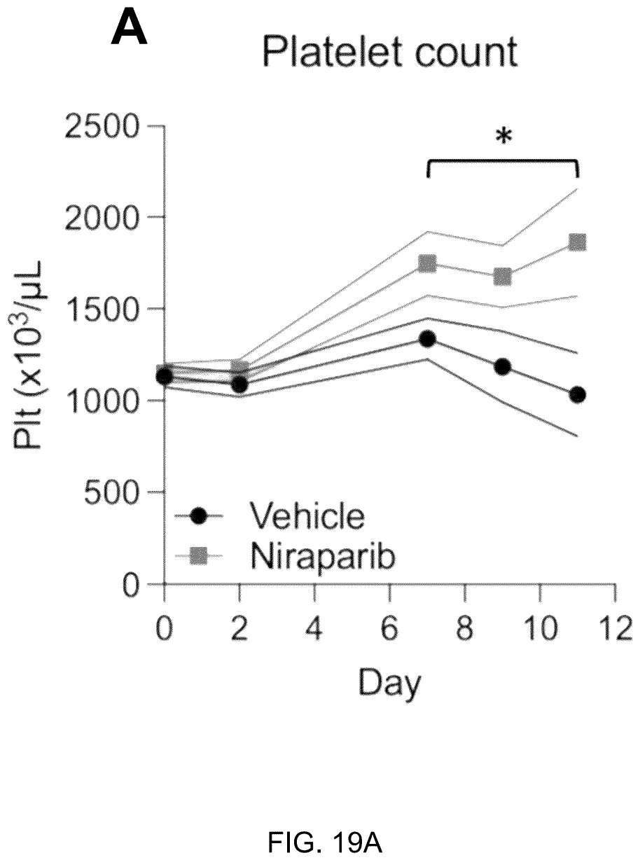

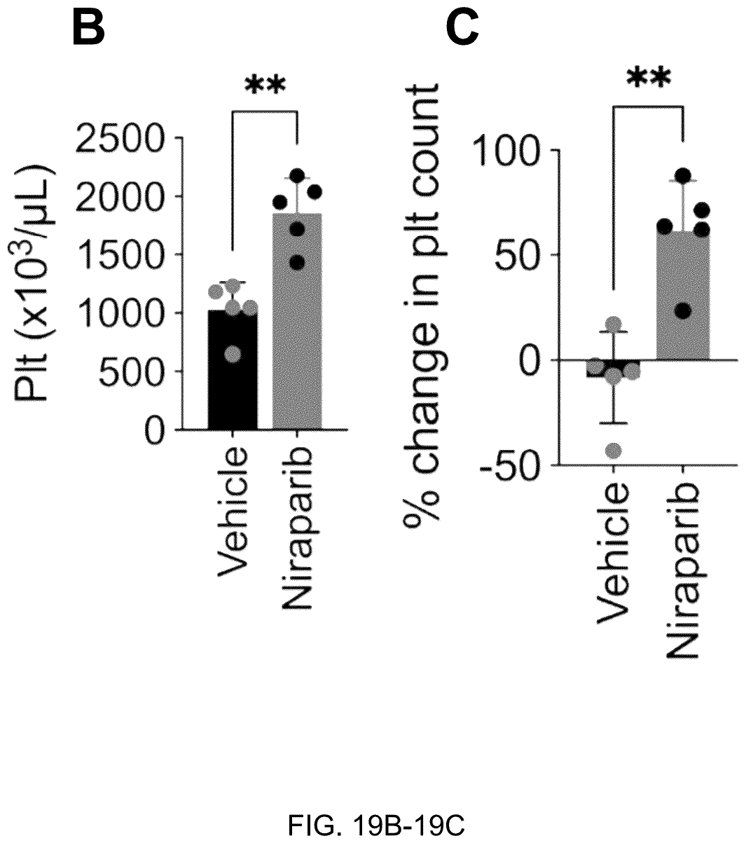

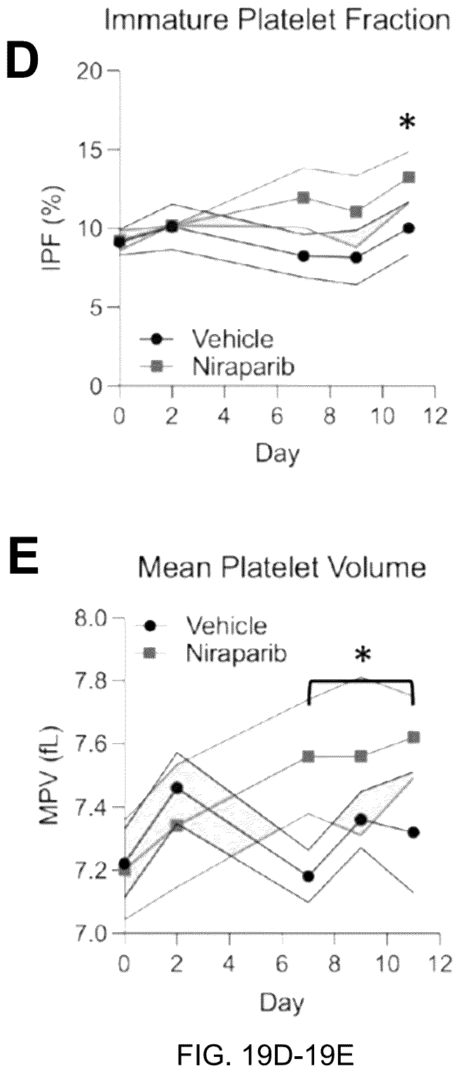

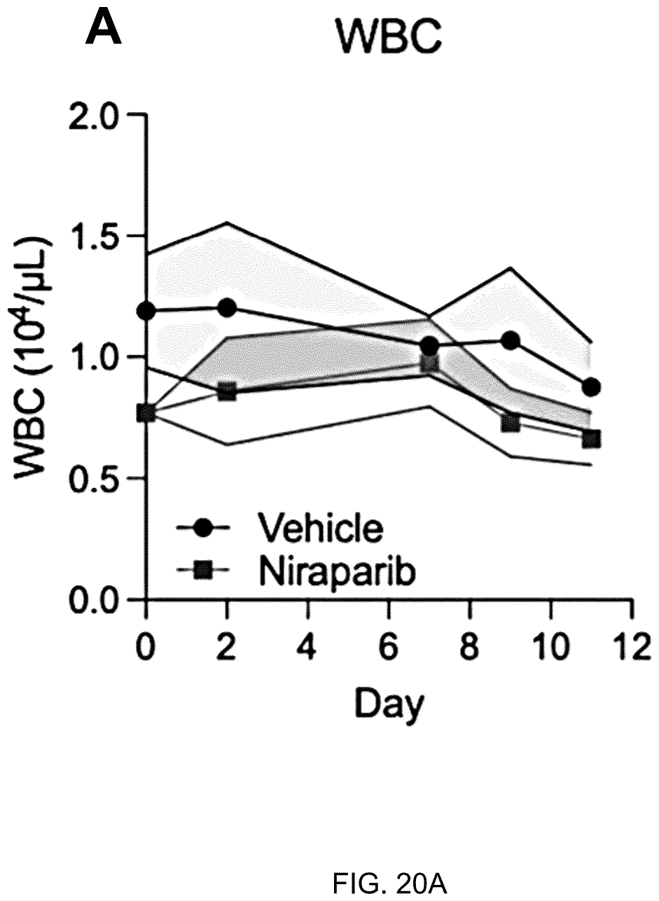

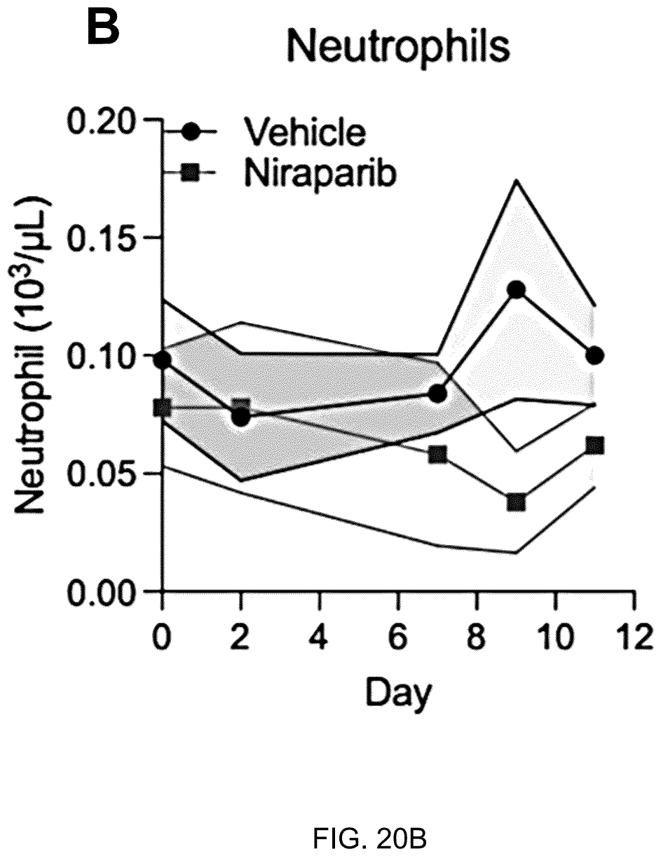

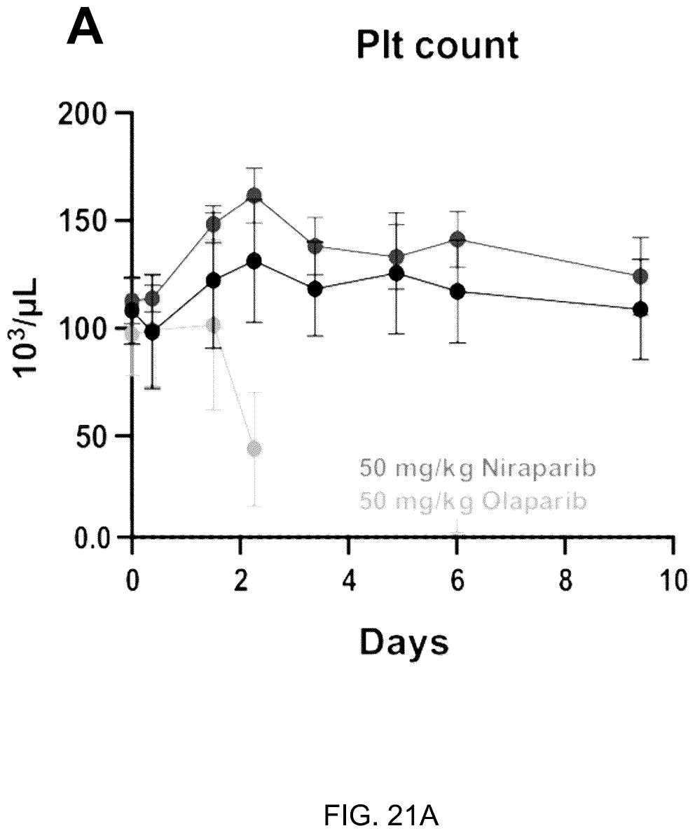

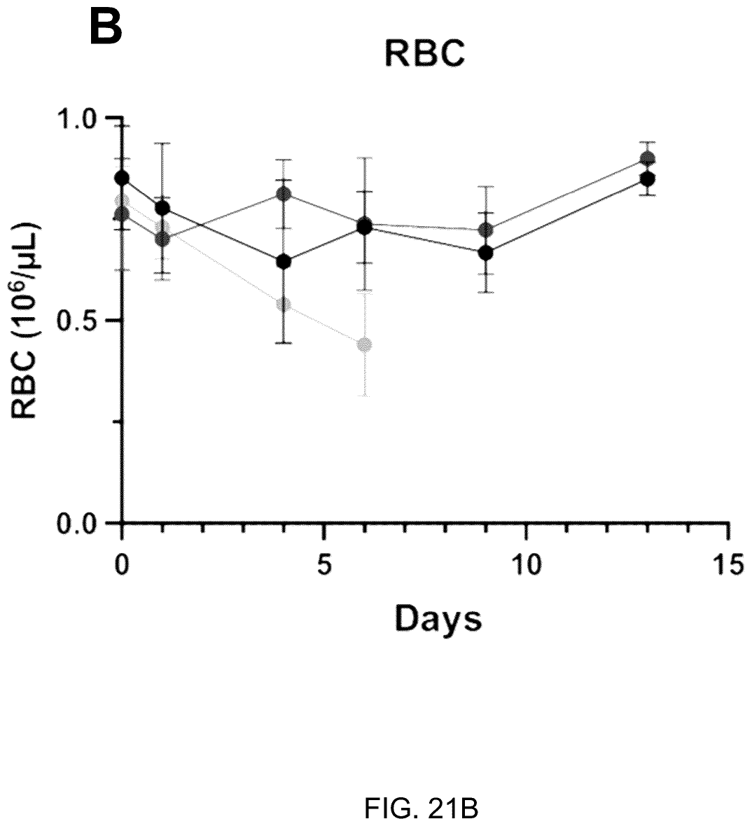

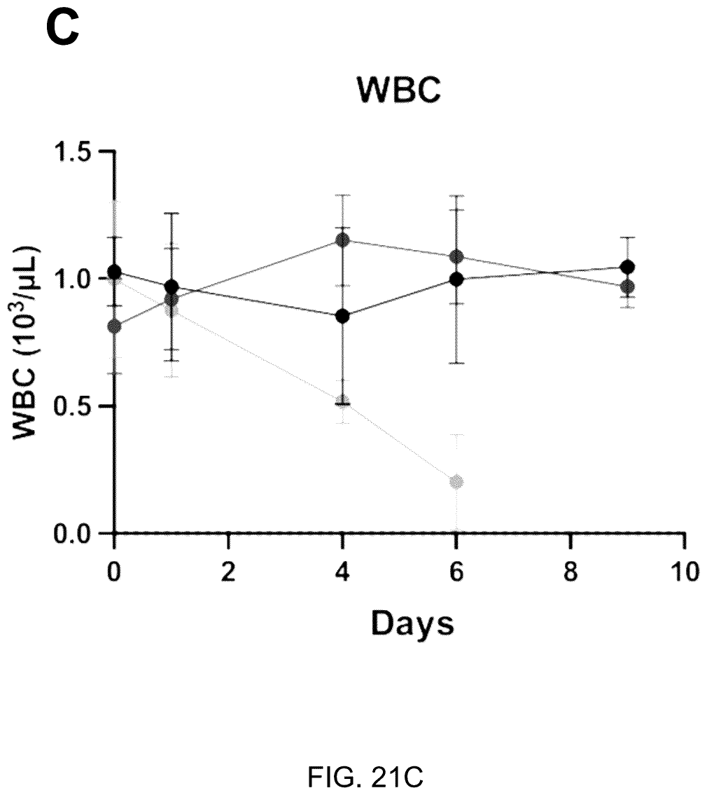

PARP inhibition reverses PARylation pattern in megakaryocytes induced by oxidative stress. Lineage depleted murine bone marrow was cultured with TPO for 4 days. Gradient-isolated megakaryocytes were subjected to 10 mM of hydrogen peroxide (H₂O₂) in the presence or absence of olaparib (30 μM or 3 μM). Cells were subsequently lysed in RIPA buffer containing protease inhibitor cocktail. A western blot to detect PARylation was performed (FIG. 18). Equal loading was confirmed using an anti-GAPDH antibody.

Olaparib treatment increases platelet count and alters megakaryocyte ploidy in mice. C57B1/6 mice were dosed with 50 mg/kg Olaparib intraperitoneally for 5 days consecutive days each week. In both 2-week (FIG. 17A) and 4-week (FIG. 17B) experiments, an increase in platelet count was found at day 6 before platelet counts returned to approximately baseline. After 2-weeks megakaryocyte ploidy shifted to fewer 8N and more 32N cells (FIG. 17C), whilst after 4-weeks an opposite shift to more 8N and fewer 16N cells was identified (FIG. 17D). The number of megakaryocytes detected by CD41+CD42+ flow cytometric staining of bone marrow cells revealed a trend to fewer megakaryocytes in the olaparib treatment at both 2-weeks (FIG. 17E) and 4-weeks (FIG. 17F).

### Discussion

All three clinically used PARPi reduced megakaryocyte differentiation in vitro, but do not impact platelet reactivity. In vitro differentiated megakaryocytes have more DSBs as their ploidy increases. Megakaryocytes can be found with DSBs in vivo. In vivo application of Olaparib resulted in a surprising increase in platelet count with changes in ploidy changing relative to the length of treatment.

### REFERENCES

1 LaFargue, C. J., et al., Exploring and comparing adverse events between PARP inhibitors. Lancet Oncol, 2019. 20 (1): p. e15-e28.
2 Gemble, S., et al., Genetic instability from a single S phase after whole-genome duplication. Nature, 2022. 604 (7904): p. 146-151.
3 Kawamoto, T. and M. Shimizu, A method for preparing 2- to 50-um-thick fresh-frozen sections of large samples and undecalcified hard tissues. Histochemistry and Cell Biology, 2000. 113 (5): p. 331-339.

### Example 6

PARP Inhibitors in the clinic lead to accumulation of DNA damage and often thrombocytopenia. It was determined what effect this had on MKs.

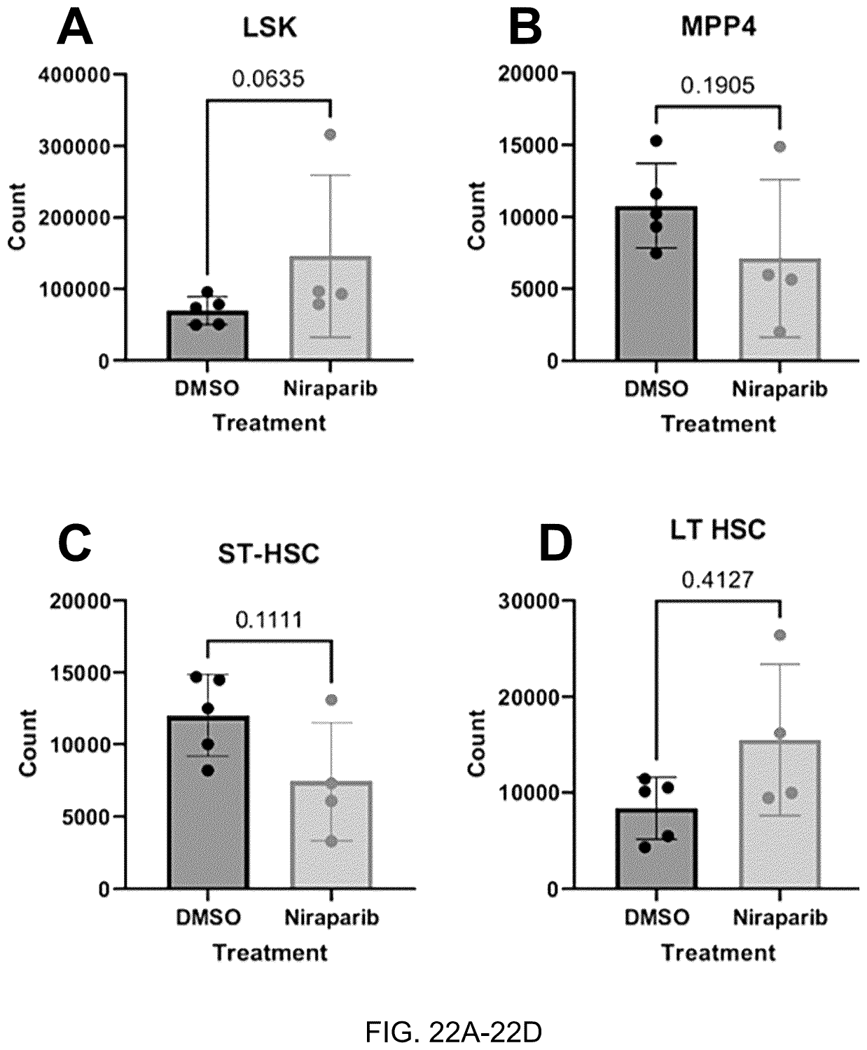

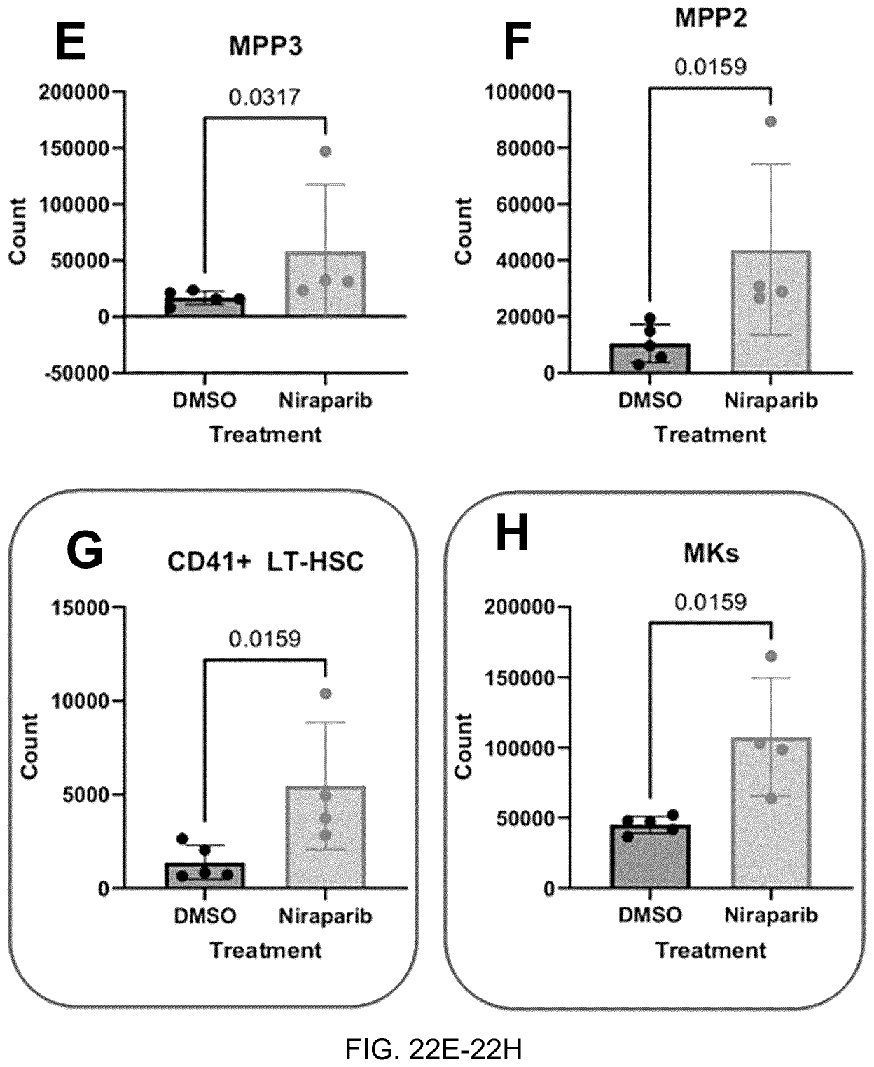

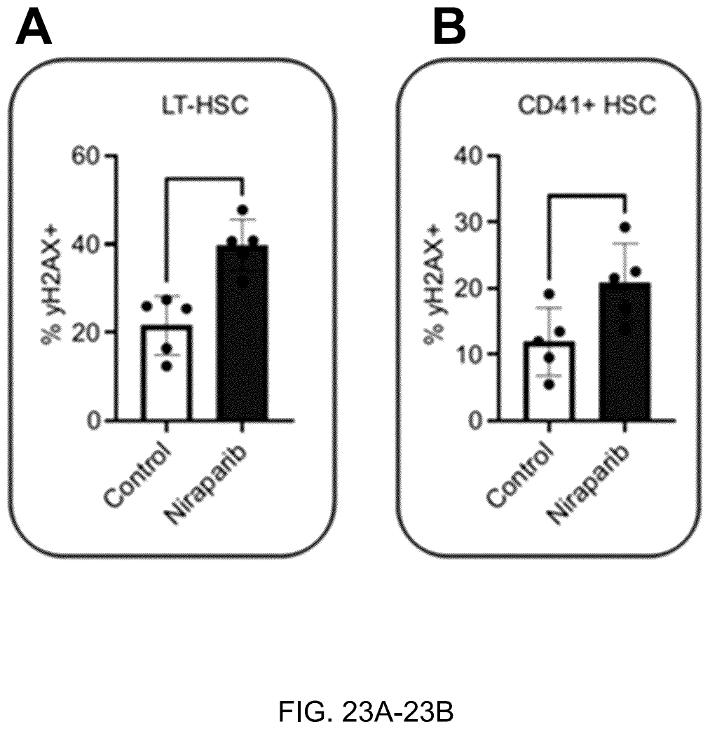

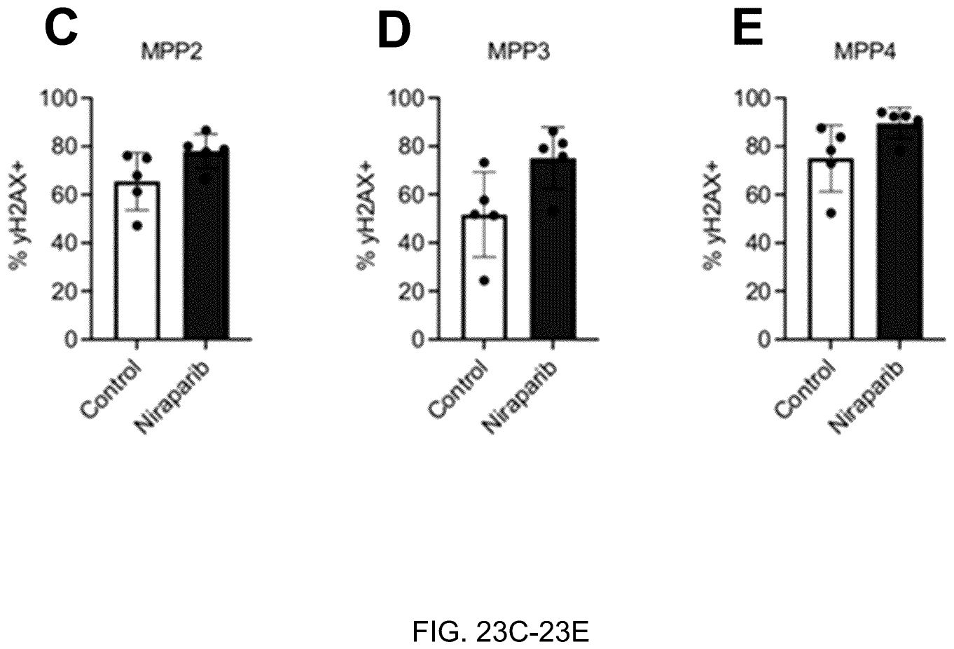

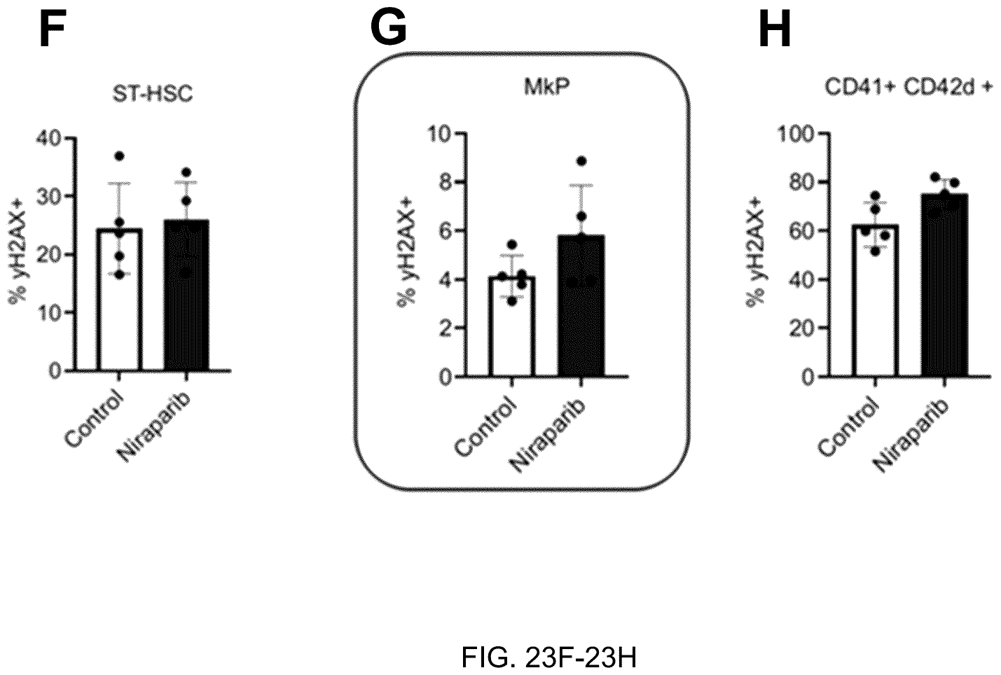

At Day 3, a HSPC/Myeloid Panel was performed and found a significant increase in MKs in BOTH committed progenitors AND classical hematopoiesis pathways, indicating activation of emergency and ‘normal’ hematopoiesis (FIGS. 22A-22H). At Day 3, 3 γH2AX Positivity was observed in the HSPC/Myeloid Panel, indicating that PARP inhibitor treatment induces DNA damage in MK-committed HSCs and MkPs (FIG. 23A-23H).

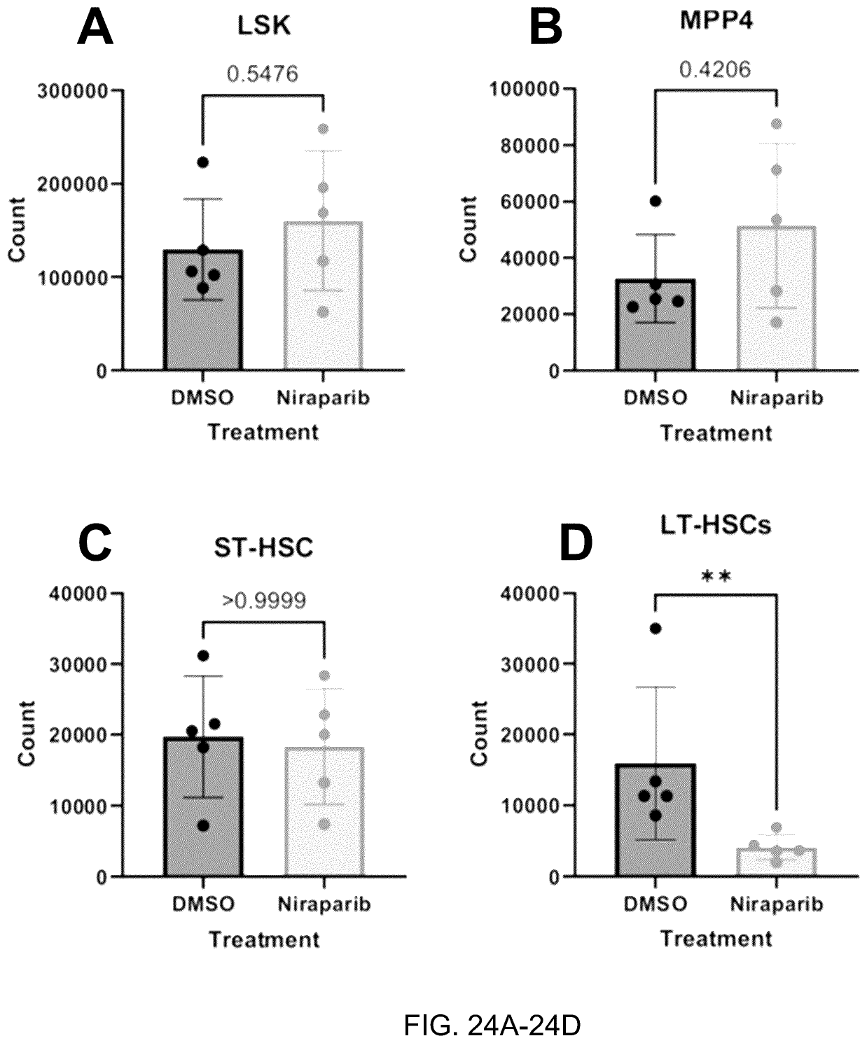

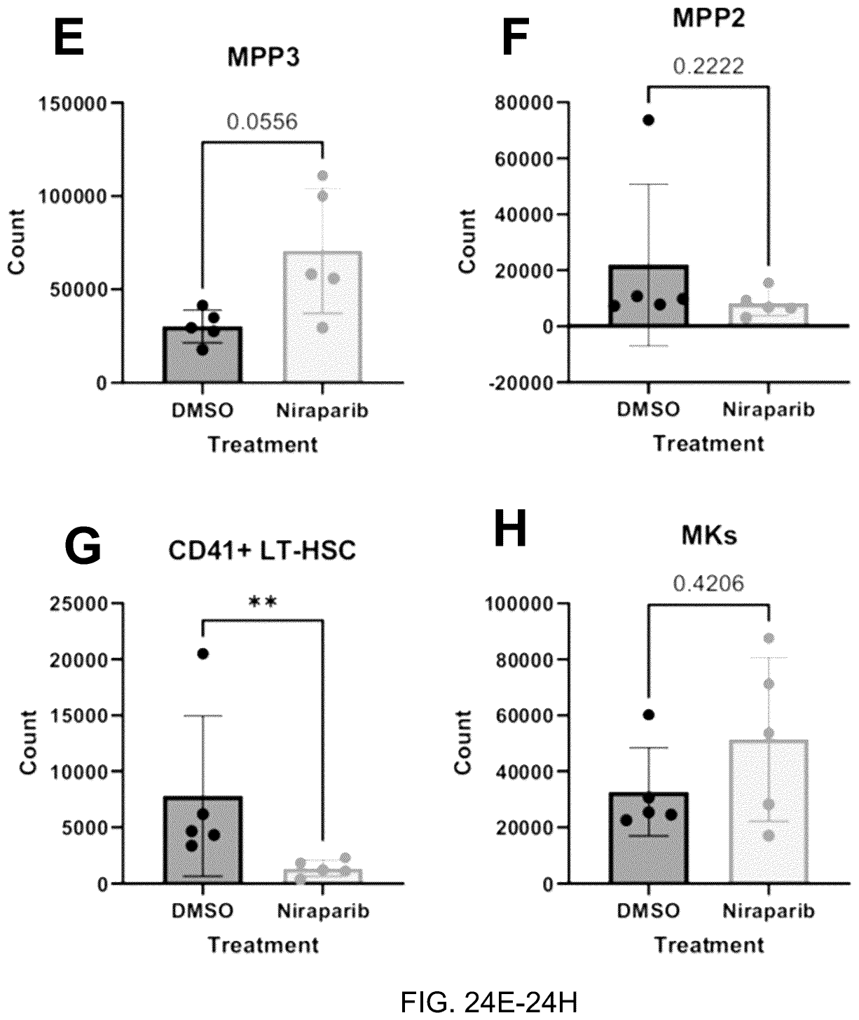

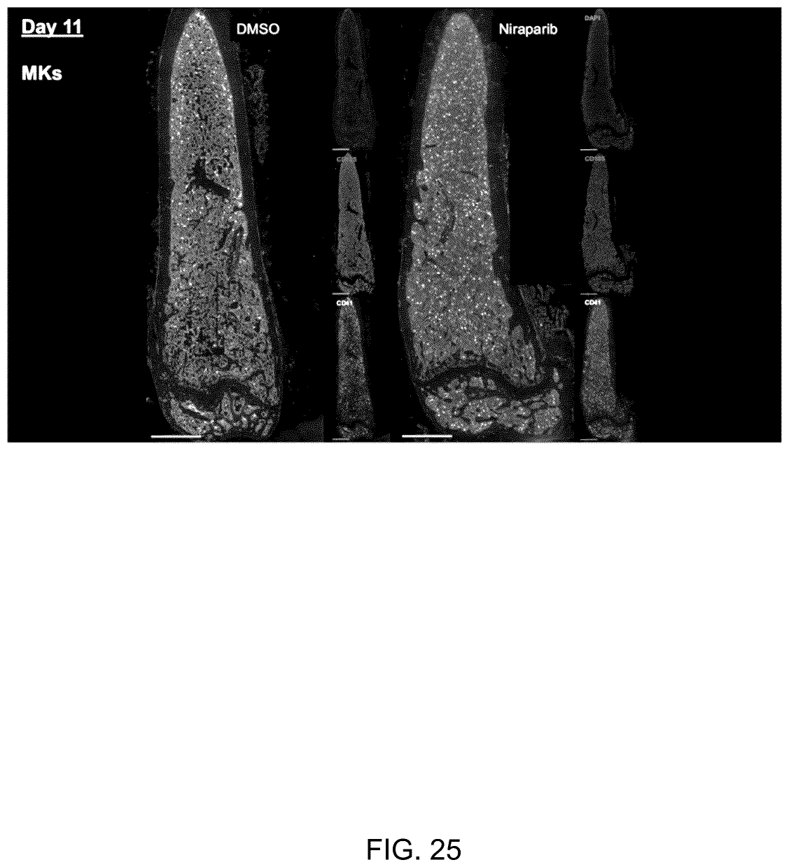

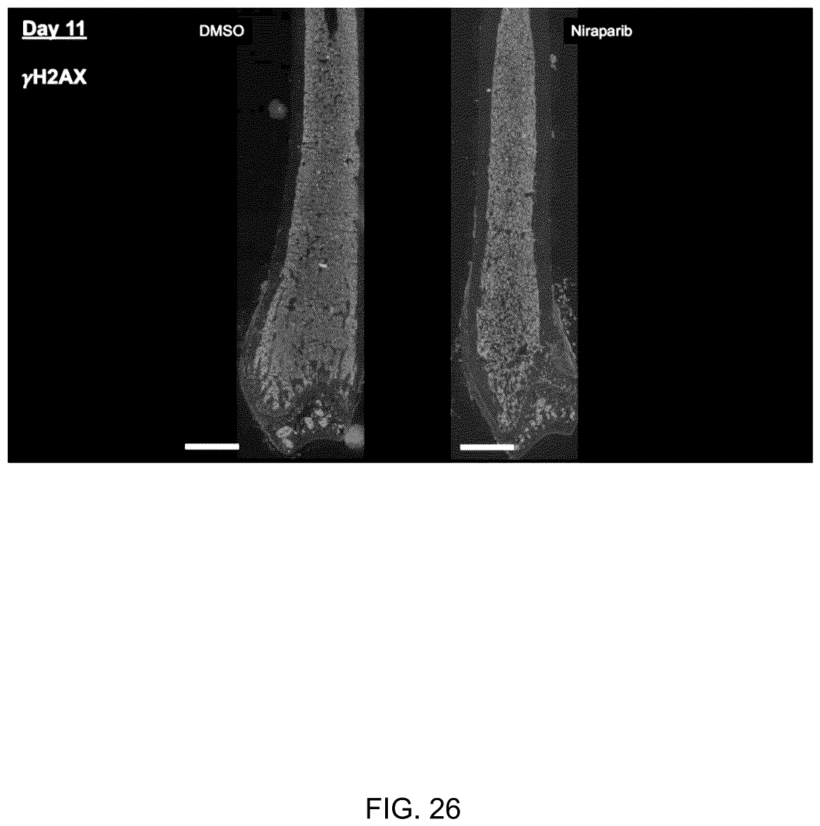

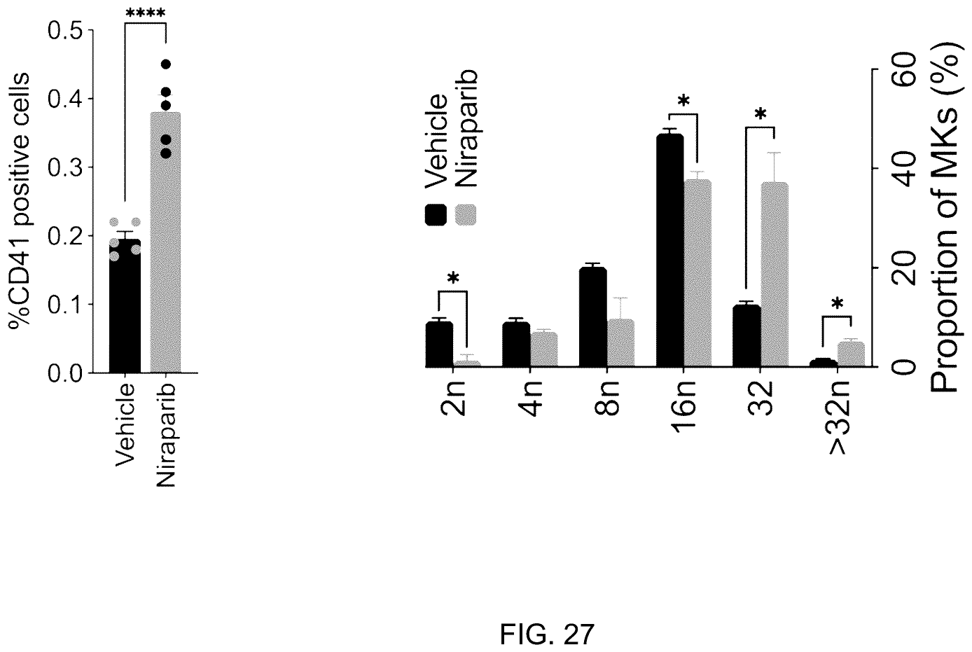

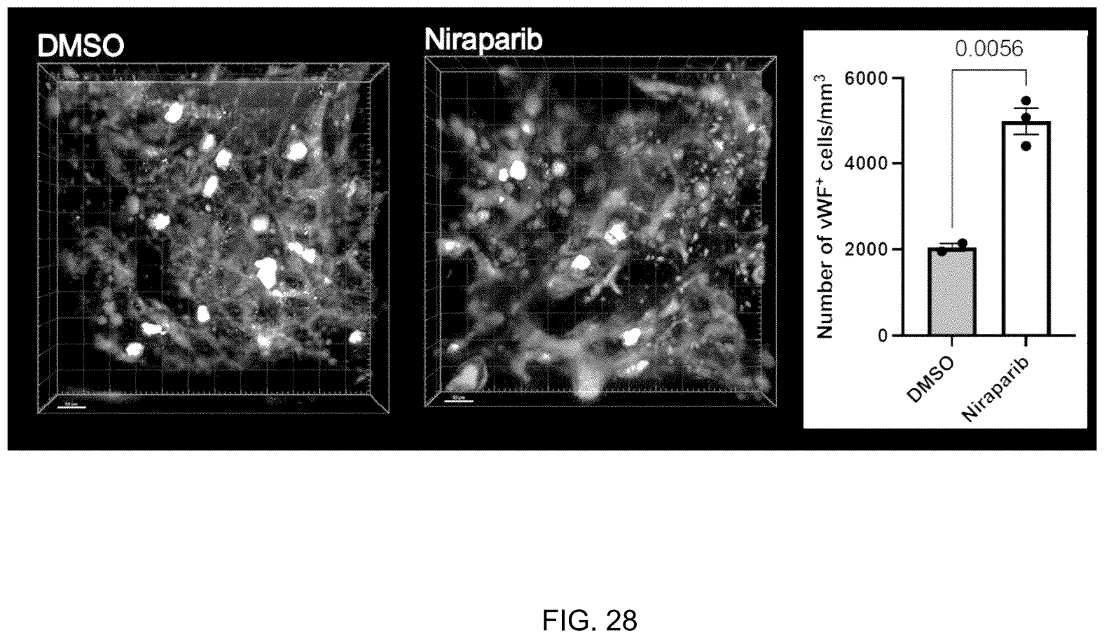

At Day 11 a HSPC/Myeloid Panel was performed. The results indicate signs of HSC exhaustion although MKs and platelets are still increased (FIGS. 24A-24H). Panels have been conducted as far as 30 days with no drop in platelet counts.

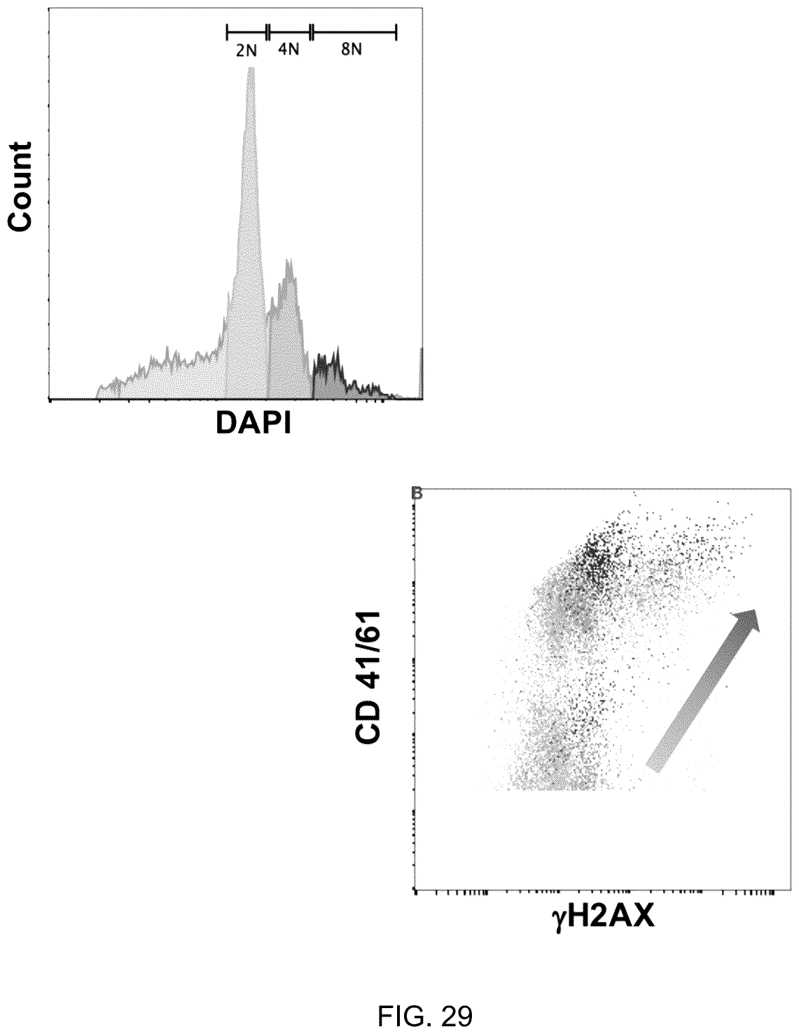

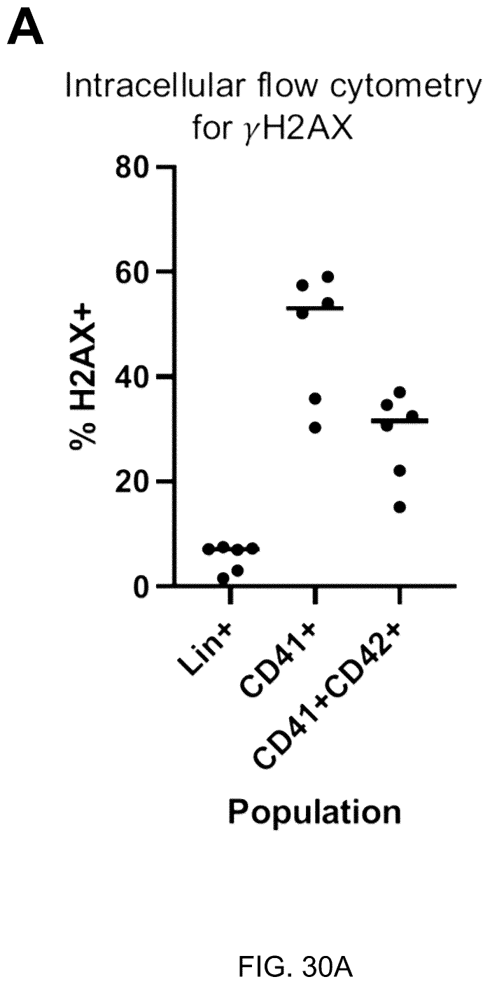

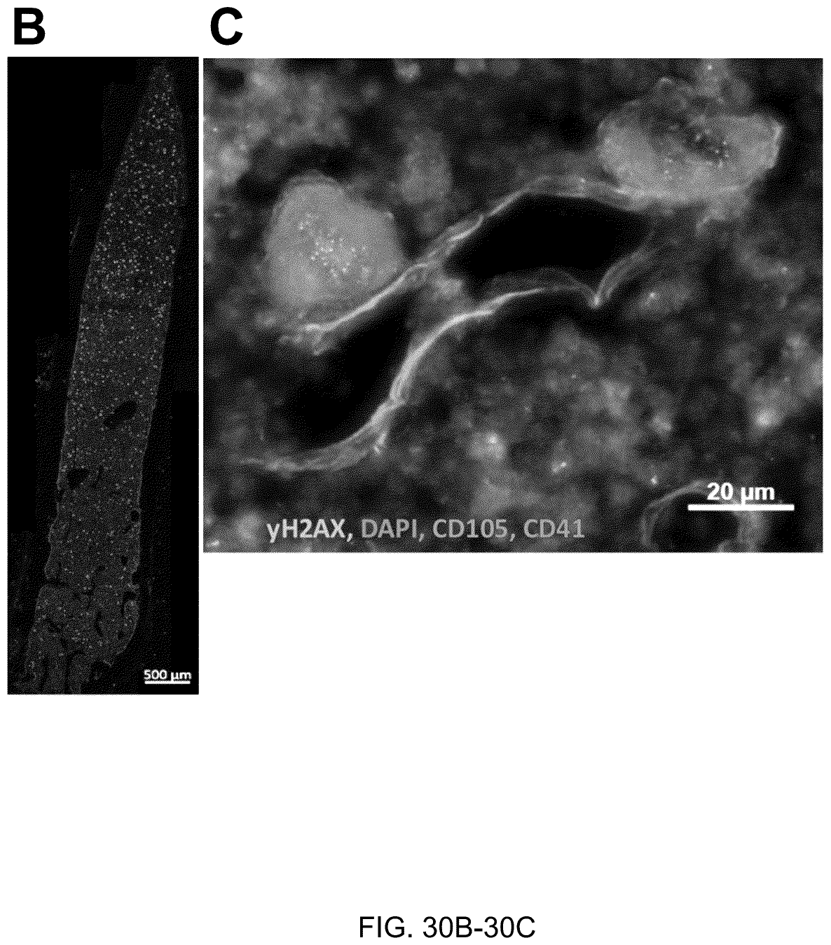

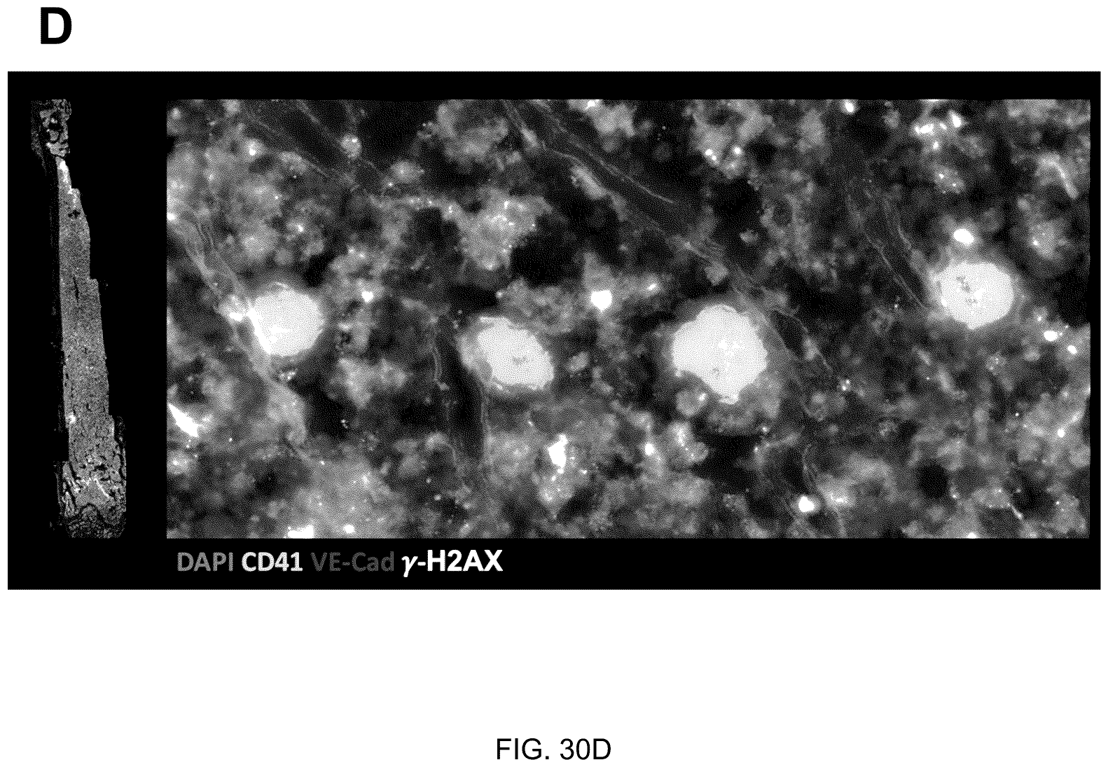

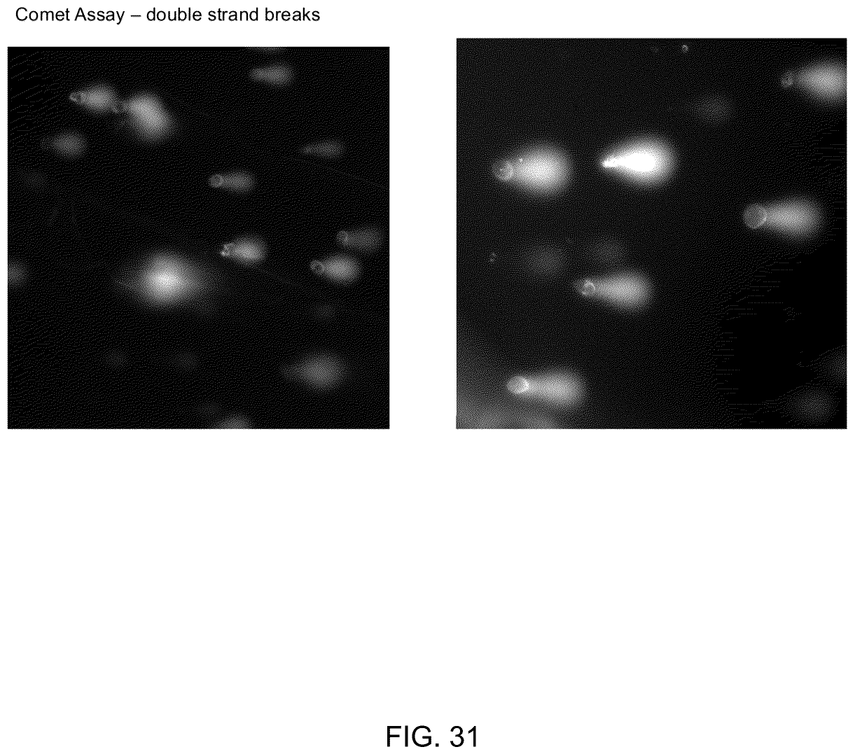

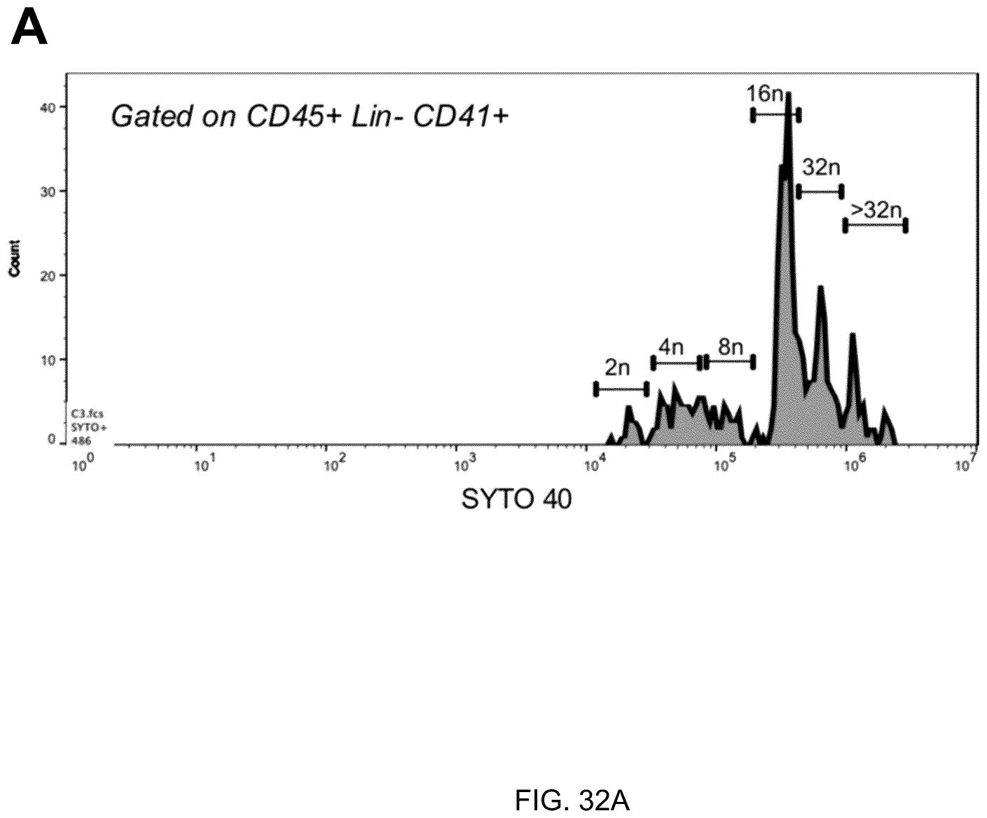

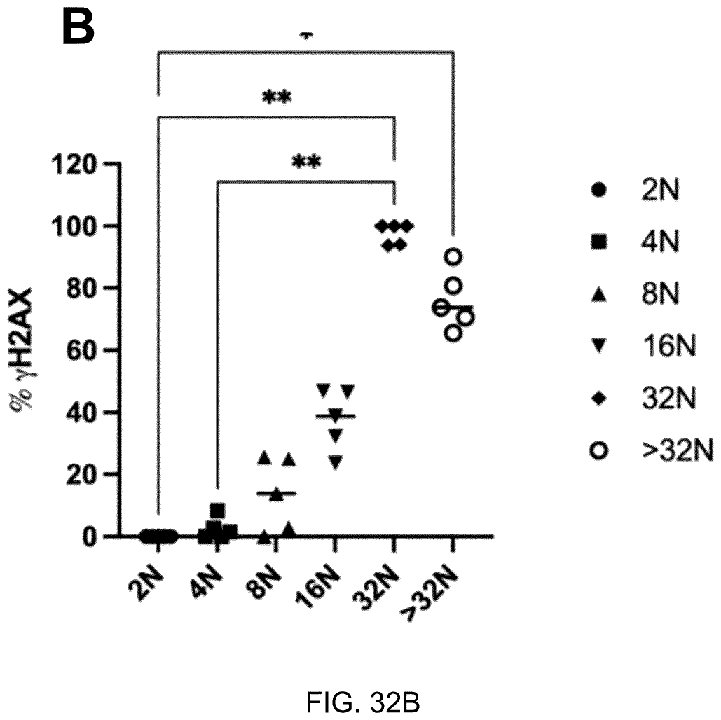

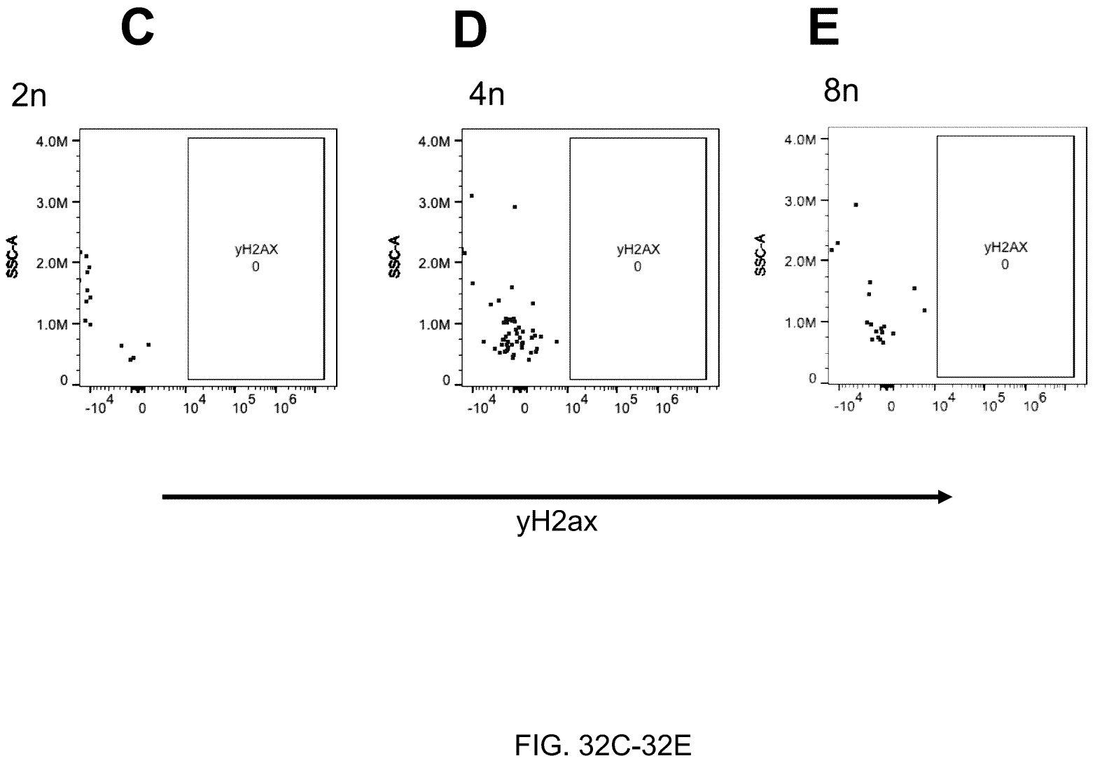

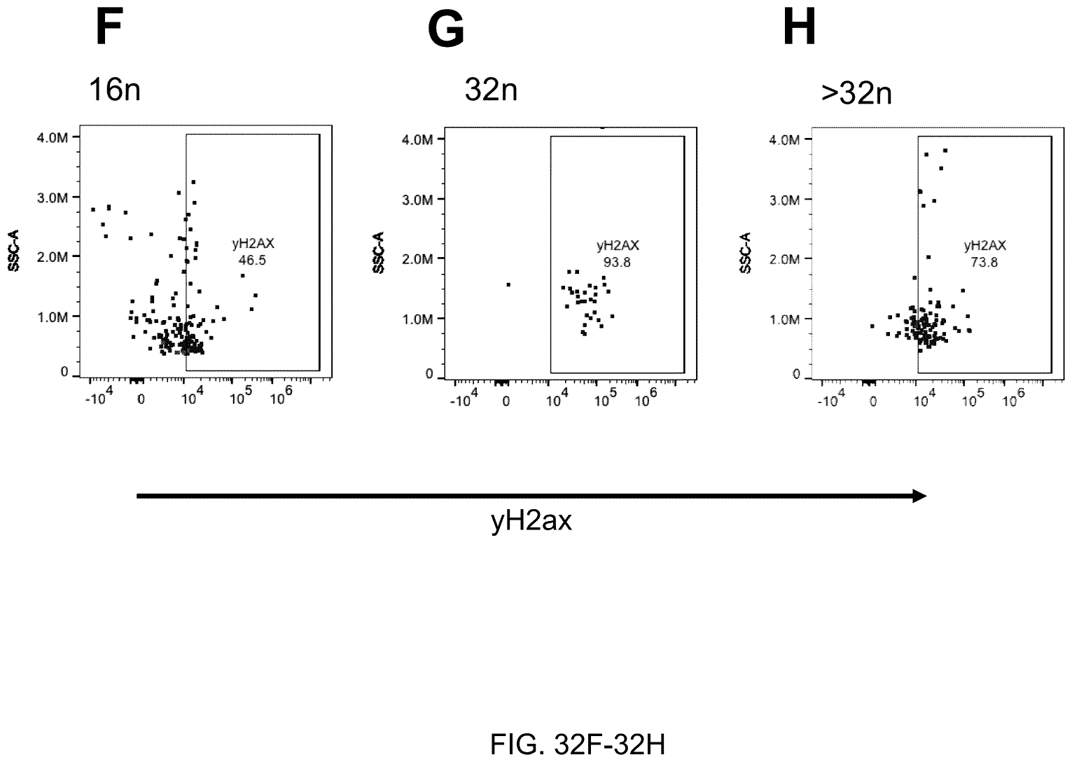

Gating on polidy status reveals a shift from light gray (2N) to dark gray (8BN) with an increasing level of γH2AX, indicating that Human BM MKs accumulate DNA damage (FIG. 29).

Key findings: Accumulation of DNA damage increases both MK and platelet counts. MKs accumulate DNA damage under normal physiology-scales with ploidy and is not toxic, as MKs then continue endomitosis and make platelets.

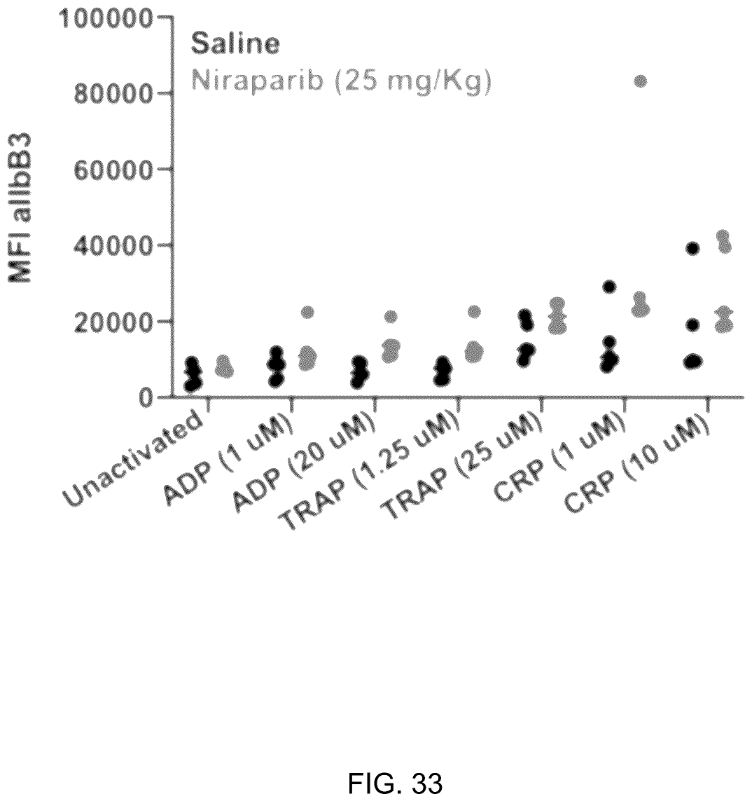

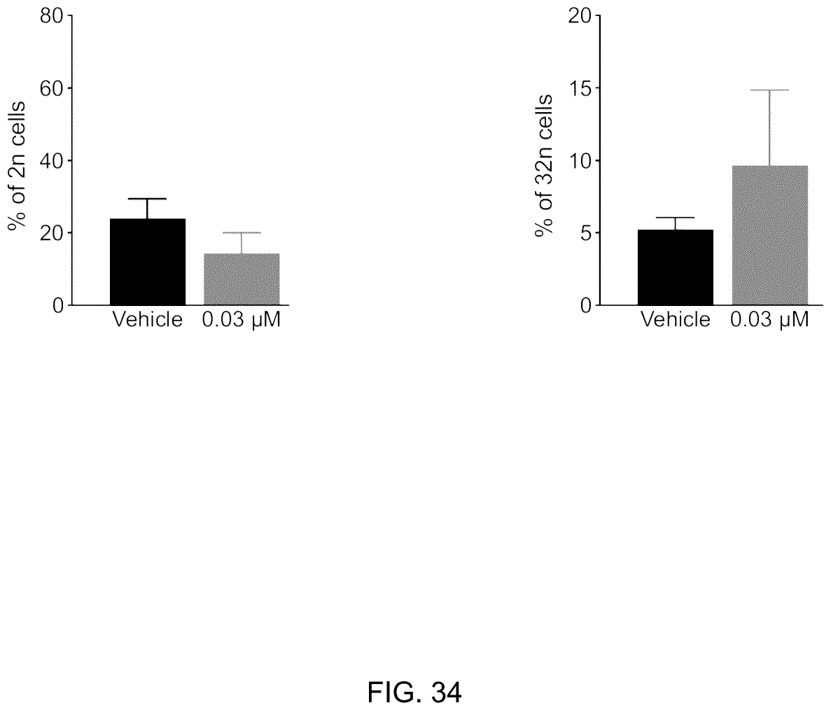

Niraparib treatment of mice does not affect platelet function (FIG. 33). Platelets were drawn from mice at sacrifice on D11, stimulated with agonists as indicated, and the MFI of activated αIIbβ3 measured by flow cytometry.

What is claimed herein is:

1. A method of treating a subject with thrombocytopenia, the method comprising administering to the subject at least one DNA damage reagent and/or at least one DNA damage repair inhibitor.

2. The method of claim 1, comprising administering to the subject a low dose of a DNA damage reagent or a DNA damage repair inhibitor.

3. The method of claim 1, wherein the DNA damage repair inhibitor is a poly-(ADP-ribose) polymerase (PARP) inhibitor.

4. The method of claim 1, wherein the PARP inhibitor is selected from the group consisting of:
niraparib; olaparib; rucaparib; talzaoparib; veliparib; pamiparib; CEP 9722; E7016;
iniparib; and 3-aminobenezamide.

5. The method of claim 1, wherein the PARP inhibitor is niraparib, olaparib, or rucaparib.

6. The method of claim 1, wherein the PARP inhibitor is niraparib or rucaparib.

7. The method of claim 1, wherein the PARP inhibitor is niraparib.

8. The method of claim 1, wherein the subject with thrombocytopenia has or is diagnosed as having a condition selected from the group consisting of:
immune thrombocytopenia purpura (ITP), aplastic anemia, human immunodeficiency virus (HIV), acquired immunodeficiency syndrome (AIDS), a myelodysplastic disorder, dehydration, vitamin B12 deficiency, folic acid deficiency, leukemia, myelodysplastic syndrome, liver failure, sepsis, viral infection, bacterial infection, leptospirosis, ACTN1-related thrombocytopenia, amegakaryocytic thrombocytopenia with radio-ulnar synostosis, ANKRD26 related thrombocytopenia, autosomal dominant thrombocytopenia, Bernard-Soulier syndrome, congenital amegakaryocytic thrombocytopenia, congenital amegakaryocytic thrombocytopenia and radioulnar synostosis, CYCS-related thrombocytopenia, Epstein syndrome, ETV6 related thrombocytopenia, Fanconi anemia, Filaminopathies A, FYB related thrombocytopenia, Glanzmann's thrombasthenia, GNE myopathy with congenital thrombocytopenia, Gray platelet syndrome, Harris platelet syndrome, Macrothrombocytopenia, May-Hegglin anomaly, MYH9-related disease, PRKACG-related thrombocytopenia, Paris-Trousseau thrombocytopenia/Jacobsen syndrome, Sebastian syndrome, SLFN14-related thrombocytopenia, Stormorken syndrome, TRPM7-related thrombocytopenia, thrombocytopenia absent radius syndrome, Tropomyosin 4-related thrombocytopenia, TUBB1-related thrombocytopenia, Upshaw-Schulman syndrome, Wiskott-Aldrich syndrome, X-linked thrombocytopenia, X-linked thrombocytopenia with thalassemia, thrombotic thrombocytopenia purpura, hemolytic-uremic syndrome, disseminated intravascular coagulation, paroxysmal nocturnal hemoglobinuria, antiphospholipid syndrome, systemic lupus erythematosus, post-transfusion purpura, neonatal alloimmune thrombocytopenia, hypersplenism, Dengue fever, Gaucher's disease, Zika virus, snakebite, Lyme disease, thrombocytapheresis, and Niemann-Pick disease.

9. The method of claim 1, wherein the subject with thrombocytopenia has been administered or is being administered a drug selected from the group consisting of:
valproic acid, methotrexate, carboplatin, interferon, isotretinoin, panobinostat, a H2 blocker, and a proton-pump inhibitor.

10. The method of claim 1, wherein the subject is undergoing or selected to undergo a surgical procedure or chemotherapy.

11. The method of claim 1, wherein the subject is pregnant.
